(19) United States
(12) Patent Application Publication
(10) Pub. No.: US 2013/0005837 A1
(43) Pub. Date: Jan. 3, 2013

(54) CANCER BIOMARKERS TO PREDICT RECURRENCE AND METASTATIC POTENTIAL

(75) Inventors: Carlos Moreno, Atlanta, GA (US); Qi Long, Atlanta, GA (US); Benjamin G. Barwick, Atlanta, GA (US)

(73) Assignee: Emory University, Atlanta, GA (US)

(21) Appl. No.: 13/519,731

(22) PCT Filed: Dec. 29, 2010
(86) PCT No.: PCT/US10/62293

§ 371 Date: Sep. 20, 2012

Related U.S. Application Data
(60) Provisional application No. 61/291,681, filed on Dec. 31, 2009.
(60) Provisional application No. 61/329,387, filed on Apr. 29, 2010.

(51) Int. Cl. C12Q 1/68; A61K 35/00; A61P 35/00; C40B 30/00

(52) U.S. Cl. 514/789; 435/6.14; 435/6.12; 435/6.11; 506/7

(57) ABSTRACT

Described herein are methods for predicting recurrence, progression, and metastatic potential of a prostate cancer in a subject. In certain embodiments, the methods comprise analyzing a sample from a subject for aberrant expression patters of one or more biomarkers disclosed herein. An increase or decrease in one or more biomarkers as compared to a standard indicates a recurrent, progressive, or metastatic prostate cancer.

This application claims priority to U.S. provisional application No. 61/291,681 filed Dec. 31, 2009 and U.S. provisional application No. 61/329,387 filed Apr. 29, 2010 both hereby incorporated by reference.

### BACKGROUND

Prostate cancer is the most commonly diagnosed noncutaneous neoplasm and second most common cause of cancer-related mortality in Western men. One of the important challenges in current prostate cancer research is to develop effective methods to determine whether a patient is likely to progress to the aggressive, metastatic disease in order to aid clinicians in deciding the appropriate course of treatment.

Various approaches using clinical parameters including prostate specific antigen (PSA) levels at time of initial diagnosis have been explored to predict disease progression. Although these models work well for men with extreme levels of PSA, the majority of men fall within an intermediate range characterized by a PSA level between 4-10 ng/ml and a Gleason score of 6 or 7. Current prognostic models of prostate cancer, including PSA, Gleason score and clinical stage fail to accurately predict disease progression, especially for men with intermediate disease. Thus there is a need for additional tests to complement and improve upon these existing approaches.

Technologies have been developed to exploit formalin-fixed paraffin-embedded (FFPE) tumor tissue samples for gene expression analysis. The DASL (cDNA-mediated Annealing, Selection, extension and Ligation) assay is a unique expression profiling platform based upon massively multiplexed RT-PCR applied in a microarray format allowing for the determination of expression of RNA isolated from FFPE tumor tissue samples in a high throughput format. See Bibikova et al., Am J Pathol 2004, 165:1799-1807 and Fan et al. Genome Res 2004, 14:878-885. The DASL assay has been used to identify a 16-gene set that correlates with prostate cancer relapse. Bibikova et al., Genomics 2007, 89:666-672. However, diagnosis of the progression for prostate cancer using molecular biomarkers is challenging because molecular expression may be limited by sampling at time of initial diagnosis, may not be present at time of initial diagnosis, or may occur as the disease progresses. See Sboner et al., BMC Med Genomics 2010, 3:8 and Nakagawa et al., PLoS ONE 2008, 3:e2318.

### SUMMARY

Provided are methods of predicting the recurrence, progression, and metastatic potential of a cancer in a subject, typically prostate cancer. The methods comprise selecting a subject at risk of recurrence, progression, or metastasis of cancer, detecting in a sample from the subject one or more biomarkers selected from the group consisting of CSPG2, WNT10B, E2F3, CDKN2A, TYMS, TGFB3, ALOX12, CD44, LAF4, CTNNA1, XPO1, PTGDS, SOX9, RELA, EPB49, SIM2, EDNRA, RAD23B, FBP1, TNFRSF1A, CCNG2, LETMD1, NOTCH3, ETV1, BID, SIM2, ANXA1, BCL2, miR-519d, miR-647, FOXO1A, SOX9, CLNS1A, PTGDS, XPO1, LETMD1, RAD23B, ABCC3, APC, CHEST, EDNRA, FRZB, HSPG2, TMPRSS2_ETV1 FUSION, CSPG2, WNT10B, E2F3, CDKN2A, TYMS, miR-103, miR-339, miR-183, miR-182, miR-136, and/or miR-221 to create a biomarker profile, analyzing the biomarker, and correlating an aberrant expression pattern to a heightened potential for recurrence, progression or metastasis of cancer.

In another embodiment, the biomarkers are selected from one or more of CTNNA1, XPO1, PTGDS, SOX9, RELA, EPB49, SIM2, and EDNRA. In certain embodiments, the panel includes at least two of the biomarkers, and typically includes at least three or at least four, or at least five, or at least six, or at least seven or at least eight biomarkers and includes at least one biomarker selected from CTNNA1, XPO1, PTGDS, SOX9, RELA, EPB49, SIM2, and EDNRA. In another embodiment, the biomarkers are selected from one or more, or two or more, or three or more, or four or more, or five or more, or six or more, or seven or more, or eight or more of RAD23B, FBP1, TNFRSF1A, CCNG2, LETMD1, NOTCH3, ETV1, BID, SIM2, and ANXA1, and miR-519d, and/or miR-647. Typically, one analyzes a sample from a subject for the presence of mRNA of one or more protein-coding genes RAD23B, FBP1, TNFRSF1A, CCNG2, LETMD1, NOTCH3, ETV1, BID, SIM2, ANXA1 and one or both microRNA of miR-519d and/or miR-647.

In certain embodiments, the panel includes at least two of the biomarkers, and typically includes at least three or at least four, or at least five, or at least six, or at least seven, or at least eight, or at least nine biomarkers and includes at least one biomarker selected from RAD23B, FBP1, TNFRSF1A, CCNG2, LETMD1, NOTCH3, ETV1, BID, SIM2, and ANXA1, and miR-519d, and/or miR-647.

An increase or decrease in one or more of the biomarkers as compared to a standard indicates a prostate cancer that is prone to recur, progress, and/or metastasize. Optionally, the methods further comprise detecting one or more biomarkers selected from the group consisting of FOXO1A, SOX9, CLNS1A, PTGDS, XPO1, LETMD1, RAD23B, ABCC3, APC, CHES1, EDNRA, FRZB, HSPG2, TMPRSS2_ETV1 FUSION, miR-103, miR-339, miR-183, miR-182, miR-136, and miR-221.

Also provided are methods of treating a subject diagnosed with prostate cancer comprising modifying the treatment regimen of the subject based on the results of the method of predicting the recurrence, progression, and/or metastatic potential of a prostate cancer in a subject. The treatment regimen is modified to be aggressive based on an increase in one or more biomarkers selected from the group consisting of CSPG2, WNT10B, E2F3, CDKN2A, and TYMS as compared to a standard, and a decrease in one or more biomarkers selected from the group consisting of TGFB3, ALOX12, CD44 and LAF4 as compared to a standard. The treatment regimen is further modified to be aggressive based on an increase in one or more biomarkers selected from the group consisting of CLNS1A, XPO1, LETMD1, RAD23B, TMPRSS2_ETV1 FUSION, ABCC3, SPC, CHES1, FRZB, HSPG2, miR-103, miR-339, miR-183, and miR-182 as compared to a standard, and a decrease in one or more biomarkers selected from the group consisting of FOXO1A, SOX9, PTGDS, EDNRA, miR-136, and miR-221 as compared to a standard. The treatment regimen is further modified to be aggressive based on an increased expression of RAD23B, FBP1, CCNG2, LETMD1, NOTCH3, ETV1, BID, SIM2, miR-519d and the decreased expression of TNFRSF1A, miR-647, and ANXA1.

Also provided are kits comprising one or more primers to detect expression of biomarkers selected from the group consisting of CSPG2, WNT10B, E2F3, CDKN2A, TYMS, TGFB3, ALOX12, CD44, and LAF4. The kits can further comprise one or more primers to detect expression of biomarkers selected from the group consisting of FOXO1A, SOX9, CLNS1A, PTGDS, XPO1, LETMD1, RAD23B, ABCC3, APC, CHES1, EDNRA, FRZB, HSPG2, TMPRSS2_ETV1 FUSION, miR-103, miR-339, miR-183, miR-182, miR-136, and miR-221. The kits can further comprise one or more primers to detect expression of biomarkers selected from the group consisting of RAD23B, FBP1, TNFRSF1A, CCNG2, LETMD1, NOTCH3, ETV1, BID, SIM2, ANXA1, miR-519d, and miR-647.

In certain embodiments, the disclosure relates to methods of predicting the recurrence, progression, and metastatic potential of a prostate cancer in a subject, the method comprising analyzing a sample from the subject for an aberrant expression pattern of four, five, six, seven, eight, nine or more biomarkers wherein at least one of the biomarkers is a microRNA. In certain embodiments, the mircoRNA is miR-519d, miR-647, miR-103, miR-339, miR-183, miR-182, miR-136, and/or miR-221.

In some embodiments, the panel includes at least two of the biomarkers, and typically includes at least three or at least four, or at least five, or at least six, or at least seven or at least eight biomarkers and correlates expression levels to the recurrence, progression, and potential of prostate cancer. Typically, one analyzes a sample from a subject for the presence of mRNA of one or more protein-coding genes and one or more miRNA. Typically, the subject previously had a partial or total prostate removal by surgery including portions of the prostate that contained cancerous cells.

In certain embodiments, the disclosure relates to analyzing biomarkers disclosed herein and correlating aberrant expression patterns to a likelihood of prostate cancer recurrence. Typically, analyzing comprises detecting mRNA or detecting protein levels directly such as, but not limited to, moving the samples through a separation medium and exposing fractions to antibodies with epitopes to certain sequences on the proteins, or identifying the biomarker using mass spectroscopy. Typically the mRNA or microRNA (miRNA) may be detected by amplification using primers and hybridization to a suitably labeled complimentary nucleic acid probe. Typically, the label is a fluorescent dye conjugated to the nucleic acid probe.

### DESCRIPTION OF DRAWINGS

FIG. 1 shows data on the time to recurrence survival analysis of Prostate cancer patients. (A) Kaplan-Meier analysis of the training set of 61 patients with complete clinical data that were separated based on the expression of RAD23B, FBP1, TNFRSF1A, LETMD1, NOTCH3, ETV1, BID, SIM2, ANXA1, and BCL2. (B) Kaplan-Meier analysis on the 35 validation cases with complete clinical data using this mRNA panel. (C) Kaplan-Meier analysis of the training set using the combined mRNA and miRNA panel of RAD23B, FBP1, TNFRSF1A, CCNG2, hsa-miR-647, LETMD1, NOTCH3, ETV1, hsa-miR-519d, BID, SIM2, and ANXA1. (D) Kaplan-Meier analysis of the validation set using the combined mRNA and miRNA panel.

FIGS. 2A-2C show characteristics of prostate cancer patients with and without TMPRSS2-ERG fusion. FIG. 2A shows a graph demonstrating that patients with TMPRSS2-ERG fusion positive tumors experienced a higher rate of biochemical recurrence opposed to those that did not have the gene fusion (log rank p-value=3.54×10⁻⁸). FIG. 2B shows a graph demonstrating that ERG expression was upregulated in TMPRSS2-ERG fusion positive tumors by 3.07-fold (p=3.48×10⁻¹¹, Student's t-test). FIG. 2C shows a graph confirming the microarray results presented in FIG. 2B with an RT-PCR assay. The RT-PCR assay confirmed increased ERG expression in TMPRSS2-ERG fusion positive tumors (p=8.13×10-10, Student's two-sided t-test).

FIG. 3 shows validated genes differentially expressed in TMPRSS2-ERG fusion positive tumors. Significance testing of genes differentially regulated in TMPRSS2-ERG fusion positive prostate tumors in the Toronto cohort of a 139 patients characterized on 502 genes (solid black line) was validated in a Swedish cohort of 455 patients characterized for 6,144 genes (dashed black line). Nine genes upregulated with TMPRSS2-ERG fusion in both cohorts are shown on top, while six genes downregulated in both cohorts are shown on the bottom

FIGS. 4A-4D show permutation testing of genes associated with TMPRSS2-ERG fusion. To determine significant differentially regulated genes associated with TMPRSS2-ERG fusion, 1,000 permutations of random class assignment estimated genes were performed with a false discovery rate (FDR) less than 5%. FIGS. 4A and 4B show Q-q plots of the Toronto cohort of 139 patients (FIG. 4A) and of the Swedish cohorts of 455 patients (FIG. 4B). In both cases ERG was distinctly the most overrepresented gene in TMPRSS2-ERG fusion positive tumors as depicted by box plots of ERG expression intensities for the Toronto (FIG. 4C) and Swedish cohorts (FIG. 4D).

FIG. 5 shows common genes prognostic of biochemical recurrence. Univariate Cox proportional hazards regression determined genes associated with biochemical recurrence in the Toronto cohort of 139 patients and a Minnesota cohort of 596 patients. Seven genes were identified in common; five genes were associated with recurrence, and two genes were associated with non-recurrence.

FIGS. 6A and 6B show Kaplan-Meier survival analysis of the Toronto cohort. FIG. 6A shows a Kaplan-Meier plot demonstrating the seven-gene expression recurrence score used to segregate patients into good and poor prognostic categories. (p=0.000167) FIG. 6B shows a Kaplan-Meier plot demonstrating that a mixed clinical model composed of Gleason score, TMPRSS2-ERG fusion status, and the seven-gene expression recurrence score is better able to prognosticate recurrence (p=4.15×10⁻⁷).

FIG. 7 shows data using Kaplan-Meier survival analysis. (A) Kaplan-Meier analysis of the training set of 42 Gleason 7 cases with complete clinical data using the mRNA panel of RAD23B, FBP1, TNFRSF1A, LETMD1, NOTCH3, ETV1, BID, SIM2, ANXA1, and BCL2. (B) Kaplan-Meier analysis of the 19 Gleason 7 cases in the validation set using the mRNA panel. (C) Kaplan-Meier analysis of the Gleason 7 cases in the training set using the combined mRNA and miRNA panel or RAD23B, FBP1, TNFRSF1A, CCNG2, hsa-miR-647, LETMD1, NOTCH3, ETV1, hsa-miR-519d, BID, SIM2, and ANXA1. (D) Kaplan-Meier analysis of the Gleason 7 cases in the validation set using the combined mRNA and miRNA panel.

### DETAILED DESCRIPTION

Described herein are methods for predicting the recurrence, progression, and/or metastatic potential of a cancer in a subject. The methods comprise selecting a subject at risk of recurrence, progression, or metastasis of prostate cancer, and detecting in a sample from a subject one or more biomarkers selected from the group consisting of CSPG2, WNT10B, E2F3, CDKN2A, TYMS, TGFB3, ALOX12, CD44, LAF4, CTNNA1, XPO1, PTGDS, SOX9, RELA, EPB49, SIM2, EDNRA, RAD23B, FBP1, TNFRSF1A, CCNG2, LETMD1, NOTCH3, ETV1, BID, SIM2, ANXA1, miR-519d, miR-647, FOXO1A, SOX9, CLNS1A, PTGDS, XPO1, LETMD1, RAD23B, ABCC3, APC, CHES1, EDNRA, FRZB, HSPG2, TMPRSS2_ETV1 FUSION, CSPG2, WNT10B, E2F3, CDKN2A, TYMS, miR-103, miR-339, miR-183, miR-182, miR-136, and miR-221 to create a biomarker profile. It is understood that detection of biomarker may be by detection of the gene, mRNA, translated protein, microRNA or other indicator that suggests gene expression.

In certain embodiments, one analyzes a sample from the subject for aberrant expression of RAD23B, FBP1, TNFRSF1A, CCNG2, LETMD1, NOTCH3, ETV1, BID, SIM2, ANXA1, miR-519d, miR-647, and correlating such expression to a likelihood of recurrence, progression, or metastasis of prostate cancer. In certain embodiments, the aberrant expression is increased expression of RAD23B, FBP1, CCNG2, LETMD1, NOTCH3, ETV1, BID, SIM2, miR-519d and the decreased expression of TNFRSF1A, miR-647, and ANXA1.

An increase or decrease in one or more of the biomarkers as compared to a standard indicates a prostate cancer that is prone to recur, progress, and/or metastasize. Optionally, the sample comprises prostate tumor tissue. Optionally, the prostate cancer comprises a TMPRSS2-ERG fusion-positive prostate cancer.

Optionally, the detected biomarkers comprise two or more, three or more, four or more, five or more, six or more, seven or more, eight or more biomarkers selected from the group consisting of RAD23B, FBP1, TNFRSF1A, CCNG2, LETMD1, NOTCH3, ETV1, BID, SIM2, ANXA1, combination with miR-519d and/or miR-647. For example, the detected biomarkers can comprise detecting miR-519 and/or miR-647 in combination with RAD23, FBP1, CCNG2, LETMD1, NOTCH3, ETV1, BID, SIM2, and ANXA1; or RAD23, FBP1, TNFRSF1A, LETMD1, NOTCH3, ETV1, BID, SIM2, and ANXA1; or RAD23, FBP1, TNFRSF1A, CCNG2, NOTCH3, ETV1, BID, SIM2, and ANXA1; or RAD23, FBP1, TNFRSF1A, CCNG2, LETMD1, ETV1, BID, SIM2, and ANXA1; or RAD23, FBP1, TNFRSF1A, CCNG2, LETMD1, NOTCH3, BID, SIM2, and ANXA1; or RAD23, FBP1, TNFRSF1A, CCNG2, LETMD1, NOTCH3, ETV1, SIM2, and ANXA1; or RAD23, FBP1, TNFRSF1A, CCNG2, LETMD1, NOTCH3, ETV1, BID, and ANXA1; or RAD23, FBP1, TNFRSF1A, CCNG2, LETMD1, NOTCH3, ETV1, BID, and SIM2; or TNFRSF1A, CCNG2, LETMD1, NOTCH3, ETV1, BID, SIM2, and ANXA1; or RAD23, CCNG2, LETMD1, NOTCH3, ETV1, BID, SIM2, and ANXA1; or RAD23, FBP1, LETMD1, NOTCH3, ETV1, BID, SIM2, and ANXA1; or RAD23, FBP1, TNFRSF1A, NOTCH3, ETV1, BID, SIM2, and ANXA1; or RAD23, FBP1, TNFRSF1A, CCNG2, ETV1, BID, SIM2, and ANXA; or RAD23, FBP1, TNFRSF1A, CCNG2, LETMD1, BID, SIM2, and ANXA1; or RAD23, FBP1, TNFRSF1A, CCNG2, LETMD1, NOTCH3, SIM2, and ANXA1; or RAD23, FBP1, TNFRSF1A, CCNG2, LETMD1, NOTCH3, ETV1, and ANXA1; or RAD23, FBP1, TNFRSF1A, CCNG2, LETMD1, NOTCH3, ETV1, and BID; or FBP1, CCNG2, LETMD1, NOTCH3, ETV1, BID, SIM2, and ANXA1; or FBP1, TNFRSF1A, LETMD1, NOTCH3, ETV1, BID, SIM2, and ANXA1; or FBP1, TNFRSF1A, CCNG2, NOTCH3, ETV1, BID, SIM2, and ANXA1; or FBP1, TNFRSF1A, CCNG2, LETMD1, ETV1, BID, SIM2, and ANXA1; or FBP1, TNFRSF1A, CCNG2, LETMD1, NOTCH3, BID, SIM2, and ANXA1; or FBP1, TNFRSF1A, CCNG2, LETMD1, NOTCH3, ETV1, SIM2, ANXA1; or FBP1, TNFRSF1A, CCNG2, LETMD1, NOTCH3, ETV1, BID, and ANXA1; or FBP1, TNFRSF1A, CCNG2, LETMD1, NOTCH3, ETV1, BID, and SIM2; or RAD23, TNFRSF1A, LETMD1, NOTCH3, ETV1, BID, SIM2, and ANXA1; or RAD23TNFRSF1A, CCNG2, NOTCH3, ETV1, BID, SIM2, and ANXA1; or RAD23, TNFRSF1A, CCNG2, LETMD1, ETV1, BID, SIM2, and ANXA1; or RAD23, TNFRSF1A, CCNG2, LETMD1, NOTCH3, BID, SIM2, and ANXA1; or RAD23, TNFRSF1A, CCNG2, LETMD1, NOTCH3, ETV1, SIM2, and ANXA1; or RAD23, TNFRSF1A, CCNG2, LETMD1, NOTCH3, ETV1, BID, and ANXA1; or RAD23, TNFRSF1A, CCNG2, LETMD1, NOTCH3, ETV1, BID, and SIM2; or RAD23, FBP1, CCNG2, NOTCH3, ETV1, BID, SIM2, and ANXA1; or RAD23, FBP1, CCNG2, LETMD1, ETV1, BID, SIM2, and ANXA1; or RAD23, FBP1, CCNG2, LETMD1, NOTCH3, BID, SIM2, and ANXA1; or RAD23, FBP1, CCNG2, LETMD1, NOTCH3, ETV1, SIM2, and ANXA1; or RAD23, FBP1, CCNG2, LETMD1, NOTCH3, ETV1, BID, and SIM2; or RAD23, FBP1, TNFRSF1A, LETMD1, ETV1, BID, SIM2, and ANXA1; or RAD23, FBP1, TNFRSF1A, LETMD1, NOTCH3, BID, SIM2, and ANXA1; or RAD23, FBP1, TNFRSF1A, LETMD1, NOTCH3, ETV1, SIM2, and ANXA1; or RAD23, FBP1, TNFRSF1A, LETMD1, NOTCH3, ETV1, BID, and ANXA1; or RAD23, FBP1, TNFRSF1A, LETMD1, NOTCH3, ETV1, BID, and SIM2; or RAD23, FBP1, TNFRSF1A, CCNG2, NOTCH3, BID, SIM2, and ANXA1; or RAD23, FBP1, TNFRSF1A, CCNG2, NOTCH3, ETV1, SIM2, and ANXA1; or RAD23, FBP1, TNFRSF1A, CCNG2, NOTCH3, ETV1, BID, and ANXA1; or RAD23, FBP1, TNFRSF1A, CCNG2, NOTCH3, ETV1, BID, and SIM2; or RAD23, FBP1, TNFRSF1A, CCNG2, LETMD1, ETV1, SIM2, and ANXA1; or RAD23, FBP1, TNFRSF1A, CCNG2, LETMD1, ETV1, BID, and ANXA1; or RAD23, FBP1, TNFRSF1A, CCNG2, LETMD1, ETV1, BID, and SIM2; or RAD23, FBP1, TNFRSF1A, CCNG2, LETMD1, NOTCH3, BID, and ANXA1; or RAD23, FBP1, TNFRSF1A, CCNG2, LETMD1, NOTCH3, BID, and SIM2; or CCNG2, LETMD1, NOTCH3, ETV1, BID, SIM2, and ANXA1; or RAD23, LETMD1, NOTCH3, ETV1, BID, SIM2, and ANXA1; or RAD23, FBP1, NOTCH3, ETV1, BID, SIM2, and ANXA1; or RAD23, FBP1, TNFRSF1A, ETV1, BID, SIM2, and ANXA1; or RAD23, FBP1, TNFRSF1A, CCNG2, BID, SIM2, and ANXA1; or RAD23, FBP1, TNFRSF1A, CCNG2, LETMD1, SIM2, and ANXA1; or RAD23, FBP1, TNFRSF1A, CCNG2, LETMD1, NOTCH3, and ANXA1; or RAD23, FBP1, TNFRSF1A, CCNG2, LETMD1, NOTCH3, and ANXA1; or RAD23, FBP1, TNFRSF1A, CCNG2, LETMD1, NOTCH3, and ETV1; or FBP1, LETMD1, NOTCH3, ETV1, BID, SIM2, and ANXA1; or FBP1, TNFRSF1A, NOTCH3, ETV1, BID, SIM2, and ANXA1; or FBP1, TNFRSF1A, CCNG2, LETMD1, BID, SIM2, and ANXA1; or FBP1, TNFRSF1A, CCNG2, LETMD1, NOTCH3, SIM2, and ANXA1; or FBP1, TNFRSF1A, CCNG2, LETMD1, NOTCH3, ETV1, and ANXA1; or FBP1, TNFRSF1A, CCNG2, LETMD1, NOTCH3, ETV1, and BID; or RAD23, TNFRSF1A, NOTCH3, ETV1, BID, SIM2, and ANXA1; or RAD23, TNFRSF1A, CCNG2, ETV1, BID, SIM2, and ANXA1; or RAD23, TNFRSF1A, CCNG2, LETMD1, NOTCH3, SIM2, and ANXA1; or RAD23, TNFRSF1A, CCNG2, LETMD1, NOTCH3, ETV1, and ANXA1; or RAD23, TNFRSF1A, CCNG2, LETMD1, NOTCH3, ETV1, and BID; or RAD23, FBP1, CCNG2, LETMD1, NOTCH3, SIM2, and ANXA1; or RAD23, FBP1, CCNG2, ETV1, BID, SIM2, and ANXA1; or RAD23, FBP1, CCNG2, LETMD1, NOTCH3, ETV1, and ANXA1; or RAD23, FBP1, CCNG2, LETMD1, NOTCH3, ETV1, and BID; or TNFRSF1A, LETMD1, NOTCH3, ETV1, BID, SIM2, and ANXA1; or RAD23, FBP1, TNFRSF1A, LETMD1, NOTCH3, SIM2, and ANXA1; or RAD23, FBP1, TNFRSF1A, LETMD1, NOTCH3, ETV1, and ANXA1; or RAD23, FBP1, TNFRSF1A, LETMD1, NOTCH3, ETV1, and BID; or TNFRSF1A, CCNG2, NOTCH3, ETV1, BID, SIM2, and ANXA1; or RAD23, CCNG2, NOTCH3, ETV1, BID, SIM2, and ANXA1; or RAD23, FBP1, TNFRSF1A, CCNG2, NOTCH3, SIM2, and ANXA1; or RAD23, FBP1, TNFRSF1A, CCNG2, NOTCH3, ETV1, and ANXA1; or RAD23, FBP1, TNFRSF1A, CCNG2, NOTCH3, ETV1, and BID; or TNFRSF1A, CCNG2, LETMD1, ETV1, BID, SIM2, and ANXA1; or RAD23, CCNG2, LETMD1, ETV1, BID, SIM2, and ANXA1; or RAD23, FBP1, TNFRSF1A, CCNG2, LETMD1, ETV1, and ANXA1; or RAD23, FBP1, TNFRSF1A, CCNG2, LETMD1, ETV1, and BID; or TNFRSF1A, CCNG2, LETMD1, NOTCH3, BID, SIM2, and ANXA1; or RAD23, CCNG2, LETMD1, NOTCH3, BID, SIM2, and ANXA1; or RAD23, FBP1, LETMD1, NOTCH3, BID, SIM2, and ANXA1; or RAD23, FBP1, TNFRSF1A, NOTCH3, BID, SIM2, and ANXA1; or RAD23, FBP1, TNFRSF1A, CCNG2, LETMD1, NOTCH3, and BID; or TNFRSF1A, CCNG2, LETMD1, NOTCH3, ETV1, SIM2, and ANXA1; or RAD23, CCNG2, LETMD1, NOTCH3, ETV1, SIM2, and ANXA1; or RAD23, FBP1, LETMD1, NOTCH3, ETV1, SIM2, and ANXA1; or RAD23, FBP1, TNFRSF1A, NOTCH3, ETV1, SIM2, and ANXA1; or RAD23, FBP1, TNFRSF1A, CCNG2, ETV1, SIM2, and ANXA1; or RAD23, FBP1, TNFRSF1A, CCNG2, LETMD1, ETV1, and SIM2; or TNFRSF1A, CCNG2, LETMD1, NOTCH3, ETV1, BID, and ANXA1; or RAD23, CCNG2, LETMD1, NOTCH3, ETV1, BID, and ANXA1; or RAD23, FBP1, LETMD1, NOTCH3, ETV1, BID, and ANXA1; or RAD23, FBP1, TNFRSF1A, NOTCH3, ETV1, BID, and ANXA1; or RAD23, FBP1, TNFRSF1A, CCNG2, LETMD1, NOTCH3, ETV1, BID, and ANXA1; or RAD23, FBP1, TNFRSF1A, CCNG2, ETV1, BID, SIM2, and ANXA1; or RAD23, FBP1, TNFRSF1A, CCNG2, LETMD1, BID, and ANXA1; or RAD23, FBP1, TNFRSF1A, CCNG2, LETMD1, NOTCH3, ETV1, and BID; or TNFRSF1A, CCNG2, LETMD1, NOTCH3, ETV1, BID, and SIM2; or RAD23, CCNG2, LETMD1, NOTCH3, ETV1, BID, and SIM2; or RAD23, FBP1, LETMD1, NOTCH3, ETV1, BID, and SIM2; or RAD23, FBP1, TNFRSF1A, NOTCH3, ETV1, BID, and SIM2; or RAD23, FBP1, TNFRSF1A, CCNG2, ETV1, BID, SIM2; or RAD23, FBP1, TNFRSF1A, CCNG2, LETMD1, BID, and SIM2.

Optionally, multiple biomarkers are detected. Detection can comprise identifying an RNA expression pattern. An increase in one or more of the biomarkers selected from the group consisting of CSPG2, WNT10B, E2F3, CDKN2A, and TYMS as compared to a standard indicates a prostate cancer that is prone to recur, progress, and/or metastasize, whereas a decrease indicates a prostate cancer that is unlikely to recur and is slow to progress and/or metastasize. A decrease in one or more of the biomarkers selected from the group consisting of TGFB3, ALOX12, CD44, and LAF4 as compared to a standard indicates a prostate cancer that is prone to recur, progress, and/or metastasize, whereas an increase indicates a prostate cancer that is unlikely to recur and is slow to progress and/or metastasize. Optionally, the detected biomarkers comprise two or more, three or more, four or more, five or more, six or more, seven or more, eight or more, or all nine biomarkers selected from the group consisting of CSPG2, WNT10B, E2F3, CDKN2A, TYMS, TGFB3, ALOX12, CD44, and LAF4. For example, the detected biomarkers can comprise CSPG2 and E2F3. For example, the detected biomarkers can comprise CDKN2A, TGFB3, and LAF4. For example, the detected biomarkers can comprise WNT10B, E2F3, ALOX12, and CD44. For example, the detected biomarkers can comprise CSPG2, CDKN2A, TYMS, TGFB3, and LAF4. For example, the detected biomarkers can comprise CSPG2, WNT10B, E2F3, TYMS, ALOX12, and CD44. For example, the detected biomarkers can comprise CSPG2, WNT10B, E2F3, CDKN2A, TYMS, CD44, and LAF4. For example, the detected biomarkers can comprise WNT10B, E2F3, CDKN2A, TYMS, TGFB3, ALOX12, CD44, and LAF4. Optionally, the detected biomarkers comprise biomarkers from the group consisting of CSPG2, WNT10B, E2F3, CDKN2A, TYMS, TGFB3, ALOX12, CD44, and LAF4.

Optionally, the methods further comprise detecting in a sample from the subject one or more biomarkers selected from the group consisting of FOXO1A, SOX9, CLNS1A, PTGDS, XPO1, LETMD1, RAD23B, ABCC3, APC, CHES1, EDNRA, FRZB, HSPG2, TMPRSS2_ETV1 FUSION, miR-103, miR-339, miR-183, miR-182, miR-136, and miR-221.

Optionally, multiple biomarkers are detected. Detection can comprise identifying an RNA expression pattern. An increase in one or more biomarkers selected from the group consisting of CLNS1A, XPO1, LETMD1, RAD23B, TMPRSS2_ETV1 FUSION, ABCC3, APC, CHES1, FRZB, HSPG2, miR-103, miR-339, miR-183, and miR-182 as compared to a control indicates a prostate cancer that is prone to recur, progress, and/or metastasize, whereas a decrease indicates the opposite. A decrease in one or more biomarkers selected from the group consisting of FOXO1A, SOX9, EDNRA, PTGDS, miR-136, and miR-221 as compared to a standard indicates a prostate cancer that is prone to recur, progress, and/or metastasize, whereas an increase indicates the opposites. Optionally, the detected biomarkers comprise two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, or all twenty biomarkers selected from the group consisting of FOXO1A, SOX9, CLNS1A, PTGDS, XPO1, LETMD1, RAD23B, ABCC3, APC, CHES1, EDNRA, FRZB, HSPG2, TMPRSS2_ETV1 FUSION, miR-103, miR-339, miR-183, miR-182, miR-136, and miR-221. For example, the detected biomarkers can comprise FOXO1A and SOX9. For example, the detected biomarkers can comprise SOX9, CLNS1A, and miR-136. For example, the detected biomarkers can comprise FOXO1A, PTGDS, XPO1, and RAD23B. For example, the detected biomarkers can comprise CLNS1A, LETMD1, FRZB, miR-136, and miR-182. For example, the selected biomarkers can comprise FOXO1A, SOX9, CLNS1A, PTGDS, miR-339, and miR-183. For example, the selected biomarkers can comprise FOXO1A, CLNS1A, PTGDS, XPO1, FRZB, miR-182, and miR-183. For example, the selected biomarkers can comprise FOXO1A, CLNS1A, PTGDS, XPO1, LETMD1, miR-103, miR-339, and miR-183. For example, the selected biomarkers can comprise SOX9, CLNS1A, PTGDS, XPO1, LETMD1, RAD23B, TMPRSS2_ETV1 FUSION, miR-103, miR-339, and miR-182. For example, the selected biomarkers can comprise FOXO1A, SOX9, CLNS1A, XPO1, RAD23B, ABCC3, EDNRA, FRZB, TMPRSS2_ETV1 FUSION, and miR-339. For example, the selected biomarkers can comprise FOXO1A, SOX9, CLNS1A, PTGDS, XPO1, miR-339, miR-183, miR-182, miR-136, and miR-221. For example, the selected biomarkers can comprise FOXO1A, SOX9, CLNS1A, PTGDS, XPO1, LETMD1, RAD23B, ABCC3, APC, CHES1, and FRZB. For example, the selected biomarkers can comprise FOXO1A, SOX9, CLNS1A, PTGDS, XPO1, LETMD1, RAD23B, ABCC3, APC, EDNRA, HSPG2, and TMPRSS2_ETV1 FUSION. For example, the selected biomarkers can comprise FOXO1A, SOX9, CLNS1A, PTGDS, XPO1, LETMD1, RAD23B, ABCC3, APC, CHES1, EDRNA, FRZB, and HSPG2. For example, the selected biomarkers can comprise FOXO1A, SOX9, CLNS1A, PTGDS, XPO1, LETMD1, RAD23B, ABCC3, APC, CHES1, EDRNA, FRZB, HSPG2, and TMPRSS2_ETV1 FUSION. For example, the selected biomarkers can comprise FOXO1A, SOX9, CLNS1A, PTGDS, XPO1, LETMD1, RAD23B, ABCC3, APC, CHES1, EDRNA, FRZB, HSPG2, TMPRSS2_ETV1 FUSION, and miR-221. For example, the selected biomarkers can comprise FOXO1A, SOX9, CLNS1A, PTGDS, XPO1, LETMD1, RAD23B, ABCC3, APC, CHES1, EDRNA, FRZB, HSPG2, TMPRSS2_ETV1 FUSION, miR-103, and miR-339. For example, the selected biomarkers can comprise FOXO1A, SOX9, CLNS1A, PTGDS, XPO1, LETMD1, RAD23B, ABCC3, APC, CHES1, EDRNA, FRZB, HSPG2, TMPRSS2_ETV1 FUSION, miR-103, miR-339, and miR-183. For example, the selected biomarkers can comprise FOXO1A, SOX9, CLNS1A, PTGDS, XPO1, LETMD1, RAD23B, ABCC3, APC, CHES1, EDRNA, FRZB, HSPG2, TMPRSS2_ETV1 FUSION, miR-103, miR-339, miR-183, and miR-182. For example, the selected biomarkers can comprise FOXO1A, SOX9, CLNS1A, PTGDS, XPO1, LETMD1, RAD23B, ABCC3, APC, CHES1, EDRNA, FRZB, HSPG2, TMPRSS2_ETV1 FUSION, miR-339, miR-183, miR-182, miR-136, and miR-221. Optionally, the selected biomarkers comprise biomarkers selected from the group consisting of FOXO1A, SOX9, CLNS1A, PTGDS, XPO1, LETMD1, RAD23B, ABCC3, APC, CHES1, EDNRA, FRZB, HSPG2, TMPRSS2_ETV1 FUSION, miR-103, miR-339, miR-183, miR-182, miR-136, and miR-221.

Optionally, the detecting step comprises detecting mRNA levels of the biomarker. The mRNA detection can, for example, comprise reverse-transcription polymerase chain reaction (RT-PCR), quantitative real-time PCR (qRT-PCR), Northern analysis, microarray analysis, and cDNA-mediated annealing, selection, extension, and ligation (DASL) assay (Illumina, Inc.; San Diego, Calif.). Preferably, the RNA detection comprises the cDNA-mediated annealing, selection, extension, and ligation (DASL) assay (Illumina, Inc.). Optionally, the detecting step comprises detecting miRNA levels of the biomarker. The miRNA detection can, for example, comprise miRNA chip analysis, Northern analysis, RNase protection assay, in situ hybridization, miRNA expression profiling panels designed for the DASL assay (Illumina, Inc.), or a modified reverse transcription quantitative real-time polymerase chain reaction assay (qRT-PCR). Preferably the miRNA detection comprises the miRNA expression profiling panels designed for the DASL assay (Illumina, Inc.). Optionally, the detecting step comprises detecting mRNA and miRNA levels of the biomarker. The analytical techniques used to determine mRNA and miRNA expression are known. See, e.g., Sambrook et al., Molecular Cloning: A Laboratory Manual, 3^{rd} Ed., Cold Spring Harbor Press, Cold Spring Harbor, N.Y. (2001), Yin et al., Trends Biotechnol. 26:70-6 (2008); Wang and Cheng, Methods Mol. Biol. 414:183-90 (2008); Einat, Methods Mol. Biol. 342:139-57 (2006).

Comparing the mRNA or miRNA biomarker content with a biomarker standard includes comparing mRNA or miRNA content from the subject with the mRNA or miRNA content of a biomarker standard. Such comparisons can be comparisons of the presence, absence, relative abundance, or combination thereof of specific mRNA or miRNA molecules in the sample and the standard. Many of the analytical techniques discussed above can be used alone or in combination to provide information about the mRNA or miRNA content (including presence, absence, and/or relative abundance information) for comparison to a biomarker standard. For example, the DASL assay can be used to establish a mRNA or miRNA profile for a sample from a subject and the abundances of specific identified molecules can be compared to the abundances of the same molecules in the biomarker standard.

Optionally, the detecting step comprises detecting the protein expression levels of the protein-coding gene biomarkers. The protein-coding gene biomarkers can comprise CSPG2, WNT10B, E2F3, CDKN2A, TYMS, TGFB3, ALOX12, CD44, LAF4, FOXO1A, SOX9, CLNS1A, PTGDS, XPO1, LETMD1, RAD23B, ABCC3, APC, CHES1, EDNRA, FRZB, HSPG2, and TMPRSS2_ETV1 FUSION. The protein detection can, for example, comprise an assay selected from the group consisting of Western blot, enzyme-linked immunosorbent assay (ELISA), enzyme immunoassay (EIA), radioimmunoassay (RIA), immunohistochemistry, and protein array. The analytical techniques used to determine protein expression are known. See, e.g., Sambrook et al., Molecular Cloning: A Laboratory Manual, 3^{rd} Ed., Cold Spring Harbor Press, Cold Spring Harbor, N.Y. (2001).

Biomarker standards can be predetermined, determined concurrently, or determined after a sample is obtained from the subject. Biomarker standards for use with the methods described herein can, for example, include data from samples from subjects without prostate cancer, data from samples from subjects with prostate cancer that is not a progressive, recurrent, and/or metastatic prostate cancer, and data from samples from subjects with prostate cancer that is a progressive, recurrent, and/or metastatic prostate cancer. Comparisons can be made to multiple biomarker standards. The standards can be run in the same assay or can be known standards from a previous assay.

Also provided herein are methods of treating a subject with prostate cancer. The methods comprise modifying a treatment regimen of the subject based on the results of any of the methods of predicting the recurrence, progression, and metastatic potential of a prostate cancer in a subject. Optionally, the treatment regimen is modified to be aggressive based on an increase in one or more biomarkers selected from the group consisting of CSPG2, WNT10B, E2F3, CDKN2A, and TYMS as compared to a standard. Optionally, the treatment regimen is modified to be aggressive based on a decrease in one or more biomarkers selected from the group consisting of TGFB3, ALOX12, CD44, and LAF4 as compared to the standard. Optionally, the treatment regimen is modified to be aggressive based on a combination of an increase in one or more biomarkers selected from the group consisting of CSPG2, WNT10B, E2F3, CDKN2A, and TYMS as compared to a standard, and a decrease in one or more biomarkers selected from the group consisting of TGFB3, ALOX12, CD44, and LAF4 as compared to a standard. Optionally, the treatment regimen is further modified to be aggressive based on an increase in one or more biomarkers selected from the group consisting of CLNS1A, XPO1, LETMD1, RAD23B, TMPRSS2_ETV1 FUSION, ABCC3, APC, CHES1, FRZB, HSPG2, miR-103, miR-339, miR-183 and miR-182 as compared to a standard. Optionally, the treatment regimen is further modified to be aggressive based on a decrease in one or more biomarkers selected from the group consisting of FOXO1A, SOX9, PTGDS, EDNRA, miR-136, and miR-221 as compared to a standard. Optionally, the treatment regimen is further modified to be aggressive based on a combination of an increase in one or more biomarkers selected from the group consisting of CLNS1A, XPO1, LETMD1, RAD23B, TMPRSS2_ETV1 FUSION, ABCC3, APC, CHES1, FRZB, HSPG2, miR-103, miR-339, miR-183, and miR-182 and a decrease in one or more biomarkers selected from the group consisting of FOXO1A, SOX9, PTGDS, EDNRA, miR-136, and miR-221 as compared to a standard.

In certain embodiments, the treatment regimen is further modified to be aggressive based on an aberrant pattern of expression when analyzing miR-519d and/or miR-647 and four, five, six, seven, eight or more markers selected from the group consisting of RAD23B, FBP1, TNFRSF1A, CCNG2, LETMD1, NOTCH3, ETV1, BID, SIM2, and ANXA1.

Also provided are kits comprising primers to detect the expression of one or more biomarkers selected from the group consisting of CSPG2, WNT10B, E2F3, CDKN2A, TYMS, TGFB3, ALOX12, CD44, and LAF4. Optionally, the kits further comprise primers to detect the expression of one or more biomarkers selected from the group consisting of FOXO1A, SOX9, CLNS1A, PTGDS, XPO1, LETMD1, RAD23B, and TMPRSS2_ETV1, and primers to detect the expression of one or more biomarkers selected from the group consisting of miR-103, miR-339, miR-183, miR-182, miR-136, and miR-221. Optionally, directions to use the primers provided in the kit to predict the progression and metastatic potential of prostate cancer in a subject, materials needed to obtain RNA in a sample from a subject, containers for the primers, or reaction vessels are included in the kit.

Also provided are arrays consisting of probes to one or more of the biomarkers selected from the group consisting of CSPG2, WNT10B, E2F3, CDKN2A, TYMS, TGFB3, ALOX12, CD44, and LAF4. Optionally, the arrays further consist of probes to one or more biomarkers selected from the group consisting of FOXO1A, SOX9, CLNS1A, PTGDS, XPO1, LETMD1, RAD23B, ABCC3, APC, CHES1, EDNRA, FRZB, HSPG2, TMPRSS2_ETV1 FUSION, miR-103, miR-339, miR-183, miR-182, miR-136, and miR-221.

The arrays provided herein can be a DNA microarray, an RNA microarray, a miRNA microarray, or an antibody array. Arrays are known in the art. See, e.g., Dufva, Methods Mol. Biol. 529:1-22 (2009); Plomin and Schalk k, Dev. Sci. 10:1):19-23 (2007); Kopf and Zharhary, Int. J. Biochem. Cell Biol. 39(7-8):1305-17 (2007); Haab, Curr. Opin. Biotechnol. 17(4):415-21 (2006); Thomson et al., Nat. Methods 1:47-53 (2004).

As used herein, subject can be a vertebrate, more specifically a mammal (e.g., a human, horse, cat, dog, cow, pig, sheep, goat mouse, rabbit, rat, and guinea pig), birds, reptiles, amphibians, fish, and any other animal. The term does not denote a particular age. Thus, adult and newborn subjects are intended to be covered. As used herein, patient or subject may be used interchangeably and can refer to a subject afflicted with a disease or disorder (e.g., prostate cancer). The term patient or subject includes human and veterinary subjects.

As used herein a subject at risk for recurrence, progression, or metastasis of prostate cancer refers to a subject who currently has prostate cancer, a subject who previously has had prostate cancer, or a subject at risk of developing prostate cancer. A subject at risk of developing prostate cancer can be genetically predisposed to prostate cancer, e.g., a family history or have a mutation in a gene that causes prostate cancer. Alternatively a subject at risk of developing prostate cancer can show early signs or symptoms of prostate cancer, such as hyperplasia. A subject currently with prostate cancer has one or more of the symptoms of the disease and may have been diagnosed with prostate cancer.

As used herein, the terms treatment, treat, or treating refers to a method of reducing the effects of a disease or condition (e.g., prostate cancer) or symptom of the disease or condition. Thus, in the disclosed method, treatment can refer to a 10%, 20%, 30%, 40%, 50%, 60%, 70%, 80%, 90%, or 100% reduction in the severity of an established disease or condition or symptom of the disease or condition. For example, a method of treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100% or any percent reduction between 10 and 100% as compared to native or control levels. It is understood that treatment does not necessarily refer to a cure or complete ablation of the disease, condition, or symptoms of the disease or condition.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the materials for which they are cited are hereby specifically incorporated by reference in their entireties.

### EXAMPLES

### Example 1

### Identification of Biomarker Predictors for the Recurrence of Prostate Cancer Associated with TMPRSS2-ERG Gene Fusion

### RNA Samples.

Total RNA samples from frozen prostate tumor specimens used in this study were prepared previously (Nam et al., Br. J. Cancer 97:1690-5 (2007)). Aliquoted RNA samples were used in the cDNA-mediated annealing, selection, extension, and ligation assay (DASL assay). RNA concentration was quantified by Nanodrop spectrophotometry and quality was assessed using the Agilent Bioanalyzer (Agilent Technologies; Santa Clara, Calif.) for which RNA integrity number (RIN) of more than 7 was used as a quality criteria. DASL Assay Performance, Reporducibility, and Data Normalization.

The DASL assay was performed on Illumina's (Illumina, Inc.; San Diego, Calif.) 502-gene Human Cancer Panel (HCP) using 200 nanograms (ng) of input RNA. The manufacturer's instructions were followed without any changes. Samples were hybridized on two Universal Array Matrices (UAMs). The hybridized UAMs were scanned using the BeadStation 500 Instrument (Illumina Inc.). The data were interpreted and quantile normalized using GenomeStudio v1.0.2 (Illumina Inc.). Experimental replicates (same RNA assayed twice) were assessed for reproducibility and subsequently averaged so as to represent each patient's tumor sample with one gene expression profile.

### Data Analysis and Meta-Analysis.

Differential mRNA expression of TMPRSS2-ERG T1/E4 fusion-positive versus fusion-negative tumors was assessed using significance analysis of microarrays (SAM) (Tusher et al., Proc. Natl. Acad. Sci. USA 98:5116-21 (2001)) for which 1,000 random class assignment permutations estimated a false discovery rate (FDR) less than or equal to 5%. Hierarchical clustering was generated in R using the heatmap2 package where distance was computed using a Euclidean dissimilarity metric with an average linkage clustering algorithm. Data was displayed with mRNA intensities Z-score normalized. Gene Ontology analysis was conducted using the GOstats package with a significance value of p<0.01 of overrepresentation computed by the hypergeometric test using the lumiHumanAll.db annotation file. Univariate Cox proportional hazards regression was conducted in R using the Cox proportional hazards survival package (CoxPH) and was conducted on each gene expression profile and clinical factor independently. Multivariate Cox analysis considered clinical factors that were significant (p<0.05) in univariate analysis as well as a recurrence predictor built as a weighted average of the expression level of genes, which were significant in univariate analysis in both the Toronto data set and that from Nakagawa et al. (Nakagawa et al., PLoS ONE 3(5):e2318 (2008)). Kaplan-Meier curves were generated in R using the survival package and significance testing utilized the survdiff function for which the log-rank test determined the p-value. Meta-Analysis utilized expression profiles from both Setlur et al. (Setlur et al., J. Natl. Cancer Inst. 100(11):815-25 (2008)) and Nakagawa et al. (Nakagawa et al., PLoS ONE 3(5):e2318 (2008)) studies, which were downloaded from Gene Expression Omnibus (GEO; located on the National Center for Biotechnology Information website) and had the series numbers GSE8402 and GSE10645, respectively. The same differential, annotation, and prognostic analyses methods described above were employed on the meta-analysis sets.

### Results

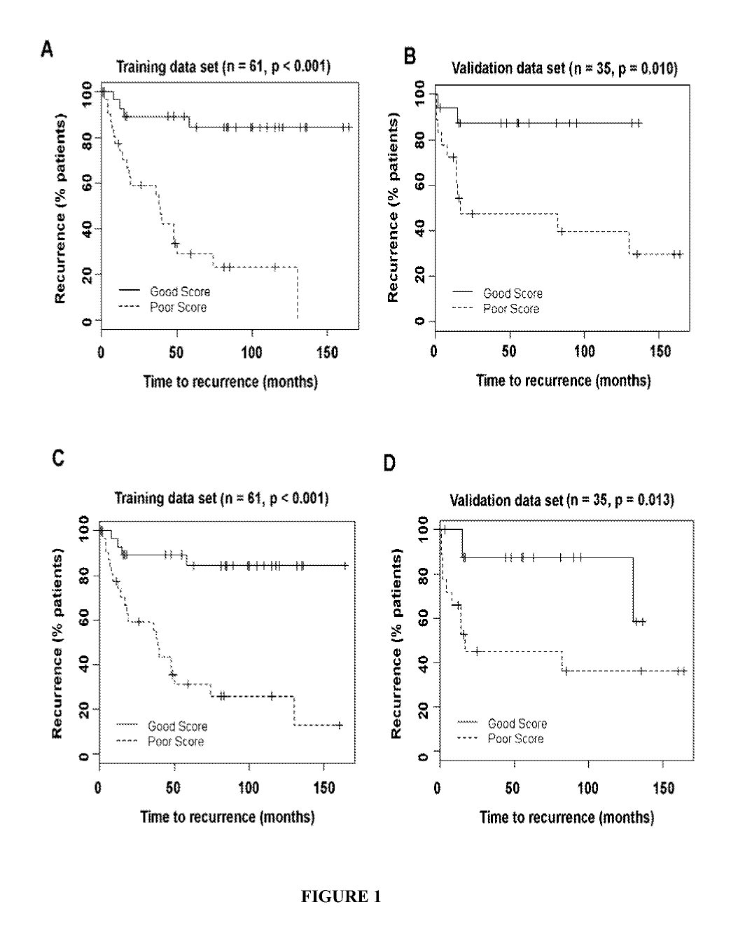

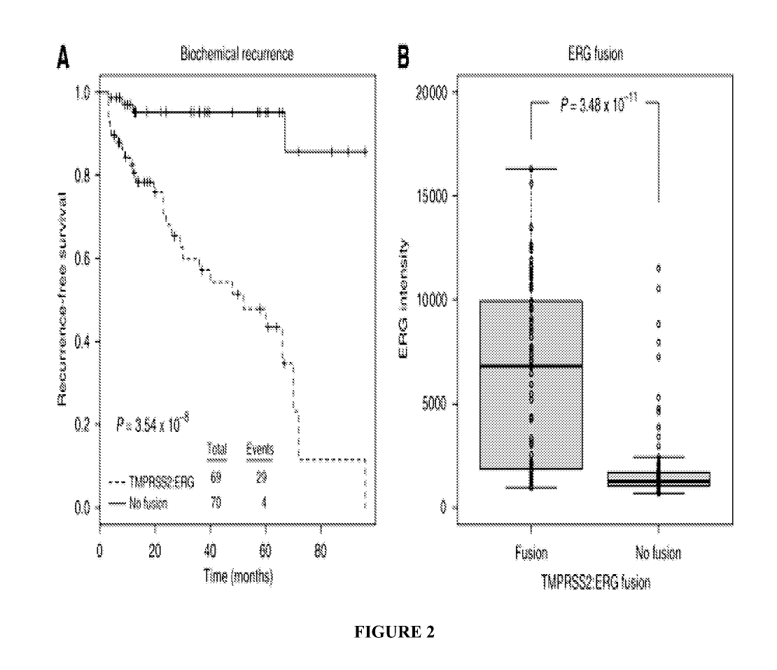

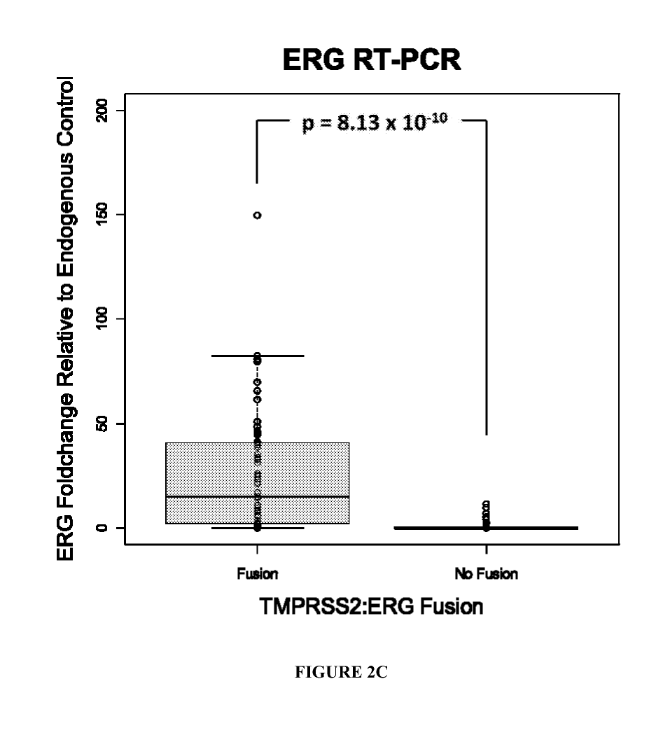

After RNA and assay quality control, 139 patient tumors were characterized on the DASL assay for 502 cancer-related genes (GEO series GSE18655). Seven samples were run as experimental replicates to estimate assay reproducibility for which an average Pearson R² of 0.965 indicated highly reproducible data (FIG. 1). Moreover, unsupervised hierarchical clustering of all samples and probes resulted in experimental replicates clustering together without exception. The Toronto cohort, a subset of that previously characterized for clinical markers (Nam et al., Br. J. Cancer 97(12):1690-5 (2007)), includes 69 patients with TMPRSS2-ERG T1/E4 fusion-positive tumors and 70 prostate tumors that were TMPRSS2-ERG fusion-negative. Fusion status indicated a significantly worse outcome with respect to biochemical recurrence (FIG. 2A, p=3.54×10⁻⁸ log-rank test) similar to that observed in the entire cohort (Nam et al., Br. J. Cancer 97(12):1690-5 (2007)). As previously reported, patients with TMPRSS2-ERG fusion-positive tumors had a significantly higher expression of ERG transcripts (FIG. 2B, p=3.48×10⁻¹¹, Student's two-sided t-test) likely a result of androgen-responsive promoter elements in TMPRSS2 driving expression (Tomlins et al., Science 310(5748):644-8 (2005)). ERG overexpression was validated using a reverse transcription-polymerase chain reaction (RT-PCR) assay, which corroborated ERG overexpression found by microarray results (FIG. 2C, p=8.13×10⁻¹⁰, Student's two-sided t-test).

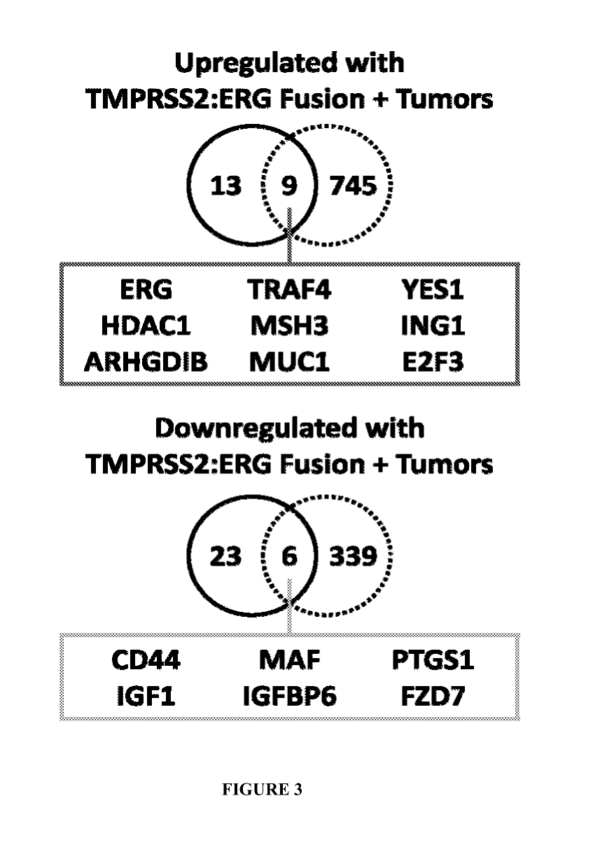

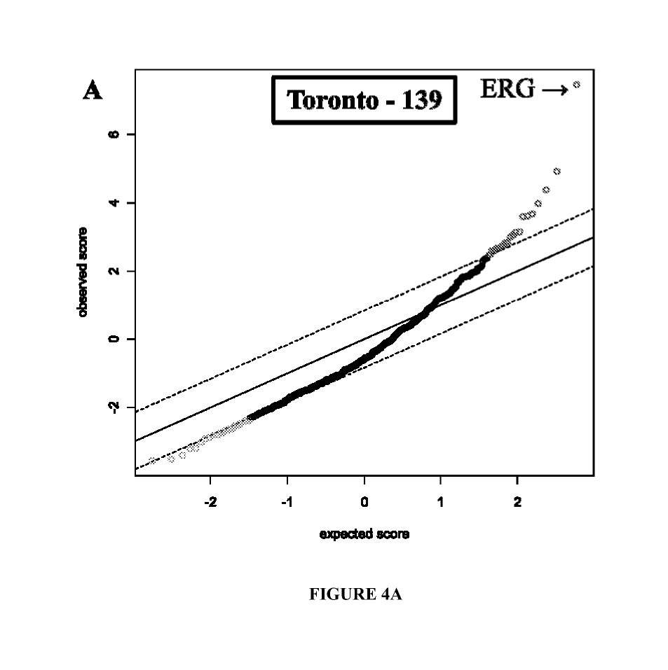

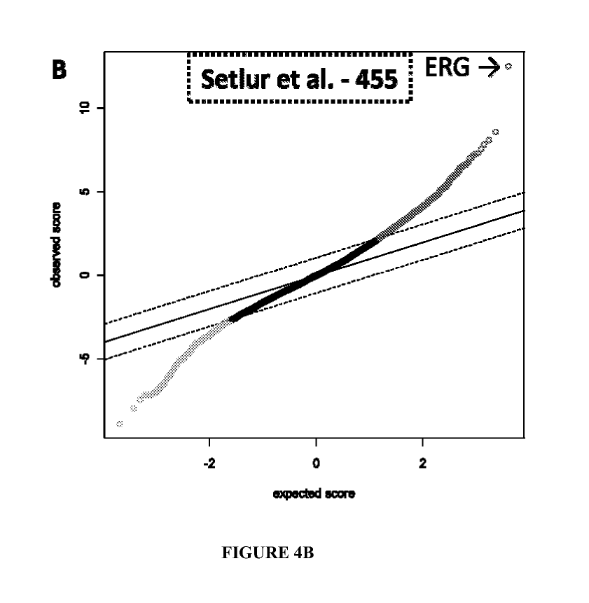

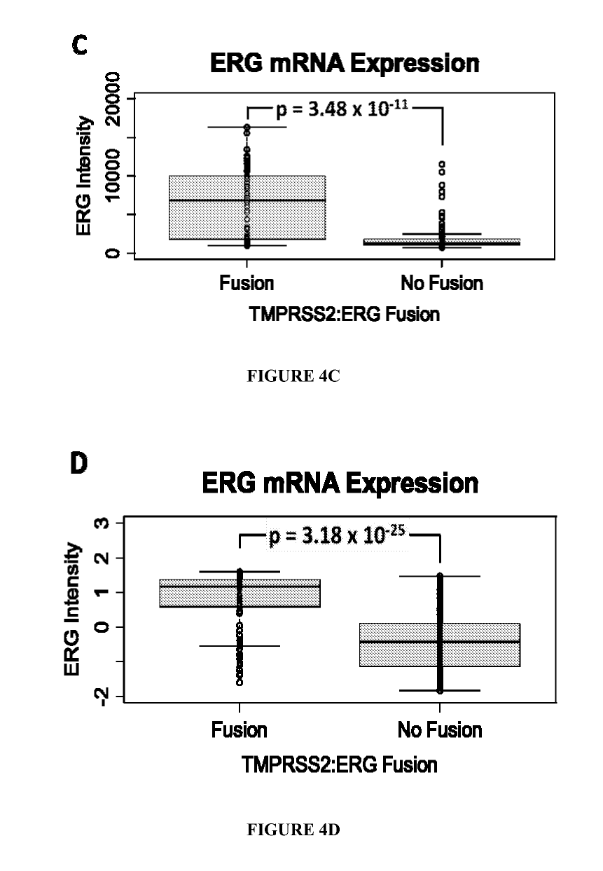

To investigate molecular biomarkers differentially regulated in TMPRSS2-ERG fusion-positive tumors, significance testing was conducted using SAM (Tusher et al., Proc. Natl. Acad. Sci. USA 98(9):5116-21 (2001)) for both the Toronto cohort and that of the 455 patient Swedish cohort (Setlur et al., J. Natl. Cancer Inst. 100(11):815-25 (2008)). Using a FDR equal to or less than 5% yielded 51 genes differentially regulated in TMPRSS2-ERG fusion-positive tumors in the Toronto cohort (Table 1). Nine upregulated genes and six downregulated genes were validated by replicating the analysis on the Swedish cohort (Setlur et al., J. Natl. Cancer Inst. 100(11):815-25 (2008)), which was characterized for expression of 6,144 transcripts (FIG. 3, FDR <5%). In both the Toronto and Swedish cohorts ERG was uniquely the most significant differentially regulated transcript in TMPRSS2-ERG fusion-positive tumors (FIG. 4). Genes annotated for mismatch base repair and histone deacetylation functions were overrepresented in Gene Ontology analysis of common upregulated genes TMPRSS2-ERG fusion positive tumors. Downregulated genes were overrepresented for annotations that included the insulin-like growth factor and Jak-Stat signaling pathways suggesting that these pathways may be attenuated in TMPRSS2-ERG fusion-positive tumors (Table 2, p<0.01). Hierarchical clustering of tumor expression profiles across common differentially regulated genes resulted in segregation of TMPRSS2-ERG fusion-positive tumors (FIG. 3), suggesting that TMPRSS2-ERG fusion-positive tumors have a distinct molecular metabolism that is replicated in multiple cohorts.

TABLE 1
Differentially regulated mRNAs with TMPRSS2-ERG fusion. Differential regulated genes
associated with TMPRSS2-ERG fusion were determined using Significance Analysis of
Microarrays (SAM) which permutated 1,000 assignments of class assignment to determine
differential targets (Tusher et al., Proc. Natl. Acad. Sci. USA 98(9): 5116-21 (2001)).
mRNAs were validated in a 455 patient Swedish cohort that was characterized for
expression of 6,144 transcripts (Setlur et al., J. Natl. Cancer Inst. 100(11): 815-25 (2008)).

| | | | | |
|---|---|---|---|---|
| | | | | FDR |
| Gene | | SAM | Fold | (q- |
| Symb. | Gene Name | Score | Change | value) |

Upregualted in TMPRSS2-ERG T1/E4 positive tumors

| | | | | |
|---|---|---|---|---|
| ERG | v-ets erythroblastosis virus E26 oncogene | 7.46 | 3.07 | 3.22% |
| | homolog | | | |
| MSF/ | septin 9 | 4.93 | 1.26 | 3.22% |
| Sept9 | | | | |
| HDAC1 | histone deacetylase 1 | 4.38 | 1.07 | 3.22% |
| EPHB4 | EPH receptor B4 | 3.99 | 1.17 | 3.22% |
| ARHGDIB | Rho GDP dissociation inhibitor (GDI) beta | 3.67 | 1.06 | 3.22% |
| THPO | Thrombopoietin | 3.62 | 1.23 | 3.22% |
| PDGFA | platelet-derived growth factor alpha polypeptide | 3.60 | 1.09 | 3.22% |
| CEACAM1 | carcinoembryonic antigen-related cell adhesion | 3.16 | 1.23 | 3.22% |
| | mol. 1 | | | |
| SHH | sonic hedgehog homolog (Drosophila) | 3.12 | 1.14 | 3.22% |
| TRAF4 | TNF receptor-associated factor 4 | 3.06 | 1.14 | 3.22% |
| IFNGR1 | interferon gamma receptor 1 | 3.00 | 1.09 | 3.22% |
| MSH3 | mutS homolog 3 (E. coli) | 2.84 | 1.10 | 3.22% |
| MUC1 | mucin 1, cell surface associated | 2.83 | 1.42 | 3.22% |
| PXN | Paxillin | 2.74 | 1.10 | 3.22% |
| ITGB4 | integrin, beta 4 | 2.71 | 1.07 | 3.22% |
| CDK4 | cyclin-dependent kinase 4 | 2.68 | 1.08 | 3.22% |
| CDK7 | cyclin-dependent kinase 7 | 2.66 | 1.08 | 3.22% |
| YES1 | v-yes-1 Yamaguchi sarcoma viral oncogene | 2.63 | 1.08 | 3.22% |
| | homolog 1 | | | |
| ING1 | inhibitor of growth family, member 1 | 2.59 | 1.08 | 3.22% |
| E2F3 | E2F transcription factor 3 | 2.59 | 1.16 | 3.22% |
| WT1 | Wilms tumor 1 | 2.51 | 1.16 | 4.80% |
| SOD1 | superoxide dismutase 1, soluble | 2.49 | 1.02 | 4.80% |

Downregulated in TMPRSS2-ERG T1/E4 positive tumors

| | | | | |
|---|---|---|---|---|
| CD44 | CD44 molecule (Indian blood group) | −3.56 | −1.12 | 3.22% |
| LAF4/ | AF4/FMR2 family, member 3 | −3.51 | −1.28 | 3.22% |
| AFF3 | | | | |
| EPO | erythropoietin | −3.41 | −1.28 | 3.22% |
| KDR | kinase insert domain receptor (a type III rec. tyr. | −3.20 | −1.14 | 3.22% |
| | kin.) | | | |
| GFI1 | growth factor independent 1 transcription | −3.19 | −1.10 | 3.22% |
| | repressor | | | |
| FGF12 | fibroblast growth factor 12 | −3.02 | −1.39 | 3.22% |
| FGFR4 | fibroblast growth factor receptor 4 | −2.91 | −1.14 | 3.22% |
| PTEN | phosphatase and tensin homolog | −2.87 | −1.11 | 3.22% |
| FLT4 | fms-related tyrosine kinase 4 | −2.83 | −1.14 | 3.22% |
| IGF1 | insulin-like growth factor 1 (somatomedin C) | −2.81 | −1.11 | 3.22% |
| FLT1 | fms-related tyrosine kinase 1 (vegf/vpfr) | −2.80 | −1.16 | 3.22% |
| TGFBR1 | transforming growth factor, beta receptor 1 | −2.74 | −1.09 | 3.22% |
| EXT1 | exostoses (multiple) 1 | −2.73 | −1.14 | 3.22% |
| TNFSF6 | Fas ligand (TNF superfamily, member 6) | −2.67 | −1.06 | 3.22% |
| TGFB3 | transforming growth factor, beta 3 | −2.66 | −1.15 | 3.22% |
| FGF7 | fibroblast growth factor 7 (keratinocyte growth | −2.64 | −1.20 | 3.22% |
| | factor) | | | |
| PDGFRA | platelet-derived growth factor receptor, alpha | −2.64 | −1.22 | 3.22% |
| | polypeptide | | | |
| MAF | v-maf musculoaponeurotic fibrosarc. onc. | −2.61 | −1.15 | 3.22% |
| | homolog | | | |
| IGF2 | insulin-like growth factor 2 (somatomedin A) | −2.53 | −1.38 | 3.22% |
| WNT2B | wingless-type MMTV integration site family, | −2.53 | −1.26 | 3.22% |
| | member 2B | | | |
| NOTCH4 | Notch homolog 4 (Drosophila) | −2.52 | −1.15 | 3.22% |
| ETV1 | ets variant 1 | −2.48 | −1.55 | 5.00% |
| IGFBP6 | insulin-like growth factor binding protein 6 | −2.48 | −1.11 | 5.00% |
| CBL | Cas-Br-M (murine) ecotropic retroviral | −2.42 | −1.12 | 5.00% |
| | transforming seq. | | | |
| PTGS1 | prostaglandin-endoperoxide synthase 1 | −2.39 | −1.14 | 5.00% |
| FZD7 | frizzled homolog 7 (Drosophila) | −2.39 | −1.11 | 5.00% |
| FYN | FYN oncogene related to SRC, FGR, YES | −2.39 | −1.14 | 5.00% |
| PLAG1 | pleiomorphic adenoma gene 1 | −2.38 | −1.10 | 5.00% |
| L1CAM | L1 cell adhesion molecule | −2.38 | −1.08 | 5.00% |

TABLE 2
Gene Ontology Annotation of mRNAs associated with TMPRSS2-ERG T1/E4 fusion
in Prostate Cancer. The 15 mRNAs associated with TMPRSS2-ERG T1/E4 fusion (FIG. 3,
Table 1 in bold) in both our Toronto-139 cohort and a Swedish 455 patient cohort (Setlur et
al., J. Natl. Cancer Inst. 100(11): 815-25 (2008)) were annotated for gene ontology terms.
Several terms annotated from the nine upregulated mRNAs in T1/E4 fusion positive tumors
were related to DNA damage & repair mechanisms and histone deacetylation. Conversly,
overrepresented terms for the six mRNAs downregulated in T1/E4 positive tumors were
associated with insulin-like growth factor (IGF) activity and JAK-STAT tyrosine
phosporylation signaling. P-values were calculated using a hypergeometric test in the R
package GOstats.
Ontology Annotation of Overexpressed mRNAs in TMPRSS2-ERG fusion positive tumors

| | | | P- |
|---|---|---|---|
| GOBPID | Term | Category | value |
| GO: 0043570 | maintenance of DNA repeat elements | Biological Process | 0.0011 |
| GO: 0032302 | MutSbeta complex | Cellular | 0.0011 |
| | | Component | |
| GO: 0000700 | mismatch base pair DNA N-glycosylase | Molecular | 0.0011 |
| | activity | Function | |
| GO: 0000701 | purine-specific mismatch base pair DNA N- | Molecular | 0.0011 |
| | glycosylase activity | Function | |
| GO: 0032181 | dinucleotide repeat insertion binding | Molecular | 0.0011 |
| | | Function | |
| GO: 0032300 | mismatch repair complex | Cellular | 0.0016 |
| | | Component | |
| GO: 0005094 | Rho GDP-dissociation inhibitor activity | Molecular | 0.0017 |
| | | Function | |
| GO: 0019237 | centromeric DNA binding | Molecular | 0.0017 |
| | | Function | |
| GO: 0032139 | dinucleotide insertion or deletion binding | Molecular | 0.0017 |
| | | Function | |
| GO: 0032142 | single guanine insertion binding | Molecular | 0.0017 |
| | | Function | |
| GO: 0032356 | oxidized DNA binding | Molecular | 0.0017 |
| | | Function | |
| GO: 0032357 | oxidized purine DNA binding | Molecular | 0.0017 |
| | | Function | |
| GO: 0005515 | protein binding | Molecular | 0.0021 |
| | | Function | |
| GO: 0032134 | mispaired DNA binding | Molecular | 0.0022 |
| | | Function | |
| GO: 0032135 | DNA insertion or deletion binding | Molecular | 0.0022 |
| | | Function | |
| GO: 0032137 | guanine/thymine mispair binding | Molecular | 0.0022 |
| | | Function | |
| GO: 0032138 | single base insertion or deletion binding | Molecular | 0.0022 |
| | | Function | |
| GO: 0005092 | GDP-dissociation inhibitor activity | Molecular | 0.0028 |
| | | Function | |
| GO: 0019104 | DNA N-glycosylase activity | Molecular | 0.0055 |
| | | Function | |
| GO: 0016575 | histone deacetylation | Biological Process | 0.0058 |
| GO: 0016447 | somatic recombination of immunoglob. gene | Biological Process | 0.0068 |
| | seg. | | |
| GO: 0016445 | somatic diversification of immunoglobulins | Biological Process | 0.0079 |
| GO: 0016799 | hydrolase activity, hydrolyzing N-glycosyl | Molecular | 0.0088 |
| | comp. | Function | |
| GO: 0030983 | mismatched DNA binding | Molecular | 0.0088 |
| | | Function | |
| GO: 0002562 | somatic diversification of immune receptors | Biological Process | 0.0089 |
| | via germline recombination within a single | | |
| | locus | | |
| GO: 0006476 | protein amino acid deacetylation | Biological Process | 0.0089 |
| GO: 0016444 | somatic cell DNA recombination | Biological Process | 0.0089 |
| GO: 0002200 | somatic diversification of immune receptors | Biological Process | 0.0094 |
| GO: 0002377 | immunoglobulin production | Biological Process | 0.0094 |
| GO: 0004407 | histone deacetylase activity | Molecular | 0.0099 |
| | | Function | |
| GO: 0009441 | glycolate metabolic process | Biological Process | 0.0005 |
| GO: 0014834 | satellite cell maintenance involved in | Biological Process | 0.0005 |
| | skeletal muscle regeneration | | |
| GO: 0014904 | myotube cell development | Biological Process | 0.0005 |
| GO: 0034392 | negative regulation of smooth muscle cell | Biological Process | 0.0005 |
| | apoptosis | | |
| GO: 0004666 | prostaglandin-endoperoxide synthase | Molecular | 0.0008 |
| | activity | Function | |
| GO: 0014911 | positive regulation of smooth muscle cell | Biological Process | 0.0009 |
| | migration | | |
| GO: 0033143 | regulation of steroid hormone receptor | Biological Process | 0.0009 |
| | signaling pathway | | |
| GO: 0035019 | somatic stem cell maintenance | Biological Process | 0.0009 |
| GO: 0043568 | positive regulation of insulin-like growth | Biological Process | 0.0009 |
| | factor receptor signaling pathway | | |
| GO: 0051450 | myoblast proliferation | Biological Process | 0.0009 |
| GO: 0014896 | muscle hypertrophy | Biological Process | 0.0014 |
| GO: 0043403 | skeletal muscle regeneration | Biological Process | 0.0014 |
| GO: 0016942 | insulin-like growth factor binding protein | Cellular | 0.0016 |
| | complex | Component | |
| GO: 0034390 | smooth muscle cell apoptosis | Biological Process | 0.0018 |
| GO: 0034391 | regulation of smooth muscle cell apoptosis | Biological Process | 0.0018 |
| GO: 0043500 | muscle adaptation | Biological Process | 0.0018 |
| GO: 0043567 | regulation of insulin-like growth factor | Biological Process | 0.0018 |
| | receptor signaling pathway | | |
| GO: 0014902 | myotube differentiation | Biological Process | 0.0023 |
| GO: 0042523 | positive regulation of tyrosine | Biological Process | 0.0023 |
| | phosphorylation of Stat5 protein | | |
| GO: 0019827 | stem cell maintenance | Biological Process | 0.0027 |
| GO: 0042522 | regulation of tyrosine phosphorylation of | Biological Process | 0.0027 |
| | Stat5 protein | | |
| GO: 0048864 | stem cell development | Biological Process | 0.0027 |
| GO: 0014909 | smooth muscle cell migration | Biological Process | 0.0032 |
| GO: 0014910 | regulation of smooth muscle cell migration | Biological Process | 0.0032 |
| GO: 0042506 | tyrosine phosphorylation of Stat5 protein | Biological Process | 0.0032 |
| GO: 0045821 | positive regulation of glycolysis | Biological Process | 0.0032 |
| GO: 0042813 | Wnt receptor activity | Molecular | 0.0033 |
| | | Function | |
| GO: 0014065 | phosphoinositide 3-kinase cascade | Biological Process | 0.0036 |
| GO: 0048863 | stem cell differentiation | Biological Process | 0.0036 |
| GO: 0001516 | prostaglandin biosynthetic process | Biological Process | 0.0041 |
| GO: 0014812 | muscle cell migration | Biological Process | 0.0041 |
| GO: 0046457 | prostanoid biosynthetic process | Biological Process | 0.0041 |
| GO: 0046579 | positive regulation of Ras protein signal | Biological Process | 0.0041 |
| | transduction | | |
| GO: 0032787 | monocarboxylic acid metabolic process | Biological Process | 0.0042 |
| GO: 0006110 | regulation of glycolysis | Biological Process | 0.0045 |
| GO: 0051057 | positive regulation of small GTPase | Biological Process | 0.0045 |
| | mediated signal transduction | | |
| GO: 0004926 | non-G-protein coupled 7TM receptor | Molecular | 0.0046 |
| | activity | Function | |
| GO: 0005159 | insulin-like growth factor receptor binding | Molecular | 0.0046 |
| | | Function | |
| GO: 0031331 | positive regulation of cellular catabolic | Biological Process | 0.0050 |
| | process | | |
| GO: 0042246 | tissue regeneration | Biological Process | 0.0050 |
| GO: 0042531 | positive regulation of tyrosine | Biological Process | 0.0050 |
| | phosphorylation of STAT protein | | |
| GO: 0043470 | regulation of carbohydrate catabolic process | Biological Process | 0.0050 |
| GO: 0043471 | regulation of cellular carbohydrate catabolic | Biological Process | 0.0050 |
| | process | | |
| GO: 0048009 | insulin-like growth factor receptor signaling | Biological Process | 0.0050 |
| | pathway | | |
| GO: 0046427 | positive regulation of JAK-STAT cascade | Biological Process | 0.0054 |
| GO: 0048661 | positive regulation of smooth muscle cell | Biological Process | 0.0054 |
| | prolif. | | |
| GO: 0040007 | Growth | Biological Process | 0.0056 |
| GO: 0031099 | Regeneration | Biological Process | 0.0058 |
| GO: 0045913 | positive regulation of carbohydrate | Biological Process | 0.0058 |
| | metabolic process | | |
| GO: 0065008 | regulation of biological quality | Biological Process | 0.0064 |
| GO: 0006692 | prostanoid metabolic process | Biological Process | 0.0067 |
| GO: 0006693 | prostaglandin metabolic process | Biological Process | 0.0067 |
| GO: 0045740 | positive regulation of DNA replication | Biological Process | 0.0067 |
| GO: 0048146 | positive regulation of fibroblast proliferation | Biological Process | 0.0067 |
| GO: 0005518 | collagen binding | Molecular | 0.0070 |
| | | Function | |
| GO: 0005540 | hyaluronic acid binding | Molecular | 0.0070 |
| | | Function | |
| GO: 0009896 | positive regulation of catabolic process | Biological Process | 0.0072 |
| GO: 0031329 | regulation of cellular catabolic process | Biological Process | 0.0076 |
| GO: 0042509 | regulation of tyrosine phosphorylation of | Biological Process | 0.0076 |
| | STAT prot. | | |
| GO: 0045840 | positive regulation of mitosis | Biological Process | 0.0076 |
| GO: 0048144 | fibroblast proliferation | Biological Process | 0.0076 |
| GO: 0048145 | regulation of fibroblast proliferation | Biological Process | 0.0076 |
| GO: 0050679 | positive regulation of epithelial cell | Biological Process | 0.0076 |
| | proliferation | | |
| GO: 0006109 | regulation of carbohydrate metabolic process | Biological Process | 0.0081 |
| GO: 0005158 | insulin receptor binding | Molecular | 0.0087 |
| | | Function | |
| GO: 0046425 | regulation of JAK-STAT cascade | Biological Process | 0.0094 |
| GO: 0005520 | insulin-like growth factor binding | Molecular | 0.0095 |
| | | Function | |
| GO: 0007260 | tyrosine phosphorylation of STAT protein | Biological Process | 0.0099 |
| GO: 0030166 | proteoglycan biosynthetic process | Biological Process | 0.0099 |
| GO: 0048660 | regulation of smooth muscle cell | Biological Process | 0.0099 |
| | proliferation | | |

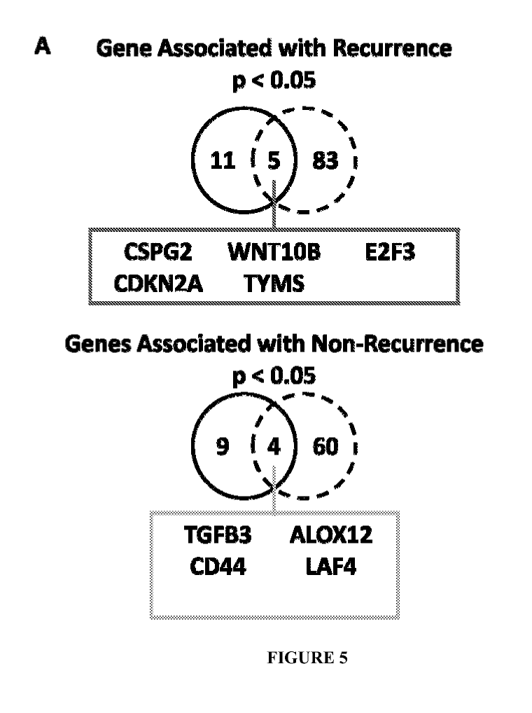

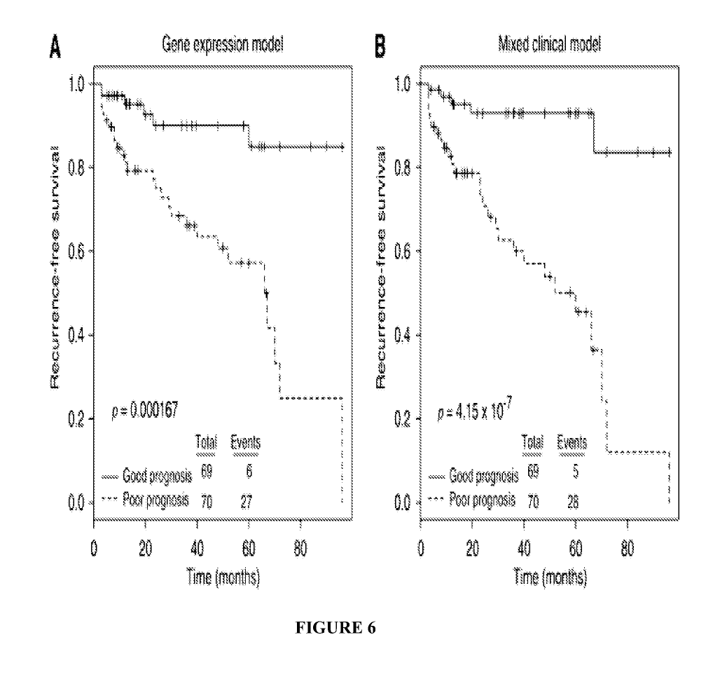

To determine molecular factors associated with biochemical recurrence, defined as a PSA increase of ≧0.2 ng/ml on at least two consecutive measurements that are at least 3 months apart, univariate Cox proportional hazards regression was conducted in the Toronto cohort and replicated in a 596 patient Minnesota cohort (Nakagawa et al., PLoS ONE 3(5):e2318 (2008)). The Toronto dataset yielded 16 genes associated with recurrence and 11 genes associated with non-recurrence (Table 3, p<0.05). Repeating this analysis in the Minnesota cohort validated five genes associated with biochemical recurrence (CSPG2, WNT10B, E2F3, CDKN2A, and TYMS) and four genes associated with non-recurrence (TGFB3, ALOX12, CD44, and LAF4) (FIG. 5, p<0.05). Gene Ontology functional annotation of genes commonly associated with recurrence yielded overrepresentation of deoxyribosylthymine monophosphate (dTMP) biosynthesis, negative regulation of leukocyte activation, specifically T and B cell lymphocytes, as well as inhibition of cell-matrix adhesion. Conversely, annotation of genes associated with non-recurrence resulted in cell-matrix adhesion and collagen binding (Table 4, p<0.01). Common genes prognostic of recurrence were used to build a recurrence score calculated as the sum product of each gene's expression intensity by its Cox coefficient determined by regression analysis. Ordering samples by the recurrence score in a supervised heatmap produced a trend whereby patients that did not have recurrence were separated from those who did in both the Toronto and Swedish cohorts. More importantly, the recurrence score was significant in univariate Cox regression and remained significant in a multivariate model considering clinical factors that were significant (p<0.05) in the univariate analysis, namely pre-operative PSA level, Gleason score, and TMPRSS2-ERG fusion status (Table 5, Toronto cohort). Furthermore, the nine-gene expression recurrence score was significantly associated with biochemical recurrence by itself (FIG. 6A, p=0.000167) and in a multivariate model considering with Gleason score and TMPRSS2-ERG fusion status (i.e., those clinical data significant in univariate analysis; FIG. 6B, p=4.15×10⁻⁷).

TABLE 3
mRNAs Associated with Biochemical Recurrence. mRNAs
associated with biochemical recurrence were determined
using a cox proportional hazards regression of mRNA
expression. mRNAs were validated in a 596 Minnesota
cohort characterized for the same 502 mRNA transcripts
(Nakagawa et al., 2008).

| | | | |
|---|---|---|---|
| Gene Symbol/ | | Cox | Cox p- |
| Alias | Gene Name | Coef. | value |

mRNAs Associated with Recurrence

| | | | |
|---|---|---|---|
| MUC1 | mucin 1, cell surface associated | 0.0003 | 0.0001 |
| CDKN2A | cyclin-dependent kinase inhibitor 2A | 0.0004 | 0.0005 |
| WNT10B | wingless-type MMTV integration site | 0.0027 | 0.0030 |
| | family, member 10B | | |
| CSPG2/ | versican | 0.0004 | 0.0057 |
| VCAN | | | |
| MSF/SEPT9 | septin 9 | 0.0003 | 0.0087 |
| E2F3 | E2F transcription factor 3 | 0.0010 | 0.0120 |
| CDH11 | cadherin 11, type 2, OB-cadherin | 0.0008 | 0.0120 |
| | (osteoblast) | | |
| MMP7 | matrix metallopeptidase 7 | 0.0001 | 0.0130 |
| | (matrilysin, uterine) | | |
| ERG | v-ets erythroblastosis virus E26 | 0.0001 | 0.0150 |
| | oncogene homolog (avian) | | |
| SKIL | SKI-like oncogene | 0.0001 | 0.0170 |
| TYMS | thymidylate synthetase | 0.0002 | 0.0220 |
| BIRC3 | baculoviral IAP repeat-containing 3 | 0.0002 | 0.0220 |
| EPHB4 | EPH receptor B4 | 0.0013 | 0.0280 |
| TNFRSF6/ | Fas (TNF receptor superfamily, | 0.0001 | 0.0300 |
| FAS | member 6) | | |
| TGFBI | transforming growth factor, beta- | 0.0002 | 0.0380 |
| | induced, 68 kDa | | |
| LCN2 | lipocalin 2 | 0.0001 | 0.0380 |

mRNAs Associated with Non-Recurrence

| | | | |
|---|---|---|---|
| CD44 | CD44 molecule (Indian blood group) | −0.0002 | 0.0092 |
| VEGF/ | vascular endothelial growth factor A | −0.0002 | 0.0170 |
| VEGFA | | | |
| EPO | erythropoietin | −0.0010 | 0.0180 |
| ALOX12 | arachidonate 12-lipoxygenase | −0.0049 | 0.0180 |
| TGFB3 | transforming growth factor, beta 3 | −0.0004 | 0.0190 |
| FLT1/ | fms-related tyrosine kinase 1 | −0.0005 | 0.0250 |
| VEGFR | (VEGF/VPFR) | | |
| FGFR4 | fibroblast growth factor receptor 4 | −0.0006 | 0.0280 |
| TYRO3 | TYRO3 protein tyrosine kinase | −0.0014 | 0.0290 |
| MAF | v-maf musculoaponeurotic | −0.0002 | 0.0310 |
| | fibrosarcoma oncogene homolog | | |
| FHIT | fragile histidine triad gene | −0.0005 | 0.0380 |
| LAF4/AFF3 | AF4/FMR2 family, member 3 | −0.0002 | 0.0400 |

TABLE 4
Gene Ontology Annotation of mRNAs associated with Biochemical Recurrence.
The nine mRNAs associated with biochemical recurrence in both the Toronto-
139 and Minnesota-596 (Nakagawa et al., 2008) were cohorts (FIG. 5, Table 3)
were annotated for gene ontology terms. Several terms were found overrepresented
in the five mRNAs associated with recurrence including deoxyribosylthymine
monophosphate (dTMP) metabolism, negative regulation of B and T-cell leukocyte
proliferation, and negative regulation of cell adhesion. Overrepresented terms for
the four mRNAs associated with non-recurrence T1/E4 positive tumors were
associated with cell adhesion and hydrolase and oxide activity. P-values were
calculated using a hypergeometric test in the R package GOstats.

| | | | |
|---|---|---|---|
| GOBPID | Term | Category | P-value |

Gene Ontology Annotation of Genes Associated with Recurrence

| | | | |
|---|---|---|---|
| GO: 0004799 | thymidylate synthase activity | Molecular | 0.0003459 |
| | | Function | |
| GO: 0042083 | 5,10-methylenetetrahydrofolate-dependent | Molecular | 0.0003459 |
| | methyltransferase activity | Function | |
| GO: 0055103 | ligase regulator activity | Molecular | 0.0003459 |
| | | Function | |
| GO: 0055104 | ligase inhibitor activity | Molecular | 0.0003459 |
| | | Function | |
| GO: 0055105 | ubiquitin-protein ligase inhibitor activity | Molecular | 0.0003459 |
| | | Function | |
| GO: 0055106 | ubiquitin-protein ligase regulator activity | Molecular | 0.0003459 |
| | | Function | |
| GO: 0006231 | dTMP biosynthetic process | Biological | 0.0003758 |
| | | Process | |
| GO: 0009157 | deoxyribonucleoside monophosphate | Biological | 0.0003758 |
| | biosynthetic process | Process | |
| GO: 0009162 | deoxyribonucleoside monophosphate | Biological | 0.0003758 |
| | metabolic process | Process | |
| GO: 0009176 | pyrimidine deoxyribonucleoside | Biological | 0.0003758 |
| | monophosphate metabolic process | Process | |
| GO: 0009177 | pyrimidine deoxyribonucleoside | Biological | 0.0003758 |
| | monophosphate biosynthetic process | Process | |
| GO: 0010149 | Senescence | Biological | 0.0003758 |
| | | Process | |
| GO: 0010389 | regulation of G2/M transition of mitotic | Biological | 0.0003758 |
| | cell cycle | Process | |
| GO: 0046073 | dTMP metabolic process | Biological | 0.0003758 |
| | | Process | |
| GO: 0030889 | negative regulation of B cell proliferation | Biological | 0.0007515 |
| | | Process | |
| GO: 0033079 | immature T cell proliferation | Biological | 0.0007515 |
| | | Process | |
| GO: 0033080 | immature T cell proliferation in the thymus | Biological | 0.0007515 |
| | | Process | |
| GO: 0033083 | regulation of immature T cell proliferation | Biological | 0.0007515 |
| | | Process | |
| GO: 0033084 | regulation of immature T cell prolif. in the | Biological | 0.0007515 |
| | thymus | Process | |
| GO: 0033087 | negative regulation of immature T cell | Biological | 0.0007515 |
| | proliferation | Process | |
| GO: 0033088 | negative regulation of immature T cell | Biological | 0.0007515 |
| | proliferation in the thymus | Process | |
| GO: 0009129 | pyrimidine nucleoside monophosphate | Biological | 0.0011271 |
| | metabolic process | Process | |
| GO: 0009130 | pyrimidine nucleoside monophosphate | Biological | 0.0011271 |
| | biosynthetic process | Process | |
| GO: 0009221 | pyrimidine deoxyribonucleotide | Biological | 0.0015026 |
| | biosynthetic process | Process | |
| GO: 0017145 | stem cell division | Biological | 0.0015026 |
| | | Process | |
| GO: 0048103 | somatic stem cell division | Biological | 0.0015026 |
| | | Process | |
| GO: 0009263 | deoxyribonucleotide biosynthetic process | Biological | 0.001878 |
| | | Process | |
| GO: 0032088 | negative regulation of NF-kappaB | Biological | 0.0022533 |
| | transcription factor activity | Process | |
| GO: 0001953 | negative regulation of cell-matrix adhesion | Biological | 0.0026284 |
| | | Process | |
| GO: 0050869 | negative regulation of B cell activation | Biological | 0.0030035 |
| | | Process | |
| GO: 0004861 | cyclin-dependent protein kinase inhibitor | Molecular | 0.0031101 |
| | activity | Function | |
| GO: 0005578 | proteinaceous extracellular matrix | Cellular Comp. | 0.0033002 |
| GO: 0031012 | extracellular matrix | Cellular Comp. | 0.0034427 |
| GO: 0030888 | regulation of B cell proliferation | Biological | 0.0037532 |
| | | Process | |
| GO: 0042130 | negative regulation of T cell proliferation | Biological | 0.0037532 |
| | | Process | |
| GO: 0045736 | neg. regulation of cyclin-dependent prot. | Biological | 0.0037532 |
| | kin. act. | Process | |
| GO: 0009219 | pyrimidine deoxyribonucleotide metabolic | Biological | 0.0041279 |
| | process | Process | |
| GO: 0001952 | regulation of cell-matrix adhesion | Biological | 0.0045025 |
| | | Process | |
| GO: 0030291 | protein serine/threonine kinase inhibitor | Molecular | 0.0048346 |
| | activity | Function | |
| GO: 0032945 | negative regulation of mononuclear cell | Biological | 0.0048769 |
| | prolif. | Process | |
| GO: 0033077 | T cell differentiation in the thymus | Biological | 0.0048769 |
| | | Process | |
| GO: 0050672 | negative regulation of lymphocyte | Biological | 0.0048769 |
| | proliferation | Process | |
| GO: 0016538 | cyclin-dependent protein kinase regulator | Molecular | 0.0051792 |
| | activity | Function | |
| GO: 0006309 | DNA fragmentation during apoptosis | Biological | 0.0052513 |
| | | Process | |
| GO: 0005540 | hyaluronic acid binding | Molecular | 0.0058681 |
| | | Function | |
| GO: 0008637 | apoptotic mitochondrial changes | Biological | 0.0059997 |
| | | Process | |
| GO: 0042100 | B cell proliferation | Biological | 0.0059997 |
| | | Process | |
| GO: 0050868 | negative regulation of T cell activation | Biological | 0.0059997 |
| | | Process | |
| GO: 0051059 | NF-kappaB binding | Molecular | 0.0062124 |
| | | Function | |
| GO: 0000086 | G2/M transition of mitotic cell cycle | Biological | 0.0063737 |
| | | Process | |
| GO: 0043433 | negative regulation of transcription factor | Biological | 0.0063737 |
| | activity | Process | |
| GO: 0009262 | deoxyribonucleotide metabolic process | Biological | 0.0067476 |
| | | Process | |
| GO: 0006921 | cell structure disassembly during apoptosis | Biological | 0.0071214 |
| | | Process | |
| GO: 0007223 | Wnt receptor signal. path., calc. modul. | Biological | 0.0071214 |
| | path. | Process | |
| GO: 0022411 | cellular component disassembly | Biological | 0.0071214 |
| | | Process | |
| GO: 0042326 | negative regulation of phosphorylation | Biological | 0.0071214 |
| | | Process | |
| GO: 0010563 | negative regulation of phosphorus | Biological | 0.0074951 |
| | metabolic process | Process | |
| GO: 0030262 | apoptotic nuclear changes | Biological | 0.0074951 |
| | | Process | |
| GO: 0045936 | negative regulation of phosphate metabolic | Biological | 0.0074951 |
| | process | Process | |
| GO: 0043392 | negative regulation of DNA binding | Biological | 0.0078687 |
| | | Process | |
| GO: 0006221 | pyrimidine nucleotide biosynthetic process | Biological | 0.0082421 |
| | | Process | |
| GO: 0051100 | negative regulation of binding | Biological | 0.0082421 |
| | | Process | |
| GO: 0007568 | aging | Biological | 0.0086155 |
| | | Process | |
| GO: 0009123 | nucleoside monophosphate metabolic | Biological | 0.0086155 |
| | process | Process | |
| GO: 0009124 | nucleoside monophosphate biosynthetic | Biological | 0.0086155 |
| | process | Process | |
| GO: 0051250 | negative regulation of lymphocyte | Biological | 0.0086155 |
| | activation | Process | |
| GO: 0004860 | protein kinase inhibitor activity | Molecular | 0.0093071 |
| | | Function | |
| GO: 0002695 | negative regulation of leukocyte activation | Biological | 0.0093618 |
| | | Process | |
| GO: 0031647 | regulation of protein stability | Biological | 0.0097348 |
| | | Process | |
| GO: 0050864 | regulation of B cell activation | Biological | 0.0097348 |
| | | Process | |
| GO: 0019210 | kinase inhibitor activity | Molecular | 0.0099937 |
| | | Function | |

Gene Ontology Annotation of Genes Prognostic of Non-Recurrence

| | | | |
|---|---|---|---|
| GO: 0004052 | arachidonate 12-lipoxygenase activity | Molecular | 0.0004151 |
| | | Function | |
| GO: 0047977 | hepoxilin-epoxide hydrolase activity | Molecular | 0.0004151 |
| | | Function | |
| GO: 0016803 | ether hydrolase activity | Molecular | 0.0010376 |
| | | Function | |
| GO: 0042554 | superoxide release | Biological | 0.0013525 |
| | | Process | |
| GO: 0016165 | lipoxygenase activity | Molecular | 0.0014524 |
| | | Function | |
| GO: 0030307 | positive regulation of cell growth | Biological | 0.0015778 |
| | | Process | |
| GO: 0016801 | hydrolase activity, acting on ether bonds | Molecular | 0.0016598 |
| | | Function | |
| GO: 0045793 | positive regulation of cell size | Biological | 0.0020282 |
| | | Process | |
| GO: 0045785 | positive regulation of cell adhesion | Biological | 0.0033789 |
| | | Process | |
| GO: 0042383 | sarcolemma | Cellular Comp. | 0.0034219 |
| GO: 0005518 | collagen binding | Molecular | 0.0035248 |
| | | Function | |
| GO: 0005540 | hyaluronic acid binding | Molecular | 0.0035248 |
| | | Function | |
| GO: 0006801 | superoxide metabolic process | Biological | 0.0036039 |
| | | Process | |
| GO: 0019370 | leukotriene biosynthetic process | Biological | 0.0040537 |
| | | Process | |
| GO: 0043450 | alkene biosynthetic process | Biological | 0.0040537 |
| | | Process | |
| GO: 0006691 | leukotriene metabolic process | Biological | 0.0049531 |
| | | Process | |
| GO: 0043449 | cellular alkene metabolic process | Biological | 0.0049531 |
| | | Process | |
| GO: 0045927 | positive regulation of growth | Biological | 0.0049531 |
| | | Process | |
| GO: 0046456 | icosanoid biosynthetic process | Biological | 0.0063011 |
| | | Process | |
| GO: 0007155 | cell adhesion | Biological | 0.0077585 |
| | | Process | |
| GO: 0022610 | biological adhesion | Biological | 0.0077585 |
| | | Process | |
| GO: 0019395 | fatty acid oxidation | Biological | 0.0078722 |
| | | Process | |
| GO: 0034440 | lipid oxidation | Biological | 0.0078722 |
| | | Process | |
| GO: 0006690 | icosanoid metabolic process | Biological | 0.0089934 |
| | | Process | |

TABLE 5
Clinical and Molecular Factors for the Toronto-139. Cohort clinical characteristics
for the 139 prostate cancer patients in the Toronto cohort are listed out for
TMPRSS2-ERG T1/E4 fusion positive and fusion negative patients. Factors were
assessed for their association with biochemical recurrence when relevant (indicated
by a univariate p-value). Factors prognostic of recurrence (p < 0.05) were used in a
multivariate model of recurrence. The nine-gene recurrence score (composed of the
genes listed in FIG. 5) is composed of mRNAs replicated as prognostic of recurrence
in this experiment and a 596 patient Minnesota experiment (Nakagawa et al., 2008).

| | | TMPRSS2-ERG | | Recurrence Model | |
|---|---|---|---|---|---|
| | | T1/E4 fusion | | (p) | |
| | Total | positive | negative | Univariate | Multi. |
| Cohort Size (n) | 139 | 69 | 70 | — | — |
| Biochemical Recurrence | 33 | 29 | 4 | — | — |
| Average Follow-up | 30.9 | 25.8 | 36 | — | — |
| (months) | | | | | |
| Avg. Age (yrs) | 61.7 | 61.1 | 62.2 | 0.0880 | — |
| Preoperative PSA (ng/mL) | | | | 0.0210 | 0.6200 |
| Average | 8.9 | 9.3 | 8.5 | | |
| Range | [2.2-43.0] | [3.4-38.9] | [2.2-43.0] | | |
| Gleason Score | | | | 0.0190 | 0.0280 |
| 5-6 | 38 | 19 | 19 | | |
| | (27.3%) | (27.5%) | (27.1%) | | |
| 7 | 90 | 46 | 44 | | |
| | (64.7%) | (66.7%) | (62.9%) | | |
| 8-9 | 11 | 4 | 2 | | |
| | (7.9%) | (5.8%) | (10.0%) | | |
| Pathologic Stage | | | | 0.0860 | — |
| organ confined | 59 | 29 | 30 | | |
| | (42.4%) | (42.0%) | (42.9%) | | |
| extraprostatic extension | 70 | 35 | 35 | | |
| | (50.4%) | (50.7%) | (50.0%) | | |
| seminal vesicle invasion | 10 | 5 | 5 | | |
| | (7.2%) | (7.2%) | (7.1%) | | |
| Positive Margin | | | | 0.4000 | — |
| No | 62 | 33 | 29 | | |
| | (44.6%) | (47.8%) | (41.4%) | | |
| Yes | 77 | 36 | 41 | | |
| | (55.4%) | (52.2%) | (58.6%) | | |
| TMPRSS2-ERG Fusion | — | — | — | | 0.0004 |
| Nine-gene Recurrence | 2.01 | 3.37 | 1.58 | | 0.0270 |
| Score [95% CI] | | [0.37, | [−0.94, | | |
| | | 7.18] | 4.25] | | |

### Example 2

### Identification of Biomarker Predictors for the Progression and Metastatic Potential of Prostate Cancer

### RNA Isolation.

RNA is isolated from formalin-fixed paraffin-embedded (FFPE) tissue according to the methods described in Abramovitz et al., Biotechniques 44(3):417-23 (2008). In brief, three 5 μm sections per block were cut and placed into a 1.5 mL sterile microfuge tube. The tissue section was deparaffinized with 100% xylene for 3 minutes at 50° C. The tissue section was centrifuged, washed twice with ethanol, and allowed to air dry. The tissue section was digested with Proteinase K for 24 hours at 50° C. RNA was isolated using an Ambion Recover All Kit (Ambion; Austin, Tex.).

cDNA-Mediated Annealing, Selection, Extension, and Ligation Assay (DASL Assay).

Upon the completion of RNA isolation, the isolated RNA is used in the DASL assay. The DASL assay is performed according to the protocols supplied by the manufacturer (Illumina, Inc.; San Diego, Calif.). The primer sequences for the fourteen biomarker genes are shown in Table 6. The probe sequences for the fourteen biomarker genes are shown in Table 7.

TABLE 6
DASL assay Primer Sequences for Fourteen Biomarker Genes

| Gene | Primer Sequences |
|---|---|
| FOXO1A | 5′-ACTTCGTCAGTAACGGACGTCCTAGGAGAAGAGCTGCATCCA-3′ |
| | (SEQ ID NO: 1) |
| | 5′-GAGTCGAGGTCATATCGTGTCCTAGGAGAAGAGCTGCATCCA-3′ |
| | (SEQ ID NO: 2) |
| SOX9 | 5′-ACTTCGTCAGTAACGGACGCTCCTACCCGCCCATCACCC-3′ |
| | (SEQ ID NO: 3) |
| | 5′-GAGTCGAGGTCATATCGTGCTCCTACCCGCCCATCACCC-3′ |
| | (SEQ ID NO: 4) |
| | 5′-ACTTCGTCAGTAACGGACGGAGAGAACTTGGTGCCTCTTCC-3′ |
| | (SEQ ID NO: 5) |
| CLNS1A | 5′-GAGTCGAGGTCATATCGTGGAGAGAACTTGGTGCCTCTTCC-3′ |
| | (SEQ ID NO: 6) |
| | 5′-ACTTCGTCAGTAACGGACGCGAACCCAGACCCCCAGG-3′ |
| | (SEQ ID NO: 7) |
| PTGDS | 5′-GAGTCGAGGTCATATCGTGCGAACCCAGACCCCCAGG-3′ |
| | (SEQ ID NO: 8) |
| | 5′-ACTTCGTCAGTAACGGACGCCAGCAAAGAATGGCTCAAGAA-3′ |
| | (SEQ ID NO: 9) |
| XPO1 | 5′-GAGTCGAGGTCATATCGTGCCAGCAAAGAATGGCTCAAGAA-3′ |
| | (SEQ ID NO: 10) |
| | 5′-ACTTCGTCAGTAACGGACGTCACCTTTCTCCAAAGGCAGATG-3′ |
| | (SEQ ID NO: 11) |
| LETMD | 5′-GAGTCGAGGTCATATCGTGTCACCTTTCTCCAAAGGCAGATG-3′ |
| | (SEQ ID NO: 12) |
| RAD23B | 5′-ACTTCGTCAGTAACGGACAATCCTTCCTTGCTTCCAGCG-3′ |
| | (SEQ ID NO: 13) |
| | 5′-GAGTCGAGGTCATATCGTAATCCTTCCTTGCTTCCAGCG-3′ |
| | (SEQ ID NO: 14) |
| TMPRSS | 5′-ACTTCGTCAGTAACGGACAGCGCGGCACTCAGGTACCT-3′ |
| | (SEQ ID NO: 15) |
| 2_ETV1 | 5′-ACTTCGTCAGTAACGGACAGCGCGGCACTCAGGTACCT-3′ |
| FUSION | (SEQ ID NO: 16) |
| ABCC3 | 5′-ACTTCGTCAGTAACGGACATGTTCCTGTGCTCCATGATGC-3′ |
| | (SEQ ID NO: 17) |
| | 5′-GAGTCGAGGTCATATCGTATGTTCCTGTGCTCCATGATGC-3′ |
| | (SEQ ID NO: 18) |
| | 5′-GTCGCTGATCTTACAACACTATTACATGCCTATTGACGTGAGGCGGTCTG |
| | CCTATAGTGAGTC-3′ |
| | (SEQ ID NO: 19) |
| APC | 5′-ACTTCGTCAGTAACGGACGTCCCTGGAGTAAAACTGCGGTC-3′ |
| | (SEQ ID NO: 20) |
| | 5′-GAGTCGAGGTCATATCGTGTCCCTGGAGTAAAACTGCGGTC-3′ |
| | (SEQ ID NO: 21) |
| | 5′-AAAATGTCCCTCCGTTCTTATCTAGATCGCAAAAGTGTCTCGGAAGTCTG |
| | CCTATAGTGAGTC-3′ |
| | (SEQ ID NO: 22) |
| CHES1 | 5′-ACTTCGTCAGTAACGGACGGGTTTCTCCAAGGCCCTTCA-3′ |
| | (SEQ ID NO: 23) |
| | 5′-GAGTCGAGGTCATATCGTGGGTTTCTCCAAGGCCCTTCA-3′ |
| | (SEQ ID NO: 24) |
| | 5′-GAAGACGATGACCTCGACTTCATACGCGAATTGATAGAAGCTCGGTCTG |
| | CCTATAGTGAGTC-3′ |
| | (SEQ ID NO: 25) |
| EDNRA | 5′-ACTTCGTCAGTAACGGACTGCAACTCTGCTCAGGATCATTT-3′ |
| | (SEQ ID NO: 26) |
| | 5′-GAGTCGAGGTCATATCGTTGCAACTCTGCTCAGGATCATTT-3′ |
| | (SEQ ID NO: 27) |
| | 5′-CCAGAACAAATGTATGAGGAATTCACTCAAGGCCGTTAGCTGTGGTCTG |
| | CCTATAGTGAGTC-3′ |
| | (SEQ ID NO: 28) |
| FRZB | 5′-ACTTCGTCAGTAACGGACGGAAGCTTCGTCATCTTGGACTCAG-3′ |
| | (SEQ ID NO: 29) |
| | 5′-GAGTCGAGGTCATATCGTGGAAGCTTCGTCATCTTGGACTCAG-3′ |
| | (SEQ ID NO: 30) |
| | 5′-AAAAGTGATTCTAGCAATAGTGATTTTACTGCGCTCCTAATTGGCACCGT |
| | CTGCCTATAGTGAGTC-3′ |
| | (SEQ ID NO: 31) |
| HSPG2 | 5′-ACTTCGTCAGTAACGGACCCAAATGCGCTGGACACATT-3′ |
| | (SEQ ID NO: 32) |
| | 5′-GAGTCGAGGTCATATCGTCCAAATGCGCTGGACACATT-3′ |
| | (SEQ ID NO: 33) |
| | 5′-GTACCTTTCTGATGATGAGGACGGAACAGCTTACGACTTTGCGGGTCTG |
| | CCTATAGTGAGTC-3′ |
| | (SEQ ID NO: 34) |

TABLE 7
Probe Sequences for Detection of Fourteen Biomarker
Genes in DASL assay

| Gene | Probe Sequence |
|---|---|
| FOXO1A | 5′-TCCTAGGAGAAGAGCTGCATCCATGGACAACAACAGTAAATTTGCTA- |
| | 3′ |
| | (SEQ ID NO: 35) |
| SOX9 | 5′-CTCCTACCCGCCCATCACCCGCTCACAGTACGACTACACCGAC-3′ |
| | (SEQ ID NO: 36) |
| CLNS1A | 5′-GGAGAGAACTTGGTGCCTCTTCCACTCTGGAGTGAAGTTAATGA |
| | AAG-3′ |
| | (SEQ ID NO: 37) |
| PTGDS | 5′-CGAACCCAGACCCCCAGGGCTGAGTTAAAGGAGAAATTCACC-3′ |
| | (SEQ ID NO: 38) |
| XPO1 | 5′-CCAGCAAAGAATGGCTCAAGAAGTACTGACACATTTAAAGGAGCAT- |
| | 3′ |
| | (SEQ ID NO: 39) |
| LETMD1 | 5′-TCACCTTTCTCCAAAGGCAGATGTGAAGAACTTGATGTCTTATGTGG- |
| | 3′ |
| | (SEQ ID NO: 40) |
| RAD23B | 5′-AATCCTTCCTTGCTTCCAGCGTTACTACAGCAGATAGGTCGAGAG-3′ |
| | (SEQ ID NO: 41) |
| TMPRSS2_ | 5′-AGCGCGGCACTCAGGTACCTGACAATGATGAGCAGTTTGTACC-3′ |
| ETV1 | (SEQ ID NO: 42) |
| FUSION | |
| ABCC3 | 5′-ATGTTCCTGTGCTCCATGATGCAGTCGCTGATCTTACAACACTATT-3′ |
| | (SEQ ID NO: 43) |
| APC | 5′-TCCCTGGAGTAAAACTGCGGTCAAAAATGTCCCTCCGTTCTTAT-3′ |
| | (SEQ ID NO: 44) |
| CHES1 | 5′-GGTTTCTCCAAGGCCCTTCAGGAAGACGATGACCTCGACTT-3′ |
| | (SEQ ID NO: 45) |
| EDRNA | 5′-TGCAACTCTGCTCAGGATCATTTACCAGAACAAATGTATGAGGAAT-3′ |
| | (SEQ ID NO: 46) |
| FRZB | 5′-GAAGCTTCGTCATCTTGGACTCAGTAAAAGTGATTCTAGCAATAGTG |
| | ATT-3′ |
| | (SEQ ID NO: 47) |
| HSPG2 | 5′-CCAAATGCGCTGGACACATTCGTACCTTTCTGATGATGAGGAC-3′ |
| | (SEQ ID NO: 48) |

For each of the genes in the predictive nine-gene score, the signal is obtained by the average of three probes. The sets of DASL assay primer sequences are given in Table 8, and the DASL probe sequences are given in Table 9.

TABLE 8
DASL assay Primer Sequences for Nine Biomarker Genes

| Gene | Primer Sequences |
|---|---|
| ALOX12 | 5′-ACTTCGTCAGTAACGGACGTTACGCTTTGCAGACCGCATAG-3′ |
| | (SEQ ID NO: 49) |
| | 5′-GAGTCGAGGTCATATCGTGTTACGCTTTGCAGACCGCATAG-3′ |
| | (SEQ ID NO: 50) |
| ALOX12 | 5′-ACTTCGTCAGTAACGGACGATCGCTGCAGACCGTAAGGATG-3′ |
| | (SEQ ID NO: 51) |
| | 5′-GAGTCGAGGTCATATCGTGATCGCTGCAGACCGTAAGGATG-3′ |
| | (SEQ ID NO: 52) |
| ALOX12 | 5′-ACTTCGTCAGTAACGGACCTAAGGCTCTATTTCCTCCCCCA-3′ |
| | (SEQ ID NO: 53) |
| | 5′-GAGTCGAGGTCATATCGTCTAAGGCTCTATTTCCTCCCCCA-3′ |
| | (SEQ ID NO: 54) |
| CD44 | 5′-ACTTCGTCAGTAACGGACCACCCGCTATGTCCAGAAAGGA-3′ |
| | (SEQ ID NO: 55) |
| | 5′-GAGTCGAGGTCATATCGTCACCCGCTATGTCCAGAAAGGA-3′ |
| | (SEQ ID NO: 56) |
| CD44 | 5′-ACTTCGTCAGTAACGGACGCTAATCCCTGGGCATTGCTTTC-3′ |
| | (SEQ ID NO: 57) |
| | 5′-GAGTCGAGGTCATATCGTGCTAATCCCTGGGCATTGCTTTC-3′ |
| | (SEQ ID NO: 58) |
| CD44 | 5′-ACTTCGTCAGTAACGGACCAGCTGATGAGACAAGGAACCTG-3′ |
| | (SEQ ID NO: 59) |
| | 5′-GAGTCGAGGTCATATCGTCAGCTGATGAGACAAGGAACCTG-3′ |
| | (SEQ ID NO: 60) |
| CDKN2A | 5′-ACTTCGTCAGTAACGGACGGGAAGCTGTCGACTTCATGACAAG-3′ |
| | (SEQ ID NO: 61) |
| | 5′-GAGTCGAGGTCATATCGTGGGAAGCTGTCGACTTCATGACAAG-3′ |
| | (SEQ ID NO: 62) |
| CDKN2A | 5′-ACTTCGTCAGTAACGGACGAACCCACCCCGCTTTCGTA-3′ |
| | (SEQ ID NO: 63) |
| | 5′-GAGTCGAGGTCATATCGTGAACCCACCCCGCTTTCGTA-3′ |
| | (SEQ ID NO: 64) |
| CDKN2A | 5′-ACTTCGTCAGTAACGGACGCGCTTCTGCCTTTTCACTGTGTT-3′ |
| | (SEQ ID NO: 65) |
| | 5′-GAGTCGAGGTCATATCGTGCGCTTCTGCCTTTTCACTGTGTT-3′ |
| | (SEQ ID NO: 66) |
| CSPG2 | 5′-ACTTCGTCAGTAACGGACCCACAGTCCAACCTCAGGCTATC-3′ |
| | (SEQ ID NO: 67) |
| | 5′-GAGTCGAGGTCATATCGTCCACAGTCCAACCTCAGGCTATC-3′ |
| | (SEQ ID NO: 68) |
| CSPG2 | 5′-ACTTCGTCAGTAACGGACGCATGGAAGGAAGTGCTTTGGG-3′ |
| | (SEQ ID NO: 69) |
| | 5′-GAGTCGAGGTCATATCGTGCATGGAAGGAAGTGCTTTGGG-3′ |
| | (SEQ ID NO: 70) |
| CSPG2 | 5′-ACTTCGTCAGTAACGGACTGCTCCAGAGTACAACTGGCGT-3′ |
| | (SEQ ID NO: 71) |
| | 5′-GAGTCGAGGTCATATCGTTGCTCCAGAGTACAACTGGCGT-3′ |
| | (SEQ ID NO: 72) |
| E2F3 | 5′-ACTTCGTCAGTAACGGACGCTCAGGATGGGGTCAGATGGAG-3′ |
| | (SEQ ID NO: 73) |
| | 5′-GAGTCGAGGTCATATCGTGCTCAGGATGGGGTCAGATGGAG-3′ |
| | (SEQ ID NO: 74) |
| E2F3 | 5′-ACTTCGTCAGTAACGGACTAAGTTGGACCAAGGGAAGTCGG-3′ |
| | (SEQ ID NO: 75) |
| | 5′-GAGTCGAGGTCATATCGTTAAGTTGGACCAAGGGAAGTCGG-3′ |
| | (SEQ ID NO: 76) |
| E2F3 | 5′-ACTTCGTCAGTAACGGACAGGTTTATCAGCCTCTGCAAGGA-3′ |
| | (SEQ ID NO: 77) |
| | 5′-GAGTCGAGGTCATATCGTAGGTTTATCAGCCTCTGCAAGGA-3′ |
| | (SEQ ID NO: 78) |
| LAF4 | 5′-ACTTCGTCAGTAACGGACTCCTCTAACAAGTGGCAGCTGGA-3′ |
| | (SEQ ID NO: 79) |
| | 5′-GAGTCGAGGTCATATCGTTCCTCTAACAAGTGGCAGCTGGA-3′ |
| | (SEQ ID NO: 80) |
| LAF4 | 5′-ACTTCGTCAGTAACGGACGGGAGATCAAGAAGTCCCAGGG-3′ |
| | (SEQ ID NO: 81) |
| | 5′-GAGTCGAGGTCATATCGTGGGAGATCAAGAAGTCCCAGGG-3′ |
| | (SEQ ID NO: 82) |
| LAF4 | 5′-ACTTCGTCAGTAACGGACGTCTGATCCAAAATGAAAGCCACG-3′ |
| | (SEQ ID NO: 83) |
| | 5′-GAGTCGAGGTCATATCGTGTCTGATCCAAAATGAAAGCCACG-3′ |
| | (SEQ ID NO: 84) |
| TGFB3 | 5′-ACTTCGTCAGTAACGGACGAGGGGATGGGGATAGAGGAAAG-3′ |
| | (SEQ ID NO: 85) |
| | 5′-GAGTCGAGGTCATATCGTGAGGGGATGGGGATAGAGGAAAG-3′ |
| | (SEQ ID NO: 86) |
| TGFB3 | 5′-ACTTCGTCAGTAACGGACGCATGTCACACCTTTCAGCCCAAT-3′ |
| | (SEQ ID NO: 87) |
| | 5′-GAGTCGAGGTCATATCGTGCATGTCACACCTTTCAGCCCAAT-3′ |
| | (SEQ ID NO: 88) |
| TGFB3 | 5′-ACTTCGTCAGTAACGGACCGGTGGTAAAGAAAGTGTGGGTTT-3′ |
| | (SEQ ID NO: 89) |
| | 5′-GAGTCGAGGTCATATCGTCGGTGGTAAAGAAAGTGTGGGTTT-3′ |
| | (SEQ ID NO: 90) |
| TYMS | 5′-ACTTCGTCAGTAACGGACGGGTGCTTTCAAAGGAGCTTGAA-3′ |
| | (SEQ ID NO: 91) |
| | 5′-GAGTCGAGGTCATATCGTGGGTGCTTTCAAAGGAGCTTGAA-3′ |
| | (SEQ ID NO: 92) |
| TYMS | 5′-ACTTCGTCAGTAACGGACTTGACACCATCAAAACCAACCC-3′ |
| | (SEQ ID NO: 93) |
| | 5′-GAGTCGAGGTCATATCGTTTGACACCATCAAAACCAACCC-3′ |
| | (SEQ ID NO: 94) |
| TYMS | 5′-ACTTCGTCAGTAACGGACAGGGATCCACAAATGCTAAAGAGC-3′ |
| | (SEQ ID NO: 95) |
| | 5′-GAGTCGAGGTCATATCGTAGGGATCCACAAATGCTAAAGAGC-3′ |
| | (SEQ ID NO: 96) |
| WNT10B | 5′-ACTTCGTCAGTAACGGACCCACCCCTCTTCTGCTCCTTAGA-3′ |
| | (SEQ ID NO: 97) |
| | 5′-GAGTCGAGGTCATATCGTCCACCCCTCTTCTGCTCCTTAGA-3′ |
| | (SEQ ID NO: 98) |
| WNT10B | 5′-ACTTCGTCAGTAACGGACGCTGTCCAGGCCCTTAGGGAAGT-3′ |
| | (SEQ ID NO: 99) |
| | 5′-GAGTCGAGGTCATATCGTGCTGTCCAGGCCCTTAGGGAAGT-3′ |
| | (SEQ ID NO: 100) |
| WNT10B | 5′-ACTTCGTCAGTAACGGACTGCTGTGTGATGAGTGCAAGGTTA-3′ |
| | (SEQ ID NO: 101) |
| | 5′-GAGTCGAGGTCATATCGTTGCTGTGTGATGAGTGCAAGGTTA-3′ |
| | (SEQ ID NO: 102) |

TABLE 9
Probe Sequences for Detection of Nine Biomarker Genes in DASL assay

| Gene | Probe Sequence |
|---|---|
| ALOX12 | 5′-CACTGTCTCAACTACTCAGCTCTCCTGATACGCGAGCCTAGACGTGTCTGCCT |
| | ATA GTGAGTC-3′ |
| | (SEQ ID NO: 103) |
| ALOX12 | 5′-TCTACCTCCAAATATGAGATTCCTGTAGCCCTACGCGACGGTTGAGTCTGCC |
| | TATAG TGAGTC-3′ |
| | (SEQ ID NO: 104) |
| ALOX12 | 5′-TTAAACCCCCTACATTAGTATCCTACACAGCGACCGTACCATCGTGTCTGCC |
| | TATAG TGAGTC-3′ |
| | (SEQ ID NO: 105) |
| CD44 | 5′-AATACAGAACGAATCCTGAAGACAAAGCCGATCTTCGCCCAGTCTGTCTGC |
| | CTATAG TGAGTC-3′ |
| | (SEQ ID NO: 106) |
| CD44 | 5′-ACTGAGGTTGGGGTGTACTAGTAAGGGTGCGACACTATCTCGACGTCTGCC |
| | TATAGT GAGTC-3′ |
| | (SEQ ID NO: 107) |
| CD44 | 5′-AGAATGTGGACATGAAGATTGGTTCTAATGGGCGCACCAAACCGTCTGCCT |
| | ATAGTG AGTC-3′ |
| | (SEQ ID NO: 108) |
| CDKN2 | 5′-ATTTTGTGAACTAGGGAAGCTCGCCTGGCGAATAAAGGTCGTACGTCTGCC |
| A | TATAGT GAGTC-3′ |
| | (SEQ ID NO: 109) |
| CDKN2 | 5′-TTTTCATTTAGAAAATAGAGCTTTTCGTTACATCCATCGCAGCGACGTCTGC |
| A | CTATAG TGAGTC-3′ |
| | (SEQ ID NO: 110) |
| CDKN2 | 5′-GAGTTTTCTGGAGTGAGCACTAATTGGGTCTCGCAGTAGTGGCGTCTGCCTA |
| A | TAGTG AGTC-3′ |
| | (SEQ ID NO: 111) |
| CSPG2 | 5′-CAGATAGTTTAGCCACCAAATTAAACGATGTCCGTGATTGCCTGGGTCTGCC |
| | TATAG TGAGTC-3′ |
| | (SEQ ID NO: 112) |
| CSPG2 | 5′-GAAGTAGAAGATGTGGACCTCTCCAAATAGGCCGTGTCCTCCGTGGTCTGC |
| | CTATAG TGAGTC-3′ |
| | (SEQ ID NO: 113) |
| CSPG2 | 5′-TCTCATTATGCTACGGATTCATTAGGGTTCGGGTTCAGACACCGGTCTGCCT |
| | ATAGTG AGTC-3′ |
| | (SEQ ID NO: 114) |
| E2F3 | 5′-GACCTCTAGGGAGAAAGACATCACCTATTTGGCGGAGGACCACTGTCTGCC |
| | TATAGT GAGTC-3′ |
| | (SEQ ID NO: 115) |
| E2F3 | 5′-GACGTAAAAAATGAAGCAAAACTAGCTGGCCCACGAAATCTGCGGTCTGCC |
| | TATAG TGAGTC-3′ |
| | (SEQ ID NO: 116) |
| E2F3 | 5′-CTTTGTCCCATCGTGCTTCAGAGCTGCACCCGACTTGGTCAGTCTGCCTATA |
| | GTGAGTC-3′ |
| | (SEQ ID NO: 117) |
| LAF4 | 5′-AAATGGCTAAACAAAGTTAATCCGCCGGTAATGCTATGCTGACTCGTCTGCC |
| | TATAG TGAGTC-3′ |
| | (SEQ ID NO: 118) |
| LAF4 | 5′-GAGAAAGACAGCTCTTCAAGACTCGTAGTGATGCAGATGCGCTGTGTCTGC |
| | CTATAG TGAGTC-3′ |
| | (SEQ ID NO: 119) |
| LAF4 | 5′-GTCAGAGAGCAATCAGTACTACAAGCCCGGCATAATACAGTCCTACGTCTG |
| | CCTATA GTGAGTC-3′ |
| | (SEQ ID NO: 120) |
| TGFB3 | 5′-GATGGTAAGTTGAGATGTTGTGTTTGAGTCGAAGATAGCCAATCACGGTCT |
| | GCCTAT AGTGAGTC-3′ |
| | (SEQ ID NO: 121) |
| TGFB3 | 5′-GAGATATCCTGGAAAACATTCACGATTGGGTACAATTCGGCTCTAGGGTCT |
| | GCCTAT AGTGAGTC-3′ |
| | (SEQ ID NO: 122) |
| TGFB3 | 5′-GTTAGAGGAAGGCTGAACTCTTTGTTAGCATCAGGTTCGTCTAAGGGTCTGC |
| | CTATA GTGAGTC-3′ |
| | (SEQ ID NO: 123) |
| TYMS | 5′-GATATTGTCAGTCTTTAGGGGTTTGCTACAGATGATGCCGAGAAGAGGTCTG |
| | CCTAT AGTGAGTC-3′ |
| | (SEQ ID NO: 124) |
| TYMS | 5′-GAC GACAGAAGAATCATCATGTCACTCCTCAGATTAGCCGAGATAAGTCTG |
| | CCTATA GTGAGTC-3′ |
| | (SEQ ID NO: 125) |
| TYMS | 5′-GTCTTCCAAGGGAGTGAAAATTGCGTAGAATAGCTGCTCATATCGGTCTGCC |
| | TATAG TGAGTC-3′ |
| | (SEQ ID NO: 126) |
| WNT10B | 5′-ACCTGAATGGACTAAGATGAAATGAACTTATGGATTTCACGAGGGCAGTCT |
| | GCCTAT AGTGAGTC-3′ |
| | (SEQ ID NO: 127) |
| WNT10B | 5′-GTCTCCTTCCATTCAGATGTTATCCGAGGACCTTACTTTAGCAGAAGTCTGC |
| | CTATAG TGAGTC-3′ |
| | (SEQ ID NO: 128) |
| WNT10B | 5′-AGAGTGGGTGAATGTGTGTAAGCTTCCGTACTGTTACAATGTGCGCGTCTGC |
| | CTATA GTGAGTC-3′ |
| | (SEQ ID NO: 129) |

To compute the predictive nine-gene score, DASL signal levels are quantile normalized across the array and the signal for each of the three probes is averaged to produce a gene signal. The nine-gene score is then computed using the following formula:

NINE GENE SCORE=(C_{CSPG2}×CSPG2_{AvgGeneSignal})+(C_{CDKN2A}×CDKN2A_{AvgGeneSignal})+(C_{WNT10B}×WNT10B_{AvgGeneSignal})+(C_{TYMS}×TYMS_{AvgGeneSignal})+(C_{E2F3}×E2F3_{AvgGeneSignal})+(C_{LAF4}×LAF4_{AvgGeneSignal})+(C_{ALOX12}×ALOX12_{AvgGeneSignal})+(C_{CD44}×CD44_{AvgGeneSignal})+(C_{TGFB3}×TGFB3_{AvgGeneSignal}).

The coefficients for the predictive nine-gene score are as follows: C_{CSPG2}=0.000295, C_{CDKN2A}=0.00024, C_{WNT10B}=0.001528, C_{TYMS}=0.000219 C_{E2F3}=0.000585, C_{LAF4}=−8.8e-05, C_{ALOX12}=−0.00291, C_{CD44}=−0.00012, C_{TGFB3}=−0.00025.

To compute the predictive fourteen-gene score, DASL signal levels are quantile normalized across the array, and then Z-score normalized across the samples. (Z-score=(signal−average(signal))/stdev(signal)). Once the predictive scores are computed, samples are separated based on whether they are greater or less than the median score. If a sample has a score greater than the median, the subject is predicted to not have recurrence. If the score is less than the median, the subject is predicted to have recurrence. For this predictive score, the higher the score, the less likely the subject is to have recurrence.

The predictive fourteen-gene score can be calculated using the following formula:

FOURTEEN GENE SCORE=(C_{FOXO1A}×FOXO1A_{Zscore})+(C_{SOX9}×SOX9_{Zscore})+(C_{CLNS1A}×CLNS1A_{Zscore})+(C_{PTGDS}×PTGDS_{Zscore})+(C_{XPO1}×XPO1_{Zscore})+(C_{RAD23B}×RAD23B_{Zscore})+(C_{TMPRSS2}_{_{—}}_{ETV1 FUSION}×TMPRSS2_{—}ETV1 FUSION_{Zscore})+(C_{ABCC3}×ABCC3_{Zscore})+(C_{APC}×APC_{Zscore})+(C_{CHES1}×CHES1_{Zscore})+(C_{EDNRA}×EDNRA_{Zscore})+(C_{FRZB}×FRZB_{Zscore})+(C_{HSPG2} X HSPG2_{Zscore}).

The coefficients for the predictive fourteen-gene score are as follows: C_{FOXO1A}=0.687, C_{SOX9}=0.351, C_{CLNS1A}=0.112, C_{PTGDS}=0.058, C_{XPO1}=−0.208, C_{LETMD1}=−0.019, C_{RAD23B}=−0.065, C_{TMPRSS2}_{_{—}}_{ETV1 FUSION}=−0.168, C_{ABCC3}=−0.202, C_{APC}=−0.128, C_{FRZB}=0.310, C_{HSPG2}=−0.048, C_{EDNRA}=0.539, and C_{CHES1}=−0.143.

The coefficients for the predictive seven-gene score are as follows: C_{FOXO1A}=0.625, C_{SOX9}=0.253, C_{CLNS1A}=0.0, C_{PTGDS}=0.056, C_{XPO1}=−0.092, C_{LETMD1}=−0.140, C_{RAD23B}=−0.045, and C_{TMPRSS2}_{_{—}}_{ETV1 FUSION}=−0.137.

miRNA Expression Profiling

The isolated RNA is additionally used in the Illumina Human Version 2 MicroRNA Expression Profiling kit (Illumina, Inc.; San Diego, Calif.) in conjunction with the DASL assay. The miRNA expression profiling is performed according to the manufacturer's protocol. The mature miRNA sequence for the six miRNA biomarkers are shown in Table 10. The probe sequences for the six miRNA biomarkers are shown in Table 11.

TABLE 10
Mature miRNA Sequences for Six
miRNA Biomarkers

| Gene | Mature miRNA sequence |
|---|---|
| Hsa-miR-103 | 5′-AGCAGCATTGTACAGGGCTATGA-3′ |
| | (SEQ ID NO: 130) |
| Hsa-miR-339 | 5′-TCCCTGTCCTCCAGGAGCTCA-3′ |
| | (SEQ ID NO: 131) |
| Hsa-miR-183 | 5′-TATGGCACTGGTAGAATTCACTG-3′ |
| | (SEQ ID NO: 132) |
| Hsa-miR-182 | 5′-TTTGGCAATGGTAGAACTCACA-3′ |
| | (SEQ ID NO: 133) |
| Hsa-miR-136 | 5′-AGCTACATTGTCTGCTGGGTTTC-3′ |
| | (SEQ ID NO: 134) |
| Hsa-miR-221 | 5′-ACTCCATTTGTTTTGATGATGGA-3′ |
| | (SEQ ID NO: 135) |

TABLE 11
Probe Sequences for Detection of Six miRNA
Biomarker Genes in DASL assay

| Gene | Probe Sequence |
|---|---|
| Hsa-miR-103 | 5′-ACTTCGTCAGTAACGGACTCCAGTAGCGACTAGCCCGTCAGCAG |
| | CATTGTACAGGGCTA-3′ |
| | (SEQ ID NO: 136) |
| Hsa-miR-339 | 5′-ACTTCGTCAGTAACGGACTATACCGGCCTAAGCACTCGCACCC |
| | TGTCCTCCAGGAGCT-3′ |
| | (SEQ ID NO: 137) |
| Hsa-miR-183 | 5′-ACTTCGTCAGTAACGGACAATGTTGACCCGGATCTCGTCCATGG |
| | CACTGGTAGAATTCA-3′ |
| | (SEQ ID NO: 138) |
| Hsa-miR-182 | 5′-ACTTCGTCAGTAACGGACACTAGCCCTCGCATAGCTTGCGTTTG |
| | GCAATGGTAGAACTC-3′ |
| | (SEQ ID NO: 139) |
| Hsa-miR-136 | 5′-ACTTCGTCAGTAACGGACGCGCAATTCCCTCGATCTTACGCTA |
| | CATTGTCTGCTGGGT-3′ |
| | (SEQ ID NO: 140) |
| Hsa-miR-221 | 5′-ACTTCGTCAGTAACGGACGTAGGTCCCGGACGTAATCACCAC |
| | TCCATTTGTTTTGATGAT-3′ |
| | (SEQ ID NO: 141) |

To compute a predictive miRNA score, DASL signal levels are quantile normalized across the array, and then Z-score normalized across the samples. (Z-score=(signal−average(signal))/stdev(signal)). The more positive the predictive score, the more likely the subject will recur. The more negative the score, the less likely the patient will recur.

The predictive six miRNA gene score can be calculated using the following formula:

SIX miRNA SCORE=miR-103_{Zscore}+miR-339_{Zscore}+miR-183_{Zscore}+miR-182_{Zscore}−miR-136_{Zscore}−miR221_{Zscore}.

### Results

A highly predictive set of 520 genes was determined through analysis of multiple publicly available gene expression datasets (Dhanasekaran et al., Nature 412:822-6 (2001); Lapointe et al., Proc. Natl. Acad. Sci. USA 101:811-6 (2004); LaTulippe et al., Cancer Res. 62:4499-506 (2002); Varambally et al., Cancer Cell 8:393-406 (2005)), datasets from gene expression profiling analysis of 58 prostate cancer patient samples (Liu et al., Cancer Res. 66:4011-9 (2006)), and genes involved in prostate cancer progression based on state of the art understanding of the disease (Tomlins et al., Science 310:644-8 (2005); Varambally et al., Cancer Cell 8:393-406 (2005)). The predictive set of 520 genes were optimized for performance in the cDNA-mediated annealing, selection, extension, and ligation (DASL) assay (Illumina, Inc.; San Diego, Calif.). The DASL assay is based upon multiplexed reverse transcription-polymerase chain reaction (RT-PCR) applied in a microarray format and enables the quantitation of expression of up to 1536 probes using RNA isolated from archived formalin-fixed paraffin embedded (FFPE) tumor tissue samples in a high throughput format (Bibokova et al., Am. J. Pathol. 165:1799-807 (2004); Fan et al., Genome Res. 14:878-85 (2004)). RNA was isolated from 71 patient samples with definitive clinical outcomes and was analyzed using the DASL assay. Based on the data from 71 patients, a subset of fourteen protein encoding genes were found to be capable of separating Gleason 7 subjects with and without recurrence, and thus were found to be good predictors of recurrent, progressive, or metastatic prostate cancers. The fourteen protein encoding genes included: FOXO1A, SOX9, CLNS1A, PTGDS, XPO1, LETMD1, RAD23B, ABCC3, APC, CHES1, EDNRA, FRZB, HSPG2, and the TMPRSS2_ETV1 FUSION. The expression of CLNS1A, XPO1, LETMD1, RAD23B, ABCC3, APC, CHES1, FRZB, HSPG2, and TMPRSS2_ETV1 FUSION was increased in recurrent, progressive, or metastatic prostate cancers, while the expression of FOXO1A, SOX9, EDNRA, and PTGDS was decreased in recurrent, progressive, or metastatic prostate cancers. Additionally, based on data obtained from the 71 patients using the MicroRNA Expression Profiling Panels (Illumin, Inc.; San Diego, Calif.) designed for the DASL assay, it was found that six miRNA genes were found to be good predictors of recurrent, progressive, or metastatic prostate cancers. The six miRNA genes included: miR-103, miR-339, miR-183, miR-182, miR-136, and miR-221. The expression of miR-103, miR-339, miR-183, and miR-182 was increased in recurrent, progressive, or metastatic prostate cancers, while the expression of miR-136 and miR-221 was decreased in recurrent, progressive, or metastatic prostate cancers.

### Example 3

To identify biomarkers predictive of recurrence, FFPE tissue blocks from 73 prostatectomy patient samples were assembled to perform DASL expression profiling with our custom-designed panel of 522 prostate cancer relevant genes. This training set of samples included 29 cases with biochemical PSA recurrence, and 44 cases without recurrence. A lasso Cox PH models was fit to identify the probes that achieved the optimal prediction performance, with the tuning parameter for Lasso selected using a leave-one-out cross-validation technique. This approach identified a panel of eight protein-coding genes (CTNNA1, XPO1, PTGDS, SOX9, RELA, EPB49, SIM2, and EDNRA) that could be used to predict recurrence following radical prostatectomy.

| | | | | | Co- |
|---|---|---|---|---|---|
| | | | Test | | efficient |
| Probe Name | | | Statistics | P-values | estimate |
| GI_55770843-S | 606 | CTNNA1 | 16.62092 | 4.56E−05 | 0.001453 |
| GI_53759152-S | 732 | XPO1 | 16.33069 | 5.32E−05 | 0.255873 |
| GI_38505192-S | 2370 | PTGDS | 10.3771 | 0.001276 | −0.09241 |
| GI_37704387-S | 1730 | SOX9 | 10.2512 | 0.001366 | −0.05805 |
| GI_46430498-S | 2021 | RELA | 9.511572 | 0.002042 | −0.07655 |
| GI_4503580-S | 3030 | EPB49 | 9.280651 | 0.002316 | −0.11694 |
| GI_7108363-A | 3740 | SIM2 | 8.372014 | 0.00381 | 0.030768 |
| GI_4503464-S | 3923 | EDNRA | 7.270868 | 0.007008 | 0.07344 |

Kaplan-Meier analysis demonstrated that these probes could significantly discriminate patients with and without recurrence by the log rank test (p=1.16e-06). This predictive model was applied to a separate DASL profiling experiment on 40 prostate cancer cases (27 without recurrence and 13 with recurrence). Kaplan-Meier analysis on this validation set determined that the model could significantly discriminate patients with and without recurrence (p=0.000153).

| | | Test | | Coefficient | |
|---|---|---|---|---|---|
| SYMBOL | DEFINITION | Statistics | P-values | estimate | Description |
| CTNNA1 | Homo sapiens catenin | 16.62092 | 0.0000456 | 0.001453 | catenin (cadherin- |
| | (cadherin-associated | | | | associated protein); |
| | protein); alpha 1; | | | | alpha 1; 102 kDa |
| | 102 kDa (CTNNA1); | | | | (CTNNA1); mRNA. |
| | mRNA. | | | | |
| XPO1 | Homo sapiens exportin | 16.33069 | 0.0000532 | 0.255873 | exportin 1 (CRM1 |
| | 1 (CRM1 homolog; | | | | homolog; yeast) |
| | yeast) (XPO1); mRNA. | | | | (XPO1); mRNA. |
| SOX9 | Homo sapiens SRY (sex | 10.2512 | 0.001366 | −0.05805 | SRY (sex |
| | determining region Y)- | | | | determining region |
| | box 9 (campomelic | | | | Y)-box 9 |
| | dysplasia; autosomal | | | | (campomelic |
| | sex-reversal) (SOX9); | | | | dysplasia; autosomal |
| | mRNA. | | | | sex-reversal) |
| | | | | | (SOX9); mRNA. |
| RELA | Homo sapiens v-rel | 9.511572 | 0.002042 | −0.07655 | v-rel |
| | reticuloendotheliosis | | | | reticuloendotheliosis |
| | viral oncogene homolog | | | | viral oncogene |
| | A; nuclear factor of | | | | homolog A; nuclear |
| | kappa light polypeptide | | | | factor of kappa light |
| | gene enhancer in B-cells | | | | polypeptide gene |
| | 3; p65 (avian) (RELA); | | | | enhancer in B-cells 3; |
| | mRNA. | | | | p65 (avian) (RELA); |
| | | | | | mRNA. |
| PTGDS | Homo sapiens | 10.3771 | 0.001276 | −0.09241 | prostaglandin D2 |
| | prostaglandin D2 | | | | synthase 21 kDa |
| | synthase 21 kDa (brain) | | | | (brain) (PTGDS); |
| | (PTGDS); mRNA. | | | | mRNA. |
| EPB49 | Homo sapiens | 9.280651 | 0.002316 | −0.11694 | erythrocyte |
| | erythrocyte membrane | | | | membrane protein |
| | protein band 4.9 | | | | band 4.9 (dematin) |
| | (dematin) (EPB49); | | | | (EPB49); mRNA. |
| | mRNA. | | | | |
| SIM2 | Homo sapiens single- | 8.372014 | 0.00381 | 0.030768 | single-minded |
| | minded homolog 2 | | | | homolog 2 |
| | (Drosophila) (SIM2); | | | | (Drosophila) (SIM2); |
| | transcript variant SIM2; | | | | transcript variant |
| | mRNA. | | | | SIM2; mRNA. |
| EDNRA | Homo sapiens | 7.270868 | 0.007008 | 0.07344 | endothelin receptor |
| | endothelin receptor type | | | | type A (EDNRA); |
| | A (EDNRA); mRNA. | | | | mRNA. |
| FOXO1A | Homo sapiens forkhead | 7.057994 | 0.007891 | −0.00292 | forkhead box O1A |
| | box O1A | | | | (rhabdomyosarcoma) |
| | (rhabdomyosarcoma) | | | | (FOXO1A); mRNA. |
| | (FOXO1A); mRNA. | | | | |

| SYMBOL | PROBE_SEQUENCE | Oligo 1 | Oligo 2 | Oligo 3 |
|---|---|---|---|---|
| CTNNA1 | TGTCCATGCAGGC | ACTTCGTCAG | GAGTCGAGGT | CAAGTGGGATCC |
| | AACATAAACTTCA | TAACGGACGT | CATATCGTGT | TAAAAGTCTAGC |
| | AGTGGGATCCTAA | GTCCATGCAG | GTCCATGCAG | GGAAACTGGCG |
| | AAGTCTAG | GCAACATAAA | GCAACATAAA | ATCAGCTAGTGT |
| | (SEQ ID | CT | CT | CTGCCTATAGTG |
| | NO: 142) | (SEQ ID | (SEQ ID | (SEQ ID |
| | | NO: 143) | NO: 144) | AGTC |
| | | | | NO: 145) |
| XPO1 | CCAGCAAAGAATG | ACTTCGTCAG | GAGTCGAGGT | TACTGACACATT |
| | GCTCAAGAAGTAC | TAACGGACGC | CATATCGTGC | TAAAGGAGCAT |
| | TGACACATTTAAA | CAGCAAAGAA | CAGCAAAGAA | ACCCACAGACGT |
| | GGAGCAT | TGGCTCAAGA | TGGCTCAAGA | TGGTCCGTAGGT |
| | (SEQ ID | A | A | CTGCCTATAGTG |
| | NO: 146) | (SEQ ID | (SEQ ID | AGTC |
| | | NO: 147) | NO: 148) | (SEQ ID |
| | | | | NO: 149) |
| SOX9 | CTCCTACCCGCCC | ACTTCGTCAG | GAGTCGAGGT | CTCACAGTACGA |
| | ATCACCCGCTCAC | TAACGGACGC | CATATCGTGC | CTACACCGACTC |
| | AGTACGACTACAC | TCCTACCCGC | TCCTACCCGC | TGGGAGTACCTA |
| | CGAC | CCATCACCC | CCATCACCC | GCTTCGGAGTCT |
| | (SEQ ID | (SEQ ID | (SEQ ID | GCCTATAGTGAG |
| | NO: 150) | NO: 151) | NO: 152) | TC |
| | | | | (SEQ ID |
| | | | | NO: 153) |
| RELA | TCCCTTTACGTCAT | ACTTCGTCAG | GAGTCGAGGT | CCATCAACTATG |
| | CCCTGAGCACCAT | TAACGGACTC | CATATCGTTC | ATGAGTTTCCAC |
| | CAACTATGATGAG | CCTTTACGTC | CCTTTACGTC | AGGCAAGCGTG |
| | TTTCC | ATCCCTGAGC | ATCCCTGAGC | GGTCTCATGGTC |
| | (SEQ ID | SEQ ID | SEQ ID | TGCCTATAGTGA |
| | NO: 154) | NO: 155) | NO: 156) | GTC |
| | | | | (SEQ ID |
| | | | | NO: 157) |
| PTGDS | AGCACCTACTCCG | ACTTCGTCAG | GAGTCGAGGT | GAGACCGACTAC |
| | TGTCAGTGGTGGA | TAACGGACGA | CATATCGTGA | GACCAGTACCGC |
| | GACCGACTACGAC | GCACCTACTC | GCACCTACTC | TGAACGTCAAAT |
| | CAGTAC | CGTGTCAGTG | CGTGTCAGTG | TGCAGGGGTCTG |
| | (SEQ ID | GT | GT | CCTATAGTGAGT |
| | NO: 158) | (SEQ ID | (SEQ ID | C |
| | | NO: 159) | NO: 160) | (SEQ ID |
| | | | | NO: 161) |
| EPB49 | CCCTCAGACCAAG | ACTTCGTCAG | GAGTCGAGGT | AGGATCTCATCA |
| | CACCTCATCGAGG | TAACGGACCC | CATATCGTCC | TCGAGTCATATT |
| | ATCTCATCATCGA | CTCAGACCAA | CTCAGACCAA | CCAGGGGAGCT |
| | GTCAT | GCACCTCATC | GCACCTCATC | ACGAGCGTGTCT |
| | (SEQ ID | SEQ ID | SEQ ID | GCCTATAGTGAG |
| | NO: 162) | NO: 163) | NO: 164) | TC |
| | | | | (SEQ ID |
| | | | | NO: 165) |
| SIM2 | TTTGTGGTAGCAT | ACTTCGTCAG | GAGTCGAGGT | ATCATGTATATA |
| | CTGATGGCAAAAT | TAACGGACGT | CATATCGTGT | TCCGAGACCGGC |
| | CATGTATATATCC | TTGTGGTAGC | TTGTGGTAGC | CTAGTAGATCGG |
| | GAGACCG | ATCTGATGGC | ATCTGATGGC | CGCAATTTCGTC |
| | (SEQ ID | AA | AA | TGCCTATAGTGA |
| | NO: 166) | (SEQ ID | (SEQ ID | GTC |
| | | NO: 167) | NO: 168) | (SEQ ID |
| | | | | NO:1 69) |
| EDNRA | GGTGTAAAAGCAG | ACTTCGTCAG | GAGTCGAGGT | TAAGAGATATTT |
| | CACAAGTGCAATA | TAACGGACGG | CATATCGTGG | CCTCAAATTTGC |
| | AGAGATATTTCCT | TGTAAAAGCA | TGTAAAAGCA | GGACAGTACCTA |
| | CAAATTTGC | GCACAAGTGC | GCACAAGTGC | CGTTGGCAAAGG |
| | (SEQ ID | A | A | TCTGCCTATAGT |
| | NO: 170) | (SEQ ID | (SEQ ID | GAGTC |
| | | NO: 171) | NO: 172) | (SEQ ID |
| | | | | NO: 173) |
| FOXO1A | TCCTAGGAGAAGA | ACTTCGTCAG | GAGTCGAGGT | GGACAACAACA |
| | GCTGCATCCATGG | TAACGGACGT | CATATCGTGT | GTAAATTTGCTA |
| | ACAACAACAGTAA | CCTAGGAGAA | CCTAGGAGAA | TCCTGTAGTACC |
| | ATTTGCTA | GAGCTGCATC | GAGCTGCATC | GGGTTTGAAAGG |
| | (SEQ ID | CA | CA | GTCTGCCTATAG |
| | NO: 174) | (SEQ ID | (SEQ ID | TGAGTC |
| | | NO: 175) | NO: 176) | (SEQ ID |
| | | | | NO: 177) |

In addition, comprehensive DASL miRNA profiling of these same 73 FFPE cases was performed using the MicroRNA Expression Profiling Panels (Illumina, Inc.) designed for the DASL assay. MicroRNA probes were filtered to retain only those that were present on the microRNA microarrays used for both the training and validation sets, reducing the total number of probes examined to 403 miRNA probes. A panel of five microRNAs (hsa-miR-103, hsa-miR-340, hsa-miR-136, HS_{—}168, HS_{—}111) was identified correlated with prostate cancer recurrence.

| Probe Name | Coefficient |
|---|---|
| hsa-miR-103 | 0.270345 |
| hsa-miR-340 | 0.075671 |
| hsa-miR-136 | −0.09586 |
| HS_168 | −0.06271 |
| HS_111 | −0.00129 |

Kaplan-Meier analysis and the log-rank test determined that this panel could significantly discriminate patients with and without recurrence in the training set (p=1.63E-05). However, in the independent validation set, this panel was borderline significant in its ability to discriminate patients with and without recurrence (p=0.056).

An additional analysis was performed using combined data from both the 1536 protein-coding and 403 miRNA DASL probes. Combined analysis of both biomarker panels identified seven protein-coding and one miRNA gene (XPO1, hsa-miR-103, PTGDS, SOX9, RELA, EPB49, EDNRA, FOXO1A), and this combined panel was also significant in both the training set (p=1.41E-07) and the validation set (p=0.009).

| | | | | | Co- |
|---|---|---|---|---|---|
| | | | Test | | efficient |
| Probe Name | | | statistics | P-values | Estimate |
| GI_53759152-S | 732 | XPO1 | 16.33069 | 5.32E−05 | 0.190254 |
| hsa-miR-103 | hsa- | hsa- | 12.6722 | 0.000371 | 0.146229 |
| | miR- | miR-103 | | | |
| | 103 | | | | |
| GI_38505192-S | 2370 | PTGDS | 10.3771 | 0.001276 | −0.09324 |
| GI_37704387-S | 1730 | SOX9 | 10.2512 | 0.001366 | −0.03452 |
| GI_46430498-S | 2021 | RELA | 9.511572 | 0.002042 | −0.06569 |
| GI_4503580-S | 3030 | EPB49 | 9.280651 | 0.002316 | −0.09152 |
| GI_4503464-S | 3923 | EDNRA | 7.270868 | 0.007008 | 0.074626 |
| GI_9257221-S | 5330 | FOXO1A | 7.057994 | 0.007891 | −0.00292 |

Next we applied the three biomarker panels to the subset of cases in the training (n=46) and validation sets (n=18) that had a Gleason score of seven. Of the three panels, only the mRNA panel was significant (p=0.00927) at discriminating Gleason score seven cases in both the training and validation sets (see below).

| | Predictive p-value (Logrank Test) | | |
|---|---|---|---|
| | | | combined |
| | 8 mRNA | 5 miRNA | mRNA/miRNA |
| Training Set | panel | panel | panel |
| All Cases (n = 73) | 7.19E−07 | 1.63E−05 | 1.41E−07 |
| Gleason 7 Cases (n = 46) | 2.13E−05 | 0.004 | 0.000243 |
| Validation Set | | | |
| All Cases (n = 40) | 0.000153 | 0.056 | 0.009 |
| Gleason 7 Cases (n = 18) | 0.00927 | 0.69 | 0.164 |

Hierarchical clustering of the patient samples using this set of eight genes performed well in separating Gleason seven patients with and without recurrence. While the trend in the combined panel of mRNA and miRNA was towards significance (p=0.164) for the validation set, and could possibly achieve significance with a larger sample set, it did not perform as well as the mRNA panel alone.

### Example 4

Panel of ten protein-coding genes and two miRNA genes (RAD23B, FBP1, TNFRSF1A, CCNG2, NOTCH3, ETV1, BID, SIM2, ANXA1, miR-519d, and miR-647) were identified that could be used to separate patients with and without biochemical recurrence (p<0.001), as well as for the subset of 42 Gleason score 7 patients (p<0.001). an independent validation analysis on 40 samples was performed and it was found that the biomarker panel was also significant at prediction of recurrence for all cases (p=0.013) and for a subset of 19 Gleason score 7 cases (p=0.010), both of which were adjusted for relevant clinical information including T-stage, PSA and Gleason score. Importantly, these biomarkers could significantly predict clinical recurrence for Gleason 7 patients. These biomarkers may increase the accuracy of prognostication following radical prostatectomy using formalin-fixed specimens.

### Patient Samples

In the initial training set, 70 cases were used (29 with biochemical recurrence and 41 controls), 45 patients from Sunnybrook Health Science Center (Toronto, ON), and 25 patients from Emory University. The 45 cases of paraffin-embedded tissue samples from Toronto were drawn from men who underwent radical prostatectomy as the sole treatment for clinically localized prostate cancer (PCa) between 1998 and 2006. The clinical data includes multiple clinicopathologic variables such as prostate specific antigen (PSA) levels, histologic grade (Gleason score), tumor stage (pathologic stage category for example; organ confined, pT2; or with extra-prostatic extension, pT3a; or with seminal vesicle invasion, pT3b), and biochemical recurrence rates. For the cases from Emory University, both the training set (25 cases) and validation set (40 cases)

FFPE samples were also selected from a screen of over a thousand patients through an IRB-approved retrospective study at Emory University of men who had undergone radical prostatectomy. Those who were included met specific inclusion criteria, had available tissue specimens, documented long term follow-up and consented to participate or were included by IRB waiver. The cases were assigned prostate ID numbers to protect their identities. These patients did not receive neo-adjuvant or concomitant hormonal therapy. Their demographic, treatment and long-term clinical outcome data have been collected and recorded in an electronic database. Clinical data recorded include PSA measurements, radiological studies and findings, clinical findings, tissue biopsies and additional therapies that the subjects may have received.

### RNA Preparation

Tissue cores (1 mm) were used for RNA preparation rather than sections because of the heterogeneity of samples and the opportunity for obtaining cores with very high percentage tumor content. H&E stained slides were reviewed by a board certified urologic pathologist (AOO) to identify regions of cancer to select corresponding areas for cutting of cores from paraffin blocks. Total RNA was prepared at the Emory Biomarker Service Center from FFPE cores using the Ambion Recoverall MagMax methodology in 96-well format on a MagMax 96 Liquid Handler Robot (Life Technologies, Carlsbad, Calif.). FFPE RNA was quantitated by nanodrop spectrophotometry, and tested for RNA integrity and quality by Taqman analysis of the RPL13a ribosomal protein on a HT7900 real-time PCR instrument (Applied Biosystems, Foster City, Calif.). Samples with sufficient yield (>500 ng), A260/A280 ratio >1.8 and RPL13a CT values less than 30 cycles were used for miRNA and DASL profiling.

### Custom Prostate Cancer DASL Assay Pool (DAP)

The DASL assay enables quantitation of expression using RNA isolated from archived FFPE tumor tissue samples in a high throughput format. Data from multiple publicly available gene expression datasets, along with genes involved in prostate cancer progression based on state-of-the-art understanding of the disease, were distilled to develop a highly predictive set of 522 genes for use in the DASL assay. Due to specific probe design considerations, this panel had three probes for 497 genes, two probes for 20 genes, and a single probe for five genes, two of which were specific to TMPRSS2-ERG and TMPRSS2-ETV1 fusions transcripts. The unique combination of genes was optimized for performance in the DASL assay using stringent criteria that predicts performance of the primer sets. The panel includes genes found to be correlated with Gleason score. It also includes prognostic markers, and genes associated with metastasis. In addition, a number of genes known from other studies to be critical in prostate cancer such as NKX3.1, PTEN, and the Androgen Receptor are all included in the panel. Other genes that play important roles in the Wnt, Hedgehog, TGFβ, Notch, MAPK and PI3K pathways are also present in this gene set. Finally, primer sets that detect chromosomal translocations in ERG 9, ETV1 15, and ETV4 16 are also included in this panel. The optimal oligonucleotide sequence for each gene probe was determined using an oligonucleotide scoring algorithm. The oligonucleotide pool or DASL Assay Pool (DAP) was synthesized by Illumina for use with the 96-well Universal Array Matrix (UAM).

### Data Analysis

DASL fluorescent intensities were interpreted in GenomeStudio, quantile normalized, and exported for meta-analysis. Average signal intensity, genes detected (p-value=0.01), background, and noise (standard deviation of background) were analyzed for trends by plate, row, and column. The two endpoints of interest were postoperative biochemical recurrence, defined as two detectable PSA readings (>0.2 ng/ml), and clinical recurrence, defined as evidence of local or metastatic disease. The primary outcome of interest was time to biochemical recurrence following surgery. A local recurrence was defined as recurrence of cancer in the prostatic bed that was detected by either a palpable nodule on digital rectal examination (DRE) and subsequently verified by a positive biopsy, and/or a positive imaging study (prostascint or CT scan) accompanied by a detectable postoperative PSA result and lack of evidence for metastases. Also, patients whose PSA level decreased following adjuvant pelvic radiation therapy for elevated postoperative PSA were considered as local recurrence cases. A recurrence with metastases was defined as a positive imaging study indicating presence of a tumor outside of the prostatic bed.

To identify important probes and build and evaluate prediction models for prostate cancer biochemical recurrence, the following strategy was adopted. In the training step, the prediction model was built based on the time to biochemical recurrence. Specifically, we first fit a univariate Cox proportional hazard (PH) model for each individual probe using the training data set, and a set of important mRNA and miRNA probes were then preselected based on a false discovery rate (FDR) threshold of 0.30. Next, to identify the optimal prediction score based on the preselected probes, we fit a lasso Cox PH model using the training data set, where the tuning parameter for lasso was selected using a leave-one-out cross-validation technique. See Goeman, Biom J 2010, 52:70-84. The lasso Cox PH model was fitted first using the set of preselected mRNA probes only and then using the complete set of preselected mRNA and miRNA probes resulting in an optimal mRNA panel and an optimal combined mRNA/miRNA panel, respectively. Based on each biomarker panel, a final prediction model for recurrence was built to also incorporate relevant clinical biomarkers, namely, T-stage, PSA and Gleason score, through fitting Cox PH models.

To evaluate and validate the final prediction models obtained from the training phase, 79 samples from 40 patients were used and replicate samples from the same patient were again averaged to generate a single average signal for each patient. Each prediction model from the training phase was used to generate a predictive score for each subject in the validation data set, and subjects were subsequently divided into high and low scoring groups using the median predictive score. Kaplan Meier analysis was performed to compare the time to biochemical recurrence, between high (poor score) and low (good score) risk groups, and the statistical significance was determined using the log-rank test. Similarly, the final model that included both mRNA and miRNA probes for predicting time to clinical recurrence in both training and validation data sets was evaluated. The available-case approach was adopted in our analyses and the sample sizes used in each step of building and evaluating prediction models may be less than the total sample size.

### Custom Prostate DASL Profiling

DASL expression profiling with a custom-designed prostate cancer panel (see Materials and Methods section) and the Illumina DASL microRNA (miRNA) panel were performed on 70 prostatectomy patient samples to identify biomarkers predictive of recurrence. An independent validation profiling experiment was performed on 40 additional samples. MicroRNA probes were filtered to retain only those that were present on the miRNA microarrays used for both the training and validation sets, reducing the total number of probes examined to 403 microRNA probes. The training set included 29 cases with observed biochemical PSA recurrence (median time to recurrence =19 months), and 41 cases censored, i.e., without observed recurrence during the follow-up (median follow-up time=83.0 months).

### Integrated DASL Biomarker Analysis

After fitting a univariate Cox proportional hazard (PH) model for each individual probe using the training data, a set of 27 important probes were preselected based on an FDR threshold of 0.30. Next, to identify the optimal prediction score based on the preselected probes, a lasso Cox proportional hazard (PH) model was first fit using the set of 25 preselected mRNA probes only, resulting in a panel of nine protein-coding genes shown in the Table below (RAD23B, FBP1, TNFRSF1A, NOTCH3, ETV1, BID, SIM2, ANXA1, and BCL2).

| Symbol | Description | Coefficient |
|---|---|---|
| RAD23B | RAD23 homolog B | 0.152155 |
| FBP1 | Fructose-1,6-bisphosphatase 1 | 0.310566 |
| TNFRSF1A | Tumor Necrosis Factor Receptor | −0.56059 |
| | Superfamily, Member 1A | |
| NOTCH3 | Notch homolog 3 | 0.426284 |
| ETV1 | Ets Variant Gene 1 (ETV1) | 0.157241 |
| BID | BH3 Interacting Domain Death | 0.247507 |
| | Agonist (BID) | |
| SIM2 | Single-Minded Homolog 2 | 0.042942 |
| ANXA1 | Annexin A1 | −0.18514 |
| BCL2 | B-cell CLL/lymphoma 2 | 0.028339 |

A final prediction model was then built to include the predictive score based on this panel of nine mRNA biomarkers as well as the relevant clinical biomarkers including T-stage, PSA and Gleason score, which could be used to predict recurrence following radical prostatectomy. Kaplan-Meier analysis (FIG. 1A) demonstrated that these probes could significantly discriminate patients with and without recurrence by the log rank test (p<0.001). The final predictive model developed on the training set was applied to the validation set, a separate, independent DASL profiling experiment performed on a different day. Kaplan-Meier analysis (FIG. 1B) on this validation set determined that the model could discriminate patients with and without recurrence (p=0.010).

Subsequently, the above training procedure was repeated using the complete set of 27 preselected mRNA and miRNA probes, and an optimal panel of ten mRNAs and two microRNAs (additional oligonucleotides below) was identified and built as a prediction model for prostate cancer biochemical recurrence, which again included relevant clinical biomarkers. Kaplan-Meier analysis and the log-rank test determined that this panel could significantly discriminate patients with and without recurrence both in the training set (p<0.001, FIG. 1C) and in the validation set (p=0.013, FIG. 1D).

FBP1
(SEQ ID NO: 178)
5′-ACTTCGTCAGTAACGGACTGGCATTGCTGGTTCTACCAAC-3′
(SEQ ID NO: 179)
5′-GAGTCGAGGTCATATCGTTGGCATTGCTGGTTCTACCAAC-3′
(SEQ ID NO: 180)
5′-TGACAGGTGATCAAGTTAAGAAGTCGAGCGTTCGGAGCACTTAATCG
TCTGCCTATAGTGAGTC-3′
TNFRSF1A
(SEQ ID NO: 181)
5′-ACTTCGTCAGTAACGGACTCCCCAAGGAAAATATATCCACCC-3′
(SEQ ID NO: 182)
5′-GAGTCGAGGTCATATCGTTCCCCAAGGAAAATATATCCACCC-3′
(SEQ ID NO: 183)
5′-CAAAATAATTCGATTTGCTGTACAGTAGCCCAGGTAGCGGAGCTTGT
CTGCCTATAGTGAGTC-3′
NOTCH3
(SEQ ID NO: 184)
5′-ACTTCGTCAGTAACGGACGTTCACAGGAACCTATTGCGAGGT-3′
(SEQ ID NO: 185)
5′-GAGTCGAGGTCATATCGTGTTCACAGGAACCTATTGCGAGGT-3′
(SEQ ID NO: 186)
5′-GACATTGACGAGTGTCAGAGTAGCTGACTCTTGTAGTATTGCGCGAA
GTCTGCCTATAGTGAGTC-3′
ETV1
(SEQ ID NO: 187)
5′-ACTTCGTCAGTAACGGACTATGTTTGAAAAGGGCCCCAGG-3′
(SEQ ID NO: 188)
5′-GAGTCGAGGTCATATCGTTATGTTTGAAAAGGGCCCCAGG-3′
(SEQ ID NO: 189)
5′-AGTTTTATGATGACACCTGTGTTTACGGATGGCAACAAGTACGGATT
GTCTGCCTATAGTGAGTC-3′
BID
(SEQ ID NO: 190)
5′-ACTTCGTCAGTAACGGACGTTCCAGCCTCAGGGATGAGTG-3′
(SEQ ID NO: 191)
5′-GAGTCGAGGTCATATCGTGTTCCAGCCTCAGGGATGAGTG-3′
(SEQ ID NO: 192)
5′-ATCACAAACCTACTGGTGTTTGGCGCTAGGTTAATAAGCGGATGCGT
CTGCCTATAGTGAGTC-3′
ANXA1
(SEQ ID NO: 193)
5′-ACTTCGTCAGTAACGGACGATCAGAATTCCTCAAGCAGGCC-3′
(SEQ ID NO: 194)
5′-GAGTCGAGGTCATATCGTGATCAGAATTCCTCAAGCAGGCC-3′
(SEQ ID NO: 195)
5′-GGTTTATTGAAAATGAAGAGCAAGGGTTCTATGTTTGGACGCCATGG
TCTGCCTATAGTGAGTC-3′
BCL2
(SEQ ID NO: 196)
5′-ACTTCGTCAGTAACGGACCGTGCCTCATGAAATAAAGATCCG-3′
(SEQ ID NO: 197)
5′-GAGTCGAGGTCATATCGTCGTGCCTCATGAAATAAAGATCCG-3′
(SEQ ID NO: 198)
5′-AAGGAATTGGAATAAAAATTTCCGGATGACGACCGAATACCGTTGGT
CTGCCTATAGTGAGTC-3′
CCNG2
(SEQ ID NO: 199)
5′-ACTTCGTCAGTAACGGACGCCACTCATGATGTGATCCGGATT-3′
(SEQ ID NO: 200)
5′-GAGTCGAGGTCATATCGTGCCACTCATGATGTGATCCGGATT-3′
(SEQ ID NO: 201)
5′-GTCAGTGTAAATGTACTGCTTCTGGTGCTCTGAGACGGCAAAGATTC
GTCTGCCTATAGTGAGTC-3′
hsa-miR-647
ProbeSeq
(SEQ ID NO: 202)
5′-GTGGCTGCACTCACTTC-3′
TargetMatureSeqs
(SEQ ID NO: 203)
5′-GTGGCTGCACTCACTTCCTTC-3′
Oligo
(SEQ ID NO: 204)
5′-ACTTCGTCAGTAACGGACTTGAGCGGACCCAGA
TGTACCGGTGGCTGCACTCACTTC-3′
hsa-miR-519d
ProbeSeq
(SEQ ID NO: 205)
5′-AGTGCCTCCCTTTAGAGTG-3′
TargetMatureSeqs
(SEQ ID NO: 206)
5′-CAAAGTGCCTCCCTTTAGAGTG-3′
Oligo
(SEQ ID NO: 207)
5′-ACTTCGTCAGTAACGGACCAGAGTGTCCCCGT
GGCGATACAGTGCCTCCCTTTAGAGTG-3′
RAD23B
(SEQ ID NO: 13)
5′-ACTTCGTCAGTAACGGACAATCCTTCCTTGCTTCCAGCG-3′
(SEQ ID NO: 14)
5′-GAGTCGAGGTCATATCGTAATCCTTCCTTGCTTCCAGCG-3′
(SEQ ID NO: 208)
5′-TACTACAGCAGATAGGTCGAGAGTAGGGTTCGGGTTCAGACACCGGT
CTGCCTATAGTGAGTC-3′

Prediction of Cases with a Gleason Score 7

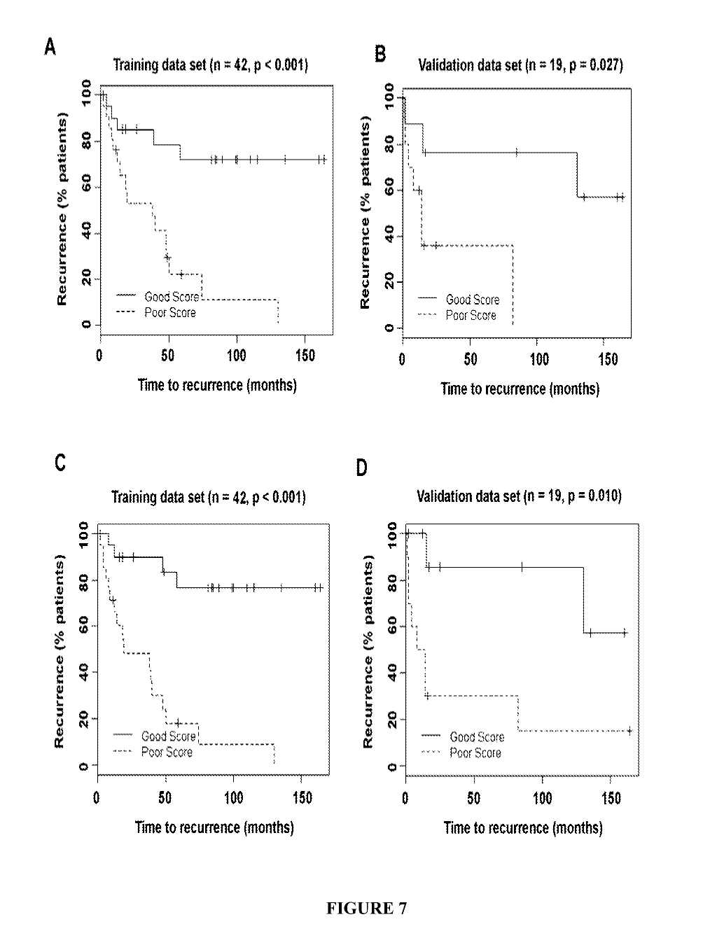

Prediction of recurrence for patients with a Gleason score 7 is particularly difficult. In order to address this issue, we applied the biomarker panels to the subset of cases in the training and validation sets that had a Gleason score 7. The prediction model based on the nine-mRNA panel was significant at discriminating biochemical recurrence in Gleason score 7 cases in both the training set (p<0.001, FIG. 7A) and the validation set (p=0.027, FIG. 7B). For the prediction model based on the combined panel of ten mRNAs and two miRNAs in the tables below, the predictive value was again significant for both the training set (p=<0.001, FIG. 7C) and the validation set (p=0.010, FIG. 7D).

| Symbol | Description | Coefficient |
|---|---|---|
| RAD23B | RAD23 homolog B | 0.070324 |
| FBP1 | Fructose-1,6-bisphosphatase 1 | 0.251286 |
| TNFRSF1A | Tumor necrosis factor receptor | −0.58801 |
| | superfamily, member 1A | |
| CCNG2 | Cyclin G2 | 0.008039 |
| hsa-miR- | hsa-miR-647 | −0.31794 |
| 647 | | |
| LETMD1 | LETM1 domain containing 1 | 0.063197 |
| NOTCH3 | Notch homolog 3 | 0.366933 |
| ETV1 | ETS variant gene 1 (ETV1) | 0.179233 |
| hsa-miR- | hsa-miR-519d | 0.550635 |
| 519d | | |
| BID | BH3 interacting domain death agonist (BID) | 0.128237 |
| SIM2 | Single-minded homolog 2 | 0.124271 |
| ANXA1 | Annexin A1 | −0.14319 |

| | | Combined |
|---|---|---|
| | | mRNA/miRNA |
| | mRNA panel | panel |
| Training Set | | |
| All Cases (n = 61) | <0.001 | <0.001 |
| Gleason 7 Cases (n = 42) | <0.001 | <0.001 |
| Validation Set | | |
| All Cases (n = 35) | 0.01 | 0.013 |
| Gleason 7 Cases (n = 19) | 0.027 | 0.01 |

### Analysis of Clinical Recurrence

Although most patients who have clinical recurrence following prostatectomy also have biochemical recurrence, there is a significant population of patients with biochemical recurrence who do not have clinically significant recurrences observed during their follow-ups. To evaluate our biomarker panel of biochemical recurrence for predicting the clinical recurrence, the prediction model was tested based on the combined mRNA/miRNA panel in the same training and validation samples using their clinical recurrence outcome data. Unfortunately, clinical recurrence data was lacking on some of the samples, and the total number of samples used in the training set was reduced. In the training data, the combined mRNA/miRNA panel was highly significant for predicting recurrence in all patients (p=0.002) as well as in the subset of patients with a Gleason score 7 (p=0.004); in the validation data, it was also significant for predicting recurrence in patients with a Gleason score 7 (p=0.023) and trended towards significance in all patients (p=0.078).

| | Combined mRNA/ |
|---|---|
| | miRNA panel |
| Training Set | |
| All Cases (n = 56) | 0.002 |
| Gleason 7 Cases (n = 37) | 0.004 |
| Validation Set | |
| All Cases (n = 35) | 0.078 |
| Gleason 7 Cases (n = 19) | 0.023 |

An analysis was also performed to construct a predictive set of biomarkers based on the clinical recurrence data instead of biochemical recurrence. Only three probes passed the initial preselection step for the univariate Cox PH modeling, all corresponding to the ETV1 gene. Furthermore, the prediction model built on clinical recurrence did not perform as well as the model built on biochemical recurrence, which is likely due to the considerably less number of clinical recurrences in the training set as well as the smaller total sample size.

### Discussion

The DASL assay has been used to identify a 16-gene set that correlates with prostate cancer relapse. Bibikova et al., Genomics 2007, 89:666-672. Overlap between our panel of ten mRNA and two miRNA biomarkers described here and the previously described 16-gene panel was limited to FBP1 even though ten of the genes in the 16-gene panel reported were included in our 522 custom prostate DASL panel. When the performance of the probes corresponding to those ten mRNAs was analyzed in our dataset, they were not able to significantly discriminate patients at higher and lower risk of recurrence. The gene signature selection and prediction model building were performed in separate steps and the signature selection was based on the correlation between the gene expression and Gleason score rather than between the gene expression and time to biochemical recurrence; our analytic approach overcomes these limitations. Specifically, our approach of building (training) prediction models takes advantage of recent advancement in regularized regression models for survival outcomes; regularized regression models can achieve simultaneous feature selection and model estimation and avoid model overfitting leading to better prediction performance.

Two other studies have employed DASL profiling to prostate cancer, but not detected any signature that improved upon clinical models in validation sets. Sboner et al., BMC Med Genomics 2010, 3:8 and Nakagawa et al., PLoS ONE, 2008, 3:e2318. While these studies used large cohorts with long-term follow-up, they did not include probes corresponding to microRNA genes. Moreover, these earlier studies suggested that tumor heterogeneity may play an important role in confounding signature identification. For our study of prostatectomy specimens, the most prominent tumor lesion were identified, and used a tissue core sample from that region to minimize stromal contributions and tumor heterogeneity.

In our twelve-gene predictive biomarker panel, nine of the genes are positively associated with recurrence, and three are negatively associated with recurrence. The nine genes positively associated with recurrence included miR-519d, Notch homolog 3 (Notch3), Fructose-1,6-bisphosphatase 1 (FBP1), ETS variant gene 1 (ETV1), BH3 interacting domain death agonist (BID), Single-Minded homolog 2 (SIM2), RAD23 homolog B (RAD23B), LETM1 domain containing 1 (LETMD1), and Cyclin G2 (CCNG2). Little is known about miR-519d other than it may be associated with obesity. Martinelli et al., miR-519d Overexpression Is Associated With Human Obesity, Obesity (Silver Spring) 2010. NOTCH3 is one of four Notch family receptors in humans, and Notch signaling has been shown to be important for prostate cancer cell growth, migration, and invasion as well as normal prostate development. FBP1 is expressed in the prostate and is involved in gluconeogenesis. The identification of this metabolic enzyme as a biomarker of recurrence is initially surprising. FBP1 was overexpressed in independent microarray analyses of prostate cancers. ETV1 is one of the recurrent translocations found in prostate cancers, and has been used in clinical models of recurrence following prostatectomy. Cheville et al., J Clin Oncol 2008, 26:3930-3936. BID is a pro-apoptotic protein that binds to BCL2 and potentiates apoptotic responses upon cleavage in response to tumor necrosis factor alpha (TNFα) and other death receptors. SIM2 was identified as a potential biomarker of prostate cancer. Halvorsen et al., Clin Cancer Res 2007, 13:892-897. SIM2 functions as a transcription factor that represses the proapoptotic gene BNIP3. RAD23B plays a role in DNA damage recognition and nucleotide excision repair, as well as inhibiting MDM2 mediated degradation of the p53 tumor suppressor. LETMD1 (also known as HCCR) is an oncogene that is induced by Wnt and PI3K/AKT signaling, inhibits p53 function, and is a biomarker for hepatocellular and breast cancers. Cyclin G2 is an atypical cyclin that is induced by DNA damage in a p53-independent manner, as well as by PI3K/AKT/FOXO signals, and induces p53-dependent cell cycle arrest.

The three genes in the predictive biomarker panel negatively associated with recurrence were miR-647, the TNFα receptor (TNFRSF1A), and annexin A1 (ANXA1). While little is known about miR-647, TNFRSF1A (also known as TNFR1) mediates pro-apoptotic responses to TNFα ligand Annexin A1 expression is reduced in early onset prostate cancer and high-grade prostatic intraepithelial neoplasia. ANXA1 plays roles in vesicle trafficking and reduced ANXA1 promotes EMT and metastasis, and upregulates autocrine IL-6 signaling.

1. A method of predicting the recurrence, progression, and metastatic potential of a cancer in a subject, the method comprising detecting in a sample from the subject one or more biomarkers selected from the group consisting of CSPG2, WNT10B, E2F3, CDKN2A, TYMS, TGFB3, ALOX12, CD44, LAF4, CTNNA1, XPO1, PTGDS, SOX9, RELA, EPB49, SIM2, EDNRA, RAD23B, FBP1, TNFRSF1A, CCNG2, LETMD1, NOTCH3, ETV1, BID, SIM2, ANXA1, BCL2, miR-519d, miR-647, FOXO1A, SOX9, CLNS1A, PTGDS, XPO1, LETMD1, RAD23B, ABCC3, APC, CHES1, EDNRA, FRZB, HSPG2, TMPRSS2_ETV1 FUSION, CSPG2, WNT10B, E2F3, CDKN2A, TYMS, miR-103, miR-339, miR-183, miR-182, miR-136, and/or miR-221, wherein an increase or decrease in one or more of the biomarkers as compared to a standard indicating a recurrent, progressive, or metastatic cancer.

2. The method of claim 1, wherein the sample comprises prostate tumor tissue.

3. The method of claim 1, wherein the cancer comprises a TMPRSS2-ERG fusion-positive prostate cancer.

4. The method of claim 1, wherein the detecting step comprises detecting mRNA and miRNA expression level patterns of the biomarkers.

5. The method of claim 4, wherein the RNA detection comprises reverse-transcription polymerase chain reaction (RT-PCR) assay; quantitative real-time-PCR (qRT-PCR); Northern analysis; microarray analysis; or cDNA-mediated annealing, selection, extension, and ligation (DASL) assay.

6. The method of claim 1, further comprising detecting in a sample from the subject two, three, four, five, six, seven, eight or more biomarkers selected from the group consisting of CSPG2, WNT10B, E2F3, CDKN2A, TYMS, TGFB3, ALOX12, CD44, and LAF4.

7. The method of claim 1, wherein the detected biomarkers comprise two, three, four, five, six, seven, eight, nine, ten, eleven, twelve, thirteen, fourteen, fifteen, sixteen, seventeen, eighteen, nineteen, twenty or more biomarkers selected from the group consisting of FOXO1A, SOX9, CLNS1A, PTGDS, XPO1, LETMD1, RAD23B, ABCC3, APC, CHES1, EDNRA, FRZB, HSPG2, TMPRSS2_ETV1 FUSION, miR-103, miR-339, miR-183, miR-182, miR-136, and miR-221.

8. The method of claim 1, wherein the detected biomarkers are selected from the group consisting of miR-519d and/or miR-647 and two, three, four, five, six, seven, eight, nine or more markers selected from the group consisting of RAD23B, FBP1, TNFRSF1A, CCNG2, LETMD1, NOTCH3, ETV1, BID, SIM2, and ANXA1.

9. A method of treating a subject with cancer comprising modifying a treatment regimen of the subject based on the results of the method of claim 1.

10. The method of claim 9, wherein the treatment regimen is modified to be aggressive based on an increase in one or more biomarkers selected from the group consisting of CSPG2, WNT10B, E2F3, CDKN2A, and TYMS as compared to a standard, and a decrease in one or more biomarkers selected from the group consisting of TGFB3, ALOX12, CD44, and LAF4 as compared to a standard.

11. The method of claim 9, wherein the treatment regimen is further modified to be aggressive based on an increase in one or more biomarkers selected from the group consisting of CLNS1A, XPO1, LETMD1, RAD23B, TMPRSS2_ETV1 FUSION, ABCC3, SPC, CHES1, FRZB, HSPG2, miR-103, miR-339, miR-183, and miR-182 as compared to a standard, and a decrease in one or more biomarkers selected from the group consisting of FOXO1A, SOX9, PTGDS, EDNRA, miR-136, and miR-221 as compared to a standard.

12. The method of claim 9, wherein the treatment regimen is further modified to be aggressive based on an increased expression of RAD23B, FBP1, CCNG2, LETMD1, NOTCH3, ETV1, BID, SIM2, miR-519d and the decreased expression of TNFRSF1A, miR-647, and ANXA1.

13. A method of predicting the recurrence, progression, and metastatic potential of a prostate cancer in a subject, the method comprising analyzing a sample from the subject for an aberrant expression pattern of four or more biomarkers wherein at least one of the biomarkers is a microRNA selected from miR-519d, miR-647, miR-103, miR-339, miR-183, and miR-182 miR-136, and/or miR-221.

14. A method of predicting the recurrence, progression, and metastatic potential of a cancer in a subject, the method comprising detecting in a sample from the subject an increase in miR-519d.

15. A method of predicting the recurrence, progression, and metastatic potential of a cancer in a subject, the method comprising detecting in a sample from the subject a decrease in miR-647.


SEQUENCE LISTING (208 sequences)

SEQ ID NO: 1 - DNA, 42 bp, Artificial Sequence
  FOXO1A
acttcgtcagtaacggacgtcctaggagaagagctgcatcca

SEQ ID NO: 2 - DNA, 42 bp, Artificial Sequence
  SOX9
gagtcgaggtcatatcgtgtcctaggagaagagctgcatcca

SEQ ID NO: 3 - DNA, 39 bp, Artificial Sequence
  SOX9
acttcgtcagtaacggacgctcctacccgcccatcaccc

SEQ ID NO: 4 - DNA, 39 bp, Artificial Sequence
  SOX9
gagtcgaggtcatatcgtgctcctacccgcccatcaccc

SEQ ID NO: 5 - DNA, 41 bp, Artificial Sequence
  SOX9
acttcgtcagtaacggacggagagaacttggtgcctcttcc

SEQ ID NO: 6 - DNA, 41 bp, Artificial Sequence
  CLNS1A
gagtcgaggtcatatcgtggagagaacttggtgcctcttcc

SEQ ID NO: 7 - DNA, 37 bp, Artificial Sequence
  CLNS1A
acttcgtcagtaacggacgcgaacccagacccccagg

SEQ ID NO: 8 - DNA, 37 bp, Artificial Sequence
  PTGDS
gagtcgaggtcatatcgtgcgaacccagacccccagg

SEQ ID NO: 9 - DNA, 41 bp, Artificial Sequence
  PTGDS
acttcgtcagtaacggacgccagcaaagaatggctcaagaa

SEQ ID NO: 10 - DNA, 41 bp, Artificial Sequence
  XPO1
gagtcgaggtcatatcgtgccagcaaagaatggctcaagaa

SEQ ID NO: 11 - DNA, 42 bp, Artificial Sequence
  XPO1
acttcgtcagtaacggacgtcacctttctccaaaggcagatg

SEQ ID NO: 12 - DNA, 42 bp, Artificial Sequence
  LETMD
gagtcgaggtcatatcgtgtcacctttctccaaaggcagatg

SEQ ID NO: 13 - DNA, 39 bp, Artificial Sequence
  RAD23B
acttcgtcagtaacggacaatccttccttgcttccagcg

SEQ ID NO: 14 - DNA, 39 bp, Artificial Sequence
  RAD23B
gagtcgaggtcatatcgtaatccttccttgcttccagcg

SEQ ID NO: 15 - DNA, 38 bp, Artificial Sequence
  TMPRSS
acttcgtcagtaacggacagcgcggcactcaggtacct

SEQ ID NO: 16 - DNA, 38 bp, Artificial Sequence; identical to SEQ ID NO: 15
  2_ETV1 FUSION

SEQ ID NO: 17 - DNA, 40 bp, Artificial Sequence
  ABCC3
acttcgtcagtaacggacatgttcctgtgctccatgatgc

SEQ ID NO: 18 - DNA, 40 bp, Artificial Sequence
  ABCC3
gagtcgaggtcatatcgtatgttcctgtgctccatgatgc

SEQ ID NO: 19 - DNA, 63 bp, Artificial Sequence
  ABCC3
gtcgctgatcttacaacactattacatgcctattgacgtgaggcggtctgcctatagtgagtc

SEQ ID NO: 20 - DNA, 41 bp, Artificial Sequence
  APC
acttcgtcagtaacggacgtccctggagtaaaactgcggtc

SEQ ID NO: 21 - DNA, 41 bp, Artificial Sequence
  APC
gagtcgaggtcatatcgtgtccctggagtaaaactgcggtc

SEQ ID NO: 22 - DNA, 63 bp, Artificial Sequence
  APC
aaaatgtccctccgttcttatctagatcgcaaaagtgtctcggaagtctgcctatagtgagtc

SEQ ID NO: 23 - DNA, 39 bp, Artificial Sequence
  CHES1
acttcgtcagtaacggacgggtttctccaaggcccttca

SEQ ID NO: 24 - DNA, 39 bp, Artificial Sequence
  CHESI
gagtcgaggtcatatcgtgggtttctccaaggcccttca

SEQ ID NO: 25 - DNA, 62 bp, Artificial Sequence
  CHES1
gaagacgatgacctcgacttcatacgcgaattgatagaagctcggtctgcctatagtgagtc

SEQ ID NO: 26 - DNA, 41 bp, Artificial Sequence
  ENDRA
acttcgtcagtaacggactgcaactctgctcaggatcattt

SEQ ID NO: 27 - DNA, 41 bp, Artificial Sequence
  EDNRA
gagtcgaggtcatatcgttgcaactctgctcaggatcattt

SEQ ID NO: 28 - DNA, 62 bp, Artificial Sequence
  EDNRA
ccagaacaaatgtatgaggaattcactcaaggccgttagctgtggtctgcctatagtgagtc

SEQ ID NO: 29 - DNA, 43 bp, Artificial Sequence
  FRZB
acttcgtcagtaacggacggaagcttcgtcatcttggactcag

SEQ ID NO: 30 - DNA, 43 bp, Artificial Sequence
  FRZB
gagtcgaggtcatatcgtggaagcttcgtcatcttggactcag

SEQ ID NO: 31 - DNA, 66 bp, Artificial Sequence
  FRZB
aaaagtgattctagcaatagtgattttactgcgctcctaattggcaccgtctgcctatagtgagtc

SEQ ID NO: 32 - DNA, 38 bp, Artificial Sequence
  HSPG2
acttcgtcagtaacggacccaaatgcgctggacacatt

SEQ ID NO: 33 - DNA, 38 bp, Artificial Sequence
  HSPG2
gagtcgaggtcatatcgtccaaatgcgctggacacatt

SEQ ID NO: 34 - DNA, 62 bp, Artificial Sequence
  HSPG2
gtacctttctgatgatgaggacggaacagcttacgactttgcgggtctgcctatagtgagtc

SEQ ID NO: 35 - DNA, 47 bp, Artificial Sequence
  FOXO1A
tcctaggagaagagctgcatccatggacaacaacagtaaatttgcta

SEQ ID NO: 36 - DNA, 43 bp, Artificial Sequence
  SOX9
ctcctacccgcccatcacccgctcacagtacgactacaccgac

SEQ ID NO: 37 - DNA, 47 bp, Artificial Sequence
  CLNS1A
ggagagaacttggtgcctcttccactctggagtgaagttaatgaaag

SEQ ID NO: 38 - DNA, 42 bp, Artificial Sequence
  PTGDS
cgaacccagacccccagggctgagttaaaggagaaattcacc

SEQ ID NO: 39 - DNA, 46 bp, Artificial Sequence
  XPO1
ccagcaaagaatggctcaagaagtactgacacatttaaaggagcat

SEQ ID NO: 40 - DNA, 47 bp, Artificial Sequence
  LETMD1
tcacctttctccaaaggcagatgtgaagaacttgatgtcttatgtgg

SEQ ID NO: 41 - DNA, 45 bp, Artificial Sequence
  RAD23B
aatccttccttgcttccagcgttactacagcagataggtcgagag

SEQ ID NO: 42 - DNA, 43 bp, Artificial Sequence
  TMPRSS2_ETV1 FUSION
agcgcggcactcaggtacctgacaatgatgagcagtttgtacc

SEQ ID NO: 43 - DNA, 46 bp, Artificial Sequence
  ABCC3
atgttcctgtgctccatgatgcagtcgctgatcttacaacactatt

SEQ ID NO: 44 - DNA, 44 bp, Artificial Sequence
  APC
tccctggagtaaaactgcggtcaaaaatgtccctccgttcttat

SEQ ID NO: 45 - DNA, 41 bp, Artificial Sequence
  CHES1
ggtttctccaaggcccttcaggaagacgatgacctcgactt

SEQ ID NO: 46 - DNA, 46 bp, Artificial Sequence
  EDNRA
tgcaactctgctcaggatcatttaccagaacaaatgtatgaggaat

SEQ ID NO: 47 - DNA, 50 bp, Artificial Sequence
  FRZB
gaagcttcgtcatcttggactcagtaaaagtgattctagcaatagtgatt

SEQ ID NO: 48 - DNA, 43 bp, Artificial Sequence
  HSPG2
ccaaatgcgctggacacattcgtacctttctgatgatgaggac

SEQ ID NO: 49 - DNA, 41 bp, Artificial Sequence
  ALOX12
acttcgtcagtaacggacgttacgctttgcagaccgcatag

SEQ ID NO: 50 - DNA, 41 bp, Artificial Sequence
  ALOX12
gagtcgaggtcatatcgtgttacgctttgcagaccgcatag

SEQ ID NO: 51 - DNA, 41 bp, Artificial Sequence
  ALOX12
acttcgtcagtaacggacgatcgctgcagaccgtaaggatg

SEQ ID NO: 52 - DNA, 41 bp, Artificial Sequence
  ALOX12
gagtcgaggtcatatcgtgatcgctgcagaccgtaaggatg

SEQ ID NO: 53 - DNA, 41 bp, Artificial Sequence
  ALOX12
acttcgtcagtaacggacctaaggctctatttcctccccca

SEQ ID NO: 54 - DNA, 41 bp, Artificial Sequence
  ALOX12
gagtcgaggtcatatcgtctaaggctctatttcctccccca

SEQ ID NO: 55 - DNA, 40 bp, Artificial Sequence
  CD44
acttcgtcagtaacggaccacccgctatgtccagaaagga

SEQ ID NO: 56 - DNA, 40 bp, Artificial Sequence
  CD44
gagtcgaggtcatatcgtcacccgctatgtccagaaagga

SEQ ID NO: 57 - DNA, 41 bp, Artificial Sequence
  CD44
acttcgtcagtaacggacgctaatccctgggcattgctttc

SEQ ID NO: 58 - DNA, 41 bp, Artificial Sequence
  CD44
gagtcgaggtcatatcgtgctaatccctgggcattgctttc

SEQ ID NO: 59 - DNA, 41 bp, Artificial Sequence
  CD44
acttcgtcagtaacggaccagctgatgagacaaggaacctg

SEQ ID NO: 60 - DNA, 41 bp, Artificial Sequence
  CD44
gagtcgaggtcatatcgtcagctgatgagacaaggaacctg

SEQ ID NO: 61 - DNA, 43 bp, Artificial Sequence
  CDKN2A
acttcgtcagtaacggacgggaagctgtcgacttcatgacaag

SEQ ID NO: 62 - DNA, 43 bp, Artificial Sequence
  CDKN2A
gagtcgaggtcatatcgtgggaagctgtcgacttcatgacaag

SEQ ID NO: 63 - DNA, 38 bp, Artificial Sequence
  CDKN2A
acttcgtcagtaacggacgaacccaccccgctttcgta

SEQ ID NO: 64 - DNA, 38 bp, Artificial Sequence
  CDKN2A
gagtcgaggtcatatcgtgaacccaccccgctttcgta

SEQ ID NO: 65 - DNA, 42 bp, Artificial Sequence
  CDKN2A
acttcgtcagtaacggacgcgcttctgccttttcactgtgtt

SEQ ID NO: 66 - DNA, 42 bp, Artificial Sequence
  CDKN2A
gagtcgaggtcatatcgtgcgcttctgccttttcactgtgtt

SEQ ID NO: 67 - DNA, 41 bp, Artificial Sequence
  CSPG2
acttcgtcagtaacggacccacagtccaacctcaggctatc

SEQ ID NO: 68 - DNA, 41 bp, Artificial Sequence
  CSPG2
gagtcgaggtcatatcgtccacagtccaacctcaggctatc

SEQ ID NO: 69 - DNA, 40 bp, Artificial Sequence
  CSPG2
acttcgtcagtaacggacgcatggaaggaagtgctttggg

SEQ ID NO: 70 - DNA, 40 bp, Artificial Sequence
  CSPG2
gagtcgaggtcatatcgtgcatggaaggaagtgctttggg

SEQ ID NO: 71 - DNA, 40 bp, Artificial Sequence
  CSPG2
acttcgtcagtaacggactgctccagagtacaactggcgt

SEQ ID NO: 72 - DNA, 40 bp, Artificial Sequence
  CSPG2
gagtcgaggtcatatcgttgctccagagtacaactggcgt

SEQ ID NO: 73 - DNA, 41 bp, Artificial Sequence
  ESF3
acttcgtcagtaacggacgctcaggatggggtcagatggag

SEQ ID NO: 74 - DNA, 41 bp, Artificial Sequence
  E2F3
gagtcgaggtcatatcgtgctcaggatggggtcagatggag

SEQ ID NO: 75 - DNA, 41 bp, Artificial Sequence
  E2F3
acttcgtcagtaacggactaagttggaccaagggaagtcgg

SEQ ID NO: 76 - DNA, 41 bp, Artificial Sequence
  E2F3
gagtcgaggtcatatcgttaagttggaccaagggaagtcgg

SEQ ID NO: 77 - DNA, 41 bp, Artificial Sequence
  E2F3
acttcgtcagtaacggacaggtttatcagcctctgcaagga

SEQ ID NO: 78 - DNA, 41 bp, Artificial Sequence
  E2F3
gagtcgaggtcatatcgtaggtttatcagcctctgcaagga

SEQ ID NO: 79 - DNA, 41 bp, Artificial Sequence
  LAF4
acttcgtcagtaacggactcctctaacaagtggcagctgga

SEQ ID NO: 80 - DNA, 41 bp, Artificial Sequence
  LAF4
gagtcgaggtcatatcgttcctctaacaagtggcagctgga

SEQ ID NO: 81 - DNA, 40 bp, Artificial Sequence
  LAF4
acttcgtcagtaacggacgggagatcaagaagtcccaggg

SEQ ID NO: 82 - DNA, 40 bp, Artificial Sequence
  LAF4
gagtcgaggtcatatcgtgggagatcaagaagtcccaggg

SEQ ID NO: 83 - DNA, 42 bp, Artificial Sequence
  LAF4
acttcgtcagtaacggacgtctgatccaaaatgaaagccacg

SEQ ID NO: 84 - DNA, 42 bp, Artificial Sequence
  LAF4
gagtcgaggtcatatcgtgtctgatccaaaatgaaagccacg

SEQ ID NO: 85 - DNA, 41 bp, Artificial Sequence
  TGFB3
acttcgtcagtaacggacgaggggatggggatagaggaaag

SEQ ID NO: 86 - DNA, 41 bp, Artificial Sequence
  TGFB3
gagtcgaggtcatatcgtgaggggatggggatagaggaaag

SEQ ID NO: 87 - DNA, 42 bp, Artificial Sequence
  TGFB3
acttcgtcagtaacggacgcatgtcacacctttcagcccaat

SEQ ID NO: 88 - DNA, 42 bp, Artificial Sequence
  TGFB3
gagtcgaggtcatatcgtgcatgtcacacctttcagcccaat

SEQ ID NO: 89 - DNA, 42 bp, Artificial Sequence
  TGFB3
acttcgtcagtaacggaccggtggtaaagaaagtgtgggttt

SEQ ID NO: 90 - DNA, 42 bp, Artificial Sequence
  TGFB3
gagtcgaggtcatatcgtcggtggtaaagaaagtgtgggttt

SEQ ID NO: 91 - DNA, 41 bp, Artificial Sequence
  TYMS
acttcgtcagtaacggacgggtgctttcaaaggagcttgaa

SEQ ID NO: 92 - DNA, 41 bp, Artificial Sequence
  TYMS
gagtcgaggtcatatcgtgggtgctttcaaaggagcttgaa

SEQ ID NO: 93 - DNA, 40 bp, Artificial Sequence
  TYMS
acttcgtcagtaacggacttgacaccatcaaaaccaaccc

SEQ ID NO: 94 - DNA, 40 bp, Artificial Sequence
  TYMS
gagtcgaggtcatatcgtttgacaccatcaaaaccaaccc

SEQ ID NO: 95 - DNA, 42 bp, Artificial Sequence
  TYMS
acttcgtcagtaacggacagggatccacaaatgctaaagagc

SEQ ID NO: 96 - DNA, 42 bp, Artificial Sequence
  TYMS
gagtcgaggtcatatcgtagggatccacaaatgctaaagagc

SEQ ID NO: 97 - DNA, 40 bp, Artificial Sequence
  WNT10B
cttcgtcagtaacggacccacccctcttctgctccttaga

SEQ ID NO: 98 - DNA, 41 bp, Artificial Sequence
  WNT10B
gagtcgaggtcatatcgtccacccctcttctgctccttaga

SEQ ID NO: 99 - DNA, 41 bp, Artificial Sequence
  WNT10B
acttcgtcagtaacggacgctgtccaggcccttagggaagt

SEQ ID NO: 100 - DNA, 41 bp, Artificial Sequence
  WNT10B
gagtcgaggtcatatcgtgctgtccaggcccttagggaagt

SEQ ID NO: 101 - DNA, 42 bp, Artificial Sequence
  WNT10B
acttcgtcagtaacggactgctgtgtgatgagtgcaaggtta

SEQ ID NO: 102 - DNA, 42 bp, Artificial Sequence
  WNT10B
gagtcgaggtcatatcgttgctgtgtgatgagtgcaaggtta

SEQ ID NO: 103 - DNA, 63 bp, Artificial Sequence
  ALOX12
cactgtctcaactactcagctctcctgatacgcgagcctagacgtgtctgcctatagtgagtc

SEQ ID NO: 104 - DNA, 63 bp, Artificial Sequence
  ALOX12
tctacctccaaatatgagattcctgtagccctacgcgacggttgagtctgcctatagtgagtc

SEQ ID NO: 105 - DNA, 63 bp, Artificial Sequence
  ALOX12
ttaaaccccctacattagtatcctacacagcgaccgtaccatcgtgtctgcctatagtgagtc

SEQ ID NO: 106 - DNA, 63 bp, Artificial Sequence
  CD44
aatacagaacgaatcctgaagacaaagccgatcttcgcccagtctgtctgcctatagtgagtc

SEQ ID NO: 107 - DNA, 62 bp, Artificial Sequence
  CD44
actgaggttggggtgtactagtaagggtgcgacactatctcgacgtctgcctatagtgagtc

SEQ ID NO: 108 - DNA, 61 bp, Artificial Sequence
  CD44
agaatgtggacatgaagattggttctaatgggcgcaccaaaccgtctgcctatagtgagtc

SEQ ID NO: 109 - DNA, 62 bp, Artificial Sequence
  CDKN2A
attttgtgaactagggaagctcgcctggcgaataaaggtcgtacgtctgcctatagtgagtc

SEQ ID NO: 110 - DNA, 64 bp, Artificial Sequence
  CDKN2A
ttttcatttagaaaatagagcttttcgttacatccatcgcagcgacgtctgcctatagtgagtc

SEQ ID NO: 111 - DNA, 61 bp, Artificial Sequence
  CDKN2A
gagttttctggagtgagcactaattgggtctcgcagtagtggcgtctgcctatagtgagtc

SEQ ID NO: 112 - DNA, 63 bp, Artificial Sequence
  CSPG2
cagatagtttagccaccaaattaaacgatgtccgtgattgcctgggtctgcctatagtgagtc

SEQ ID NO: 113 - DNA, 63 bp, Artificial Sequence
  CSPG2
gaagtagaagatgtggacctctccaaataggccgtgtcctccgtggtctgcctatagtgagtc

SEQ ID NO: 114 - DNA, 62 bp, Artificial Sequence
  CSPG2
tctcattatgctacggattcattagggttcgggttcagacaccggtctgcctatagtgagtc

SEQ ID NO: 115 - DNA, 62 bp, Artificial Sequence
  E2F3
gacctctagggagaaagacatcacctatttggcggaggaccactgtctgcctatagtgagtc

SEQ ID NO: 116 - DNA, 62 bp, Artificial Sequence
  E2F3
gacgtaaaaaatgaagcaaaactagctggcccacgaaatctgcggtctgcctatagtgagtc

SEQ ID NO: 117 - DNA, 59 bp, Artificial Sequence
  E2F3
ctttgtcccatcgtgcttcagagctgcacccgacttggtcagtctgcctatagtgagtc

SEQ ID NO: 118 - DNA, 63 bp, Artificial Sequence
  LAF4
aaatggctaaacaaagttaatccgccggtaatgctatgctgactcgtctgcctatagtgagtc

SEQ ID NO: 119 - DNA, 63 bp, Artificial Sequence
  LAF4
gagaaagacagctcttcaagactcgtagtgatgcagatgcgctgtgtctgcctatagtgagtc

SEQ ID NO: 120 - DNA, 64 bp, Artificial Sequence
  LAF4
gtcagagagcaatcagtactacaagcccggcataatacagtcctacgtctgcctatagtgagtc

SEQ ID NO: 121 - DNA, 65 bp, Artificial Sequence
  TGFB3
gatggtaagttgagatgttgtgtttgagtcgaagatagccaatcacggtctgcctatagtgagtc

SEQ ID NO: 122 - DNA, 65 bp, Artificial Sequence
  TGFB3
gagatatcctggaaaacattcacgattgggtacaattcggctctagggtctgcctatagtgagtc

SEQ ID NO: 123 - DNA, 64 bp, Artificial Sequence
  TGFB3
gttagaggaaggctgaactctttgttagcatcaggttcgtctaagggtctgcctatagtgagtc

SEQ ID NO: 124 - DNA, 65 bp, Artificial Sequence
  TYMS
gatattgtcagtctttaggggtttgctacagatgatgccgagaagaggtctgcctatagtgagtc

SEQ ID NO: 125 - DNA, 64 bp, Artificial Sequence
  TYMS
gacgacagaagaatcatcatgtcactcctcagattagccgagataagtctgcctatagtgagtc

SEQ ID NO: 126 - DNA, 63 bp, Artificial Sequence
  TYMS
gtcttccaagggagtgaaaattgcgtagaatagctgctcatatcggtctgcctatagtgagtc

SEQ ID NO: 127 - DNA, 65 bp, Artificial Sequence
  WNT10B
acctgaatggactaagatgaaatgaacttatggatttcacgagggcagtctgcctatagtgagtc

SEQ ID NO: 128 - DNA, 64 bp, Artificial Sequence
  WNT10B
gtctccttccattcagatgttatccgaggaccttactttagcagaagtctgcctatagtgagtc

SEQ ID NO: 129 - DNA, 64 bp, Artificial Sequence
  WNT10B
agagtgggtgaatgtgtgtaagcttccgtactgttacaatgtgcgcgtctgcctatagtgagtc

SEQ ID NO: 130 - DNA, 23 bp, Artificial Sequence
  Hsa-miR-103
agcagcattgtacagggctatga

SEQ ID NO: 131 - DNA, 21 bp, Artificial Sequence
  Hsa-miR-339
tccctgtcctccaggagctca

SEQ ID NO: 132 - DNA, 23 bp, Artificial Sequence
  Hsa-miR-183
tatggcactggtagaattcactg

SEQ ID NO: 133 - DNA, 22 bp, Artificial Sequence
  Hsa-miR-182
tttggcaatggtagaactcaca

SEQ ID NO: 134 - DNA, 23 bp, Artificial Sequence
  Hsa-miR-136
agctacattgtctgctgggtttc

SEQ ID NO: 135 - DNA, 23 bp, Artificial Sequence
  Hsa-miR-221
actccatttgttttgatgatgga

SEQ ID NO: 136 - DNA, 59 bp, Artificial Sequence
  Hsa-miR-103
acttcgtcagtaacggactccagtagcgactagcccgtcagcagcattgtacagggcta

SEQ ID NO: 137 - DNA, 58 bp, Artificial Sequence
  Hsa-miR-339
acttcgtcagtaacggactataccggcctaagcactcgcaccctgtcctccaggagct

SEQ ID NO: 138 - DNA, 59 bp, Artificial Sequence
  Hsa-miR-183
acttcgtcagtaacggacaatgttgacccggatctcgtccatggcactggtagaattca

SEQ ID NO: 139 - DNA, 59 bp, Artificial Sequence
  Hsa-miR-182
acttcgtcagtaacggacactagccctcgcatagcttgcgtttggcaatggtagaactc

SEQ ID NO: 140 - DNA, 58 bp, Artificial Sequence
  Hsa-miR-136
acttcgtcagtaacggacgcgcaattccctcgatcttacgctacattgtctgctgggt

SEQ ID NO: 141 - DNA, 60 bp, Artificial Sequence
  Hsa-miR-221
acttcgtcagtaacggacgtaggtcccggacgtaatcaccactccatttgttttgatgat

SEQ ID NO: 142 - DNA, 47 bp, Artificial Sequence
  CTNNA1
tgtccatgcaggcaacataaacttcaagtgggatcctaaaagtctag

SEQ ID NO: 143 - DNA, 42 bp, Artificial Sequence
  CTNNA1
acttcgtcagtaacggacgtgtccatgcaggcaacataaact

SEQ ID NO: 144 - DNA, 42 bp, Artificial Sequence
  CTNNA1
gagtcgaggtcatatcgtgtgtccatgcaggcaacataaact

SEQ ID NO: 145 - DNA, 63 bp, Artificial Sequence
  CTNNA1
caagtgggatcctaaaagtctagcggaaactggcgatcagctagtgtctgcctatagtgagtc

SEQ ID NO: 146 - DNA, 46 bp, Artificial Sequence; identical to SEQ ID NO: 39
  XPO1

SEQ ID NO: 147 - DNA, 41 bp, Artificial Sequence; identical to SEQ ID NO: 9
  XPO1

SEQ ID NO: 148 - DNA, 41 bp, Artificial Sequence; identical to SEQ ID NO: 10
  XP01

SEQ ID NO: 149 - DNA, 63 bp, Artificial Sequence
  XPO1
tactgacacatttaaaggagcatacccacagacgttggtccgtaggtctgcctatagtgagtc

SEQ ID NO: 150 - DNA, 43 bp, Artificial Sequence; identical to SEQ ID NO: 36
  SOX9

SEQ ID NO: 151 - DNA, 39 bp, Artificial Sequence; identical to SEQ ID NO: 3
  SOX9

SEQ ID NO: 152 - DNA, 39 bp, Artificial Sequence; identical to SEQ ID NO: 4
  SOX9

SEQ ID NO: 153 - DNA, 62 bp, Artificial Sequence
  SOX9
ctcacagtacgactacaccgactctgggagtacctagcttcggagtctgcctatagtgagtc

SEQ ID NO: 154 - DNA, 45 bp, Artificial Sequence
  RELA
tccctttacgtcatccctgagcaccatcaactatgatgagtttcc

SEQ ID NO: 155 - DNA, 40 bp, Artificial Sequence
  RELA
acttcgtcagtaacggactccctttacgtcatccctgagc

SEQ ID NO: 156 - DNA, 40 bp, Artificial Sequence
  RELA
gagtcgaggtcatatcgttccctttacgtcatccctgagc

SEQ ID NO: 157 - DNA, 62 bp, Artificial Sequence
  RELA
ccatcaactatgatgagtttccacaggcaagcgtgggtctcatggtctgcctatagtgagtc

SEQ ID NO: 158 - DNA, 45 bp, Artificial Sequence
  PTGDS
agcacctactccgtgtcagtggtggagaccgactacgaccagtac

SEQ ID NO: 159 - DNA, 42 bp, Artificial Sequence
  PTGDS
acttcgtcagtaacggacgagcacctactccgtgtcagtggt

SEQ ID NO: 160 - DNA, 42 bp, Artificial Sequence
  PTGD2
gagtcgaggtcatatcgtgagcacctactccgtgtcagtggt

SEQ ID NO: 161 - DNA, 61 bp, Artificial Sequence
  PTGD2
gagaccgactacgaccagtaccgctgaacgtcaaattgcaggggtctgcctatagtgagtc

SEQ ID NO: 162 - DNA, 44 bp, Artificial Sequence
  EPB49
ccctcagaccaagcacctcatcgaggatctcatcatcgagtcat

SEQ ID NO: 163 - DNA, 40 bp, Artificial Sequence
  EPB49
acttcgtcagtaacggacccctcagaccaagcacctcatc

SEQ ID NO: 164 - DNA, 40 bp, Artificial Sequence
  EPB49
gagtcgaggtcatatcgtccctcagaccaagcacctcatc

SEQ ID NO: 165 - DNA, 61 bp, Artificial Sequence
  EPB49
aggatctcatcatcgagtcatattccaggggagctacgagcgtgtctgcctatagtgagtc

SEQ ID NO: 166 - DNA, 46 bp, Artificial Sequence
  SIM2
tttgtggtagcatctgatggcaaaatcatgtatatatccgagaccg

SEQ ID NO: 167 - DNA, 42 bp, Artificial Sequence
  SIM2
acttcgtcagtaacggacgtttgtggtagcatctgatggcaa

SEQ ID NO: 168 - DNA, 42 bp, Artificial Sequence
  SIM2
gagtcgaggtcatatcgtgtttgtggtagcatctgatggcaa

SEQ ID NO: 169 - DNA, 63 bp, Artificial Sequence
  SIM2
atcatgtatatatccgagaccggcctagtagatcggcgcaatttcgtctgcctatagtgagtc

SEQ ID NO: 170 - DNA, 48 bp, Artificial Sequence
  EDNRA
ggtgtaaaagcagcacaagtgcaataagagatatttcctcaaatttgc

SEQ ID NO: 171 - DNA, 41 bp, Artificial Sequence
  EDNRA
acttcgtcagtaacggacggtgtaaaagcagcacaagtgca

SEQ ID NO: 172 - DNA, 41 bp, Artificial Sequence
  EDNRA
gagtcgaggtcatatcgtggtgtaaaagcagcacaagtgca

SEQ ID NO: 173 - DNA, 65 bp, Artificial Sequence
  EDNRA
taagagatatttcctcaaatttgcggacagtacctacgttggcaaaggtctgcctatagtgagtc

SEQ ID NO: 174 - DNA, 47 bp, Artificial Sequence; identical to SEQ ID NO: 35
  FOXO1A

SEQ ID NO: 175 - DNA, 42 bp, Artificial Sequence; identical to SEQ ID NO: 1
  FOXO1A

SEQ ID NO: 176 - DNA, 42 bp, Artificial Sequence; identical to SEQ ID NO: 2
  FOXO1A

SEQ ID NO: 177 - DNA, 65 bp, Artificial Sequence
  FOXO1A
ggacaacaacagtaaatttgctatcctgtagtaccgggtttgaaagggtctgcctatagtgagtc

SEQ ID NO: 178 - DNA, 40 bp, Artificial Sequence
  FBP1
acttcgtcagtaacggactggcattgctggttctaccaac

SEQ ID NO: 179 - DNA, 40 bp, Artificial Sequence
  FBP1
gagtcgaggtcatatcgttggcattgctggttctaccaac

SEQ ID NO: 180 - DNA, 64 bp, Artificial Sequence
  FBP1
tgacaggtgatcaagttaagaagtcgagcgttcggagcacttaatcgtctgcctatagtgagtc

SEQ ID NO: 181 - DNA, 42 bp, Artificial Sequence
  TNFRSF1A
acttcgtcagtaacggactccccaaggaaaatatatccaccc

SEQ ID NO: 182 - DNA, 42 bp, Artificial Sequence
  TNFRSF1A
gagtcgaggtcatatcgttccccaaggaaaatatatccaccc

SEQ ID NO: 183 - DNA, 63 bp, Artificial Sequence
  TNFRSF1A
caaaataattcgatttgctgtacagtagcccaggtagcggagcttgtctgcctatagtgagtc

SEQ ID NO: 184 - DNA, 42 bp, Artificial Sequence
  NOTCH3
acttcgtcagtaacggacgttcacaggaacctattgcgaggt

SEQ ID NO: 185 - DNA, 42 bp, Artificial Sequence
  NOTCH3
gagtcgaggtcatatcgtgttcacaggaacctattgcgaggt

SEQ ID NO: 186 - DNA, 65 bp, Artificial Sequence
  NOTCH3
gacattgacgagtgtcagagtagctgactcttgtagtattgcgcgaagtctgcctatagtgagtc

SEQ ID NO: 187 - DNA, 40 bp, Artificial Sequence
  ETV1
acttcgtcagtaacggactatgtttgaaaagggccccagg

SEQ ID NO: 188 - DNA, 40 bp, Artificial Sequence
  ETV1
gagtcgaggtcatatcgttatgtttgaaaagggccccagg

SEQ ID NO: 189 - DNA, 65 bp, Artificial Sequence
  ETV1
agttttatgatgacacctgtgtttacggatggcaacaagtacggattgtctgcctatagtgagtc

SEQ ID NO: 190 - DNA, 40 bp, Artificial Sequence
  BID
acttcgtcagtaacggacgttccagcctcagggatgagtg

SEQ ID NO: 191 - DNA, 40 bp, Artificial Sequence
  BID
gagtcgaggtcatatcgtgttccagcctcagggatgagtg

SEQ ID NO: 192 - DNA, 63 bp, Artificial Sequence
  BID
atcacaaacctactggtgtttggcgctaggttaataagcggatgcgtctgcctatagtgagtc

SEQ ID NO: 193 - DNA, 41 bp, Artificial Sequence
  ANXA1
acttcgtcagtaacggacgatcagaattcctcaagcaggcc

SEQ ID NO: 194 - DNA, 41 bp, Artificial Sequence
  ANXA1
gagtcgaggtcatatcgtgatcagaattcctcaagcaggcc

SEQ ID NO: 195 - DNA, 64 bp, Artificial Sequence
  ANXA1
ggtttattgaaaatgaagagcaagggttctatgtttggacgccatggtctgcctatagtgagtc

SEQ ID NO: 196 - DNA, 42 bp, Artificial Sequence
  BCL2
acttcgtcagtaacggaccgtgcctcatgaaataaagatccg

SEQ ID NO: 197 - DNA, 42 bp, Artificial Sequence
  BCL2
gagtcgaggtcatatcgtcgtgcctcatgaaataaagatccg

SEQ ID NO: 198 - DNA, 63 bp, Artificial Sequence
  BCL2
aaggaattggaataaaaatttccggatgacgaccgaataccgttggtctgcctatagtgagtc

SEQ ID NO: 199 - DNA, 42 bp, Artificial Sequence
  CCNG2
acttcgtcagtaacggacgccactcatgatgtgatccggatt

SEQ ID NO: 200 - DNA, 42 bp, Artificial Sequence
  CCNG2
gagtcgaggtcatatcgtgccactcatgatgtgatccggatt

SEQ ID NO: 201 - DNA, 65 bp, Artificial Sequence
  CCNG2
gtcagtgtaaatgtactgcttctggtgctctgagacggcaaagattcgtctgcctatagtgagtc

SEQ ID NO: 202 - DNA, 17 bp, Artificial Sequence
  hsa-miR-647
gtggctgcactcacttc

SEQ ID NO: 203 - DNA, 21 bp, Artificial Sequence
  hsa-miR-647
gtggctgcactcacttccttc

SEQ ID NO: 204 - DNA, 57 bp, Artificial Sequence
  hsa-miR-647
acttcgtcagtaacggacttgagcggacccagatgtaccggtggctgcactcacttc

SEQ ID NO: 205 - DNA, 19 bp, Artificial Sequence
  hsa-miR-519d
agtgcctccctttagagtg

SEQ ID NO: 206 - DNA, 22 bp, Artificial Sequence
  hsa-miR-519d
caaagtgcctccctttagagtg

SEQ ID NO: 207 - DNA, 59 bp, Artificial Sequence
  hsa-miR-519d
acttcgtcagtaacggaccagagtgtccccgtggcgatacagtgcctccctttagagtg

SEQ ID NO: 208 - DNA, 63 bp, Artificial Sequence
  RAD23B
tactacagcagataggtcgagagtagggttcgggttcagacaccggtctgcctatagtgagtc